SEQUENCE LISTING (86 sequences)

SEQ ID NO: 1 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 2 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 3 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 4 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNPNAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 5 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYCLPMSYELIGCLQGDTSKCAREKVCSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 6 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSCNNKYRFAGACMAGVALGVATAATVTAGIALHRSNENAQAIANMKNAIQNTNEAVKQLQLANKQTLAVFDTIRGEINNNIIPVINQLPCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 7 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMCGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILICVGSYLGEGEYSADNVELGPPVVIDKIDIGNQLAGINQTLQNAEDYIEKSEEFLKGINPSI

SEQ ID NO: 8 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTPEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 9 - protein, 483 aa, synthetic construct
MAFLKSAIICYLLFYPHIVKSSLHYDSLSKVGIIKGLTYNYKIKGSPSTKLMVVILIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIATIIGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 10 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTCKCAREKVVSSYVPRFALSDGLVYANCLNTICRCMDTDTPISQSLGTTVSLLDNKKCLVYQVGDILISVGSYLGEGEYSADNVECGPPVVIDKIDIGNQLAGINQTLQNAEDYIEKSEEFLKGINPSI

SEQ ID NO: 11 - protein, 483 aa, synthetic construct
MAFLKSAIICYLLFYPHIVKSSLHYDSLSKVGIIKGLTYNYKIKGSPSTKLMVVILIPNIDGVRNCTQKQFDEYKNLVKNVLEPVKLALNAMLDNVKSGNNKYRFAGAIMAGVALGVATAATVTAGIALHRSNPNAQAIANMKNAIQNTNEAVKQLQLANKQTLAVIATIIGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 12 - protein, 483 aa, synthetic construct
MAFLKSAIICYLLFYPHIVKSSLHYDSLSKVGIIKGLTYNYKIKGSPSTKLMVVILIPNIDGVRNCTQKQFDEYKNLVKNVLEPVKLALNAMLDNVKSGNNKYRFAGAIMAGVALGVATAATVTAGIALHRSNENAQAIANMKNAIQNTPEAVKQLQLANKQTLAVIATIIGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 13 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYCLPMSYELIGCLQGDTCKCAREKVCSSYVPRFALSDGLVYANCLNTICRCMDTDTPISQSLGTTVSLLDNKKCLVYQVGDILISVGSYLGEGEYSADNVECGPPVVIDKIDIGNQLAGINQTLQNAEDYIEKSEEFLKGINPSI

SEQ ID NO: 14 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMPNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 15 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYCTPMTNNMRECLTGSTEKCPRELVCSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 16 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMCGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIICLGKYLGSVNYNSEGIAIGPPVFTDKVDISSQISSMNQSLQQSKDYIKEAQRLLDTVNPSL

SEQ ID NO: 17 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTPEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 18 - protein, 488 aa, synthetic construct
MVVILDKRCYCNLLILILMISECSVGILHYEKLSKIGLVKGVTRKYKIKSNPLTKDIVIIMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLAALIDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 19 - protein, 489 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTECKCPRELVVSSHVPRFALSNGVLFANCISVTCQCQTTGRAISQSGEQTLLMIDNTTCPTAVLGNVIISLGKYLGSVNYNSEGIACGPPVFTDKVDISSQISSMNQSLQQSKDYIKEAQRLLDTVNPSL

SEQ ID NO: 20 - protein, 488 aa, synthetic construct
MVVILDKRCYCNLLILILMISECSVGILHYEKLSKIGLVKGVTRKYKIKSNPLTKDIVIIMIPNVSNMSQCTGSVMENYKTRLNGILTPIKGALEIYKNNTHDLVGDVRLAGVIMAGVAIGIATAAQITAGVALYEAMKNADNINKLKSSIESTPEAVVKLQETAEKTVYVLAALIDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 21 - protein, 488 aa, synthetic construct; identical to SEQ ID NO: 20

SEQ ID NO: 22 - protein, 489 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYCTPMTNNMRECLTGSTECKCPRELVCSSHVPRFALSNGVLFANCISVTCQCQTTGRAISQSGEQTLLMIDNTTCPTAVLGNVIISLGKYLGSVNYNSEGIACGPPVFTDKVDISSQISSMNQSLQQSKDYIKEAQRLLDTVNPSL

SEQ ID NO: 23 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMPNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 24 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYCTPMTASVRECLTGSTDKCPRELVCSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 25 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDCVGDVKLAGVCMAGIAIGIATAAQITAGVALYEAMKNADNINKLKSSIESTNEAVVKLQETAEKTVYVFTALQDYINTNLVPTIDQIPCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 26 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMCGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIICLGKYLGSINYNSESIAVGPPVYTDKVDISSQISSMNQSLQQSKDYIKEAQKILDTVNPSL

SEQ ID NO: 27 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTPEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 28 - protein, 488 aa, synthetic construct
MATQEVRLKCLLCGIIVLVLSLEGLGILHYEKLSKIGLVKGITRKYKIKSNPLTKDIVIIMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLAALIDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 29 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTCKCPRELVVSSHVPRFALSGGVLFANCISVTCQCQTTGRAISQSGEQTLLMIDNTTCTTVVLGNIIISLGKYLGSINYNSESIACGPPVYTDKVDISSQISSMNQSLQQSKDYIKEAQKILDTVNPSL

SEQ ID NO: 30 - protein, 488 aa, synthetic construct
MATQEVRLKCLLCGIIVLVLSLEGLGILHYEKLSKIGLVKGITRKYKIKSNPLTKDIVIIMIPNVSNVSKCTGTVMENYKSRLTGILSPIKGAIELYNNNTHDLVGDVKLAGVVMAGIAIGIATAAQITAGVALYEAMPNADNINKLKSSIESTNEAVVKLQETAEKTVYVLAALIDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 31 - protein, 488 aa, synthetic construct
MATQEVRLKCLLCGIIVLVLSLEGLGILHYEKLSKIGLVKGITRKYKIKSNPLTKDIVIIMIPNVSNVSKCTGTVMENYKSRLTGILSPIKGAIELYNNNTHDLVGDVKLAGVVMAGIAIGIATAAQITAGVALYEAMKNADNINKLKSSIESTPEAVVKLQETAEKTVYVLAALIDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSL

SEQ ID NO: 32 - protein, 488 aa, synthetic construct
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYCTPMTASVRECLTGSTCKCPRELVCSSHVPRFALSGGVLFANCISVTCQCQTTGRAISQSGEQTLLMIDNTTCTTVVLGNIIISLGKYLGSINYNSESIACGPPVYTDKVDISSQISSMNQSLQQSKDYIKEAQKILDTVNPSL

SEQ ID NO: 33 - DNA, 1449 bp, synthetic construct
  source 1..1449: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatc

SEQ ID NO: 34 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 35 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 36 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaaccctaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 37 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattactgcctgcctatgagctacgagctgatcggatgtctgcagggcgacaccagcaagtgcgccagagaaaaggtgtgctccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 38 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtcctgcaacaacaagtacagattcgctggcgcctgcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgttcgacaccatccggggagagatcaacaacaacatcatccccgtgatcaaccagctgccctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 39 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatgtgcggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctgcgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 40 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacacccccgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 41 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcatcctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgccaccatcatcggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 42 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacacctgcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaatgcggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 43 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcatcctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaaccctaacgcccaggccattgccaacatgaagaacgccatccagaacaccaacgaggccgtgaagcagctccagctggccaacaaacagaccctggccgtgatcgccaccatcatcggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 44 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcatcctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacacccccgaggccgtgaagcagctccagctggccaacaaacagaccctggccgtgatcgccaccatcatcggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattacgccctgcctatgagctacgagctgatcggatgtctgcagggcgacaccagcaagtgcgccagagaaaaggtggtgtccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaactgggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 45 - DNA, 1704 bp, synthetic construct
  source 1..1704: mol_type = other DNA
atggcctttctgaagtccgccatcatctgctacctgctgttctaccctcacatcgtgaagtccagcctgcactacgacagcctgagcaaagtgggcatcatcaagggcctgacctacaactacaagatcaagggcagccccagcaccaagctgatggtggtcaagctgatccccaacatcgacggcgtgcggaactgtacccagaagcagttcgacgagtacaagaacctggtcaagaacgtgctggaacccgtgaagctggccctgaacgccatgctggacaatgtgaagtccggcaacaacaagtacagattcgctggcgccatcatggccggcgttgcacttggagttgccacagccgctacagtgacagccggaattgccctgcacagaagcaacgagaacgcccaggccattgccaacatgaagaacgccatccagaacaccaacgaggccgtgaagcagctccagctggccaacaaacagaccctggccgtgatcgacaccatccggggagagatcaacaacaacatcatccccgtgatcaaccagctgagctgtgacaccatcggcctgagcgtgggaatcaagctgacccagtactacagcgagatcctgaccgccttcggacccgctctgcagaaccctgtgaacaccagaatcaccatccaggccatcagcagcgtgttcaaccggaacttcgacgagctgctgaagatcatgggctacaccagcggcgacctgtatgagatcctgcacagcggcctgatccggggcaatatcatcgacgtggacgtggaagccggctatatcgccctggaaatcgagttccccaacctgacactggtgcccaacgccgtggtgcaagagctgatgcccatctcctacaacgtggacggcgacgagtgggttaccctggtgcctagattcgtgctgacccggacaaccctgctgagcaacatcgataccagcagatgcaccgtgaccgagagcagcgtgatctgcgacaacgattactgcctgcctatgagctacgagctgatcggatgtctgcagggcgacacctgcaagtgcgccagagaaaaggtgtgctccagctacgtgcccagattcgccctgtctgacggactggtgtacgccaactgcctgaacaccatctgccggtgcatggacaccgacacacctatctctcagagcctgggcaccaccgtgtctctgctggataacaagaaatgcctggtgtaccaagtgggcgacatcctgatctccgtgggctcttatctcggcgagggcgagtacagcgccgacaatgttgaatgcggccctcctgtggtcatcgacaagatcgacatcggcaatcagctggccggcatcaatcagaccctgcagaacgccgaggactacatcgagaagtctgaagagttcctgaagggcatcaaccccagcatcggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataatgactcgag

SEQ ID NO: 46 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgcctaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 47 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactactgcacccccatgaccaacaacatgagggagtgcctgaccggcagcaccgagaagtgccccagggagctggtgtgcagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 48 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatgtgcggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatctgcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 49 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcacccccgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 50 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcatcatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctggccgccctgatcgactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 51 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcacctgcaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgcctgcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 52 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcatcatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgcctaacgccgacaacatcaacaagctgaagagcagcatcgagagcaccaacgaggccgtggtgaagctgcaggagaccgccgagaagaccgtgtacgtgctggccgccctgatcgactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 53 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcatcatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcacccccgaggccgtggtgaagctgcaggagaccgccgagaagaccgtgtacgtgctggccgccctgatcgactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactacgccacccccatgaccaacaacatgagggagtgcctgaccggcagcaccgagaagtgccccagggagctggtggtgagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgccatcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 54 - DNA, 1464 bp, synthetic construct
  source 1..1464: mol_type = other DNA
atggtggtgatcctggacaagaggtgctactgcaacctgctgatcctgatcctgatgatcagcgagtgcagcgtgggcatcctgcactacgagaagctgagcaagatcggcctggtgaagggcgtgaccaggaagtacaagatcaagagcaaccccctgaccaaggacatcgtgatcaagatgatccccaacgtgagcaacatgagccagtgcaccggcagcgtgatggagaactacaagaccaggctgaacggcatcctgacccccatcaagggcgccctggagatctacaagaacaacacccacgacctggtgggcgacgtgaggctggccggcgtgatcatggccggcgtggccatcggcatcgccaccgccgcccagatcaccgccggcgtggccctgtacgaggccatgaagaacgccgacaacatcaacaagctgaagagcagcatcgagagcaccaacgaggccgtggtgaagctgcaggagaccgccgagaagaccgtgtacgtgctgaccgccctgcaggactacatcaacaccaacctggtgcccaccatcgacaagatcagctgcaagcagaccgagctgagcctggacctggccctgagcaagtacctgagcgacctgctgttcgtgttcggccccaacctgcaggaccccgtgagcaacagcatgaccatccaggccatcagccaggccttcggcggcaactacgagaccctgctgaggaccctgggctacgccaccgaggacttcgacgacctgctggagagcgacagcatcaccggccagatcatctacgtggacctgagcagctactacatcatcgtgagggtgtacttccccatcctgaccgagatccagcaggcctacatccaggagctgctgcccgtgagcttcaacaacgacaacagcgagtggatcagcatcgtgcccaacttcatcctggtgaggaacaccctgatcagcaacatcgagatcggcttctgcctgatcaccaagaggagcgtgatctgcaaccaggactactgcacccccatgaccaacaacatgagggagtgcctgaccggcagcacctgcaagtgccccagggagctggtgtgcagcagccacgtgcccaggttcgccctgagcaacggcgtgctgttcgccaactgcatcagcgtgacctgccagtgccagaccaccggcagggccatcagccagagcggcgagcagaccctgctgatgatcgacaacaccacctgccccaccgccgtgctgggcaacgtgatcatcagcctgggcaagtacctgggcagcgtgaactacaacagcgagggcatcgcctgcggcccccccgtgttcaccgacaaggtggacatcagcagccagatcagcagcatgaaccagagcctgcagcagagcaaggactacatcaaggaggcccagaggctgctggacaccgtgaaccccagcctg

SEQ ID NO: 55 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgcctaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 56 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactactgcacacctatgaccgccagcgtcagagagtgtctgacaggcagcaccgacaagtgccccagagaactggtgtgctccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 57 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgactgcgtgggcgacgtgaaacttgctggcgtttgcatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgttcacagccctgcaggactacatcaacaccaacctggtgcctaccatcgaccagatcccttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 58 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatgtgcggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatctgcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 59 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcacccccgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 60 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcatcatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctggcagccctgatcgactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 61 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcacctgcaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgcctgcggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 62 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcatcatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgcctaacgccgacaacatcaacaagctgaagtccagcatcgagagcaccaacgaggccgtggtcaagctgcaagagacagccgagaaaaccgtgtacgtgctggcagccctgatcgactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 63 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcatcatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcacccccgaggccgtggtcaagctgcaagagacagccgagaaaaccgtgtacgtgctggcagccctgatcgactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactacgccacacctatgaccgccagcgtcagagagtgtctgacaggcagcaccgacaagtgccccagagaactggtggtgtccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgccgtgggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 64 - DNA, 1464 bp, synthetic construct
  source 1..1464: mol_type = other DNA
atggccacacaagaagtgcggctgaagtgcctgctgtgcggcatcattgtgctggtgctgtctctggaaggcctgggcatcctgcactacgagaagctgtctaagatcggcctggtcaagggcatcacccggaagtacaagatcaagagcaaccctctgaccaaggacatcgtgatcaagatgatccccaacgtgtccaatgtgtccaagtgcaccggcaccgtgatggaaaactacaagagccggctgaccggcattctgagccctatcaagggcgccatcgagctgtacaacaacaacacccacgacctcgtgggcgacgtgaaacttgctggcgttgtgatggccggaatcgccatcggaattgccacagccgctcagattacagctggcgtggccctgtatgaggccatgaagaacgccgacaacatcaacaagctgaagtccagcatcgagagcaccaacgaggccgtggtcaagctgcaagagacagccgagaaaaccgtgtacgtgctgacagccctgcaggactacatcaacaccaacctggtgcctaccatcgaccagatctcttgcaagcagaccgagctggccctggatctggccctgagcaagtacctgagcgacctgctgttcgtgttcggccccaatctgcaggaccctgtgtccaacagcatgaccatccaggccatcagccaggcctttggcggcaactatgagacactgctgagaaccctgggctacgccaccgaggacttcgacgatctgctggaaagcgactctatcgccggccagatcgtgtatgtggacctgagcagctactacatcatcgtgcgggtgtacttccccatcctgaccgagatccagcaggcctacgtgcaagagctgctgcccgtgtccttcaacaacgacaacagcgagtggatcagcatcgtgcccaacttcgtgctgatccggaacaccctgatcagcaacatcgaagtgaagtactgcctgatcaccaagaagtccgtcatctgcaaccaggactactgcacacctatgaccgccagcgtcagagagtgtctgacaggcagcacctgcaagtgccccagagaactggtgtgctccagccacgtgccaagatttgctctgtctggcggcgtgctgttcgccaactgtatcagcgtgacctgccagtgccagaccacaggcagagccatttctcagagcggcgaacagaccctgctgatgatcgacaacaccacctgtaccaccgtggtgctgggcaacatcatcatcagcctgggcaagtatctgggcagcatcaactacaactccgagagcattgcctgcggacctccagtgtacaccgataaggtggacatcagcagccagatcagcagcatgaaccagagcctgcagcagagcaaggattacatcaaagaggcccagaaaatcctggacaccgtgaatcccagcctc

SEQ ID NO: 65 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSPENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 66 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRPNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 67 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHPSNENAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 68 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNEPAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 69 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENPQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 70 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAPAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 71 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSPPNAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 72 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSPEPAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 73 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNPPAQAIANMKNAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 74 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKPAIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 75 - protein, 483 aa, synthetic construct
MAFLKSAIICYLLFYPHIVKSSLHYDSLSKVGIIKGLTYNYKIKGSPSTKLMVVKLIPNIDGVRNCTQKQFDEYKNLVKNVLEPVKLALNAMLDNVKSGNNKYRFAGAIMAGVALGVATAATVTAGIALHRSNENAQAIANMKPPIQNTNEAVKQLQLANKQTLAVIDTIRGEINNNIIPVINQLSCDTIGLSVGIKLTQYYSEILTAFGPALQNPVNTRITIQAISSVFNRNFDELLKIMGYTSGDLYEILHSGLIRGNIIDVDVEAGYIALEIEFPNLTLVPNAVVQELMPISYNVDGDEWVTLVPRFVLTRTTLLSNIDTSRCTVTESSVICDNDYALPMSYELIGCLQGDTSKCAREKVVSSYVPRFALSDGLVYANCLNTICRCMDTDTPISQSLGTTVSLLDNKKCLVYQVGDILISVGSYLGEGEYSADNVELGPPVVIDKIDIGNQLAGINQTLQNAEDYIEKSEEFLKGINPSI

SEQ ID NO: 76 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAPKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 77 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEPMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 78 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYPAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 79 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKPADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 80 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNPDNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 81 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNAPNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 82 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAPPNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 83 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAPKPADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 84 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMPPADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 85 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKPSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

SEQ ID NO: 86 - protein, 488 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKPPIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSL

(19) United States
(12) Patent Application Publication
(10) Pub. No.: US 2024/0358817 A1
(43) Pub. Date: Oct. 31, 2024

(54) STRUCTURES OF LANGYA VIRUS FUSION PROTEIN ECTODOMAIN AND IMMUNOGENIC COMPOSITIONS DERIVED THEREFROM

(71) Applicant: Duke University, Durham, NC (US)

(72) Inventors: Priyamvada Acharya, Durham, NC (US); Aaron May, Durham, NC (US)

(21) Appl. No.: 18/631,993

(22) Filed: Apr. 10, 2024

Related U.S. Application Data
(60) Provisional application No. 63/458,400, filed on Apr. 10, 2023.

(51) Int. Cl. A61K 39/155; A61P 31/14; C07K 14/005; C12N 7/00; G01N 21/64; G01N 33/569; G01N 33/68

(52) U.S. Cl. CPC A61K 39/155; A61P 31/14; C07K 14/005; C12N 7/00; G01N 21/6486; G01N 33/56983; G01N 33/68; C12N 2760/18222; C12N 2760/18234; G01N 2333/115

(57) ABSTRACT

Described are mutations to the fusion protein and related domains of Henipaviruses and their use in creating immunogens useful for vaccine compositions, and in prevention and treatment of Paramyxovirus and Henipavirus infections in a mammal. Also described are methods for screening viral fusion proteins for capability to transition from a pre-fusion to a post-fusion conformation.

### CROSS-REFERENCE TO RELATED APPLICATIONS

This application claims the benefit of and priority to U.S. Provisional Application No. 63/458,400, filed on Apr. 10, 2023, the entire contents of which are incorporated herein by reference.

### STATEMENT OF GOVERNMENTAL INTEREST

This invention was made with government support under grant nos. NIAID R01 AI145687, R01 AI165947, R01 AI165147 and U54 AI170752. The government has certain rights in the invention.

### SEQUENCE LISTING

This application herein contains a Sequence Listing XML. It has been submitted electronically via the USPTO Patent Center as an XML file entitled [DU8099US . . . ] Sais XML file, created on May 15, 2024 is 20,217 bytes in size. It is hereby incorporated by reference in its entirety.

### BACKGROUND OF THE INVENTION

Langya virus (LayV) is a newly identified member of the Henipavirus genus, detected after surveillance of disease following animal exposure in eastern China (21). Over the course of several years, LayV infections were identified in 35 individuals, 26 of whom were infected solely with LayV. In these 26 individuals, common symptoms included fever, fatigue, cough, nausea, and headaches. The genome organization of LayV is identical to that of other Henipaviruses, including the better known and highly virulent Nipah (NiV) and Hendra (HeV) viruses. Based on phylogenetic analysis, LayV is most closely related to Mojiang virus (MojV), which was discovered in rats in southern China (22), and Gamak (GAKV) and Daeryong (DARV) viruses, which were detected in shrews in the Republic of Korea (23).

The Henipaviruses infect a range of animals with Nipah and Hendra viruses having their natural reservoir in fruit bats (24). However, several additional Henipavirus species have been discovered in recent years with differing animal reservoirs. A survey of both domestic and wild animals revealed a low level of seropositivity for LayVin goats and dogs (2% and 5% respectively) and the highest positivity rate (52.1%) in shrews (Crocidura lasiura specifically), indicating that shrews may be the natural reservoir for LayV. The Henipavirus genus is a member of the Paramyxoviridae family (25), which includes both extremely infectious human pathogens, such as measles and mumps, and extremely deadly pathogens, such as Nipah and Hendra. This diversity of infectivity and virulence, as well as diversity of animal reservoirs are risk factors that necessitate Paramyxovirus research. Accordingly, Nipah and Hendra virus are listed as priority pathogens by the WHO (26).

Henipaviruses present two surface glycoproteins known as the attachment and fusion proteins. These proteins work together to mediate viral entry into host cells and in Nipah virus both have been shown to be required for viral entry (27). As these are the sole virus surface-expressed proteins, they are also the primary targets of neutralizing antibodies against henipaviruses. The Henipavirus attachment protein has receptor-binding capability and, in Nipah and Hendra viruses, binds to Ephrin B2 and B3, which is found mostly in the brain and in endothelial cells in the heart and lungs (28, 29).

The Henipavirus fusion (F) protein is a class-I fusion protein that has a metastable pre-fusion conformation which is displayed on virion surfaces prior to receptor engagement and a post-fusion conformation that is adopted after virus-cell fusion (30, 31). Proteolytic cleavage splits F into two subunits, F1 and F2, still connected by disulfide linkages (30, 32), freeing a series of hydrophobic residues known as the fusion peptide to be inserted into the host cell membrane during this conformational conversion. This process anchors the virus to the host cell and allows the formation of a six-helix bundle in the F protein to bring the viral and host membranes together, facilitating fusion. Although it has many mechanistic features in common with other class-I fusion proteins, the F protein in paramyxoviruses does not appear to have any primary receptor-binding functionality. This role is fulfilled by a separate attachment glycoprotein, which is believed to undergo conformational changes during receptor binding which pass a triggering signal to F (33, 34). The structural basis for this process is poorly understood.

### SUMMARY OF THE INVENTION

The continuing emergence of new paramyxovirus species necessitates heightened focus on a family that includes both some of the world's deadliest pathogens in Nipah virus, and most infectious in measles and mumps. Members specifically of the Henipavirus genus have spread to humans through zoonotic transfer. While Nipah and Hendra viruses are spread to humans through bats, new Henipaviruses have been detected in a range of species, such as shrews and rats. The role of animal reservoirs in the SARS-COV-2 pandemic highlights the risk factor that a diverse range of animal hosts can be responsible, further necessitating efforts to develop treatments or vaccines against Henipaviruses. Structural determinations of Henipavirus glycoproteins serve as a foundation for future immunogen or therapeutic design. Herein, these structural determinations in the LayV fusion protein have provided insight into conformation changes in the protein and have led to the design of mutations that can stabilize the pre-fusion conformation of the protein. In some embodiments, LayV fusion proteins containing one or more of these mutations can be superior immunogens for prophylactic or therapeutic vaccines.

This invention describes stabilizing mutations that were designed based on atomic level structures of the fusion protein of Langya virus, a recently identified member of the Henipavirus genus. These mutations were designed based on a novel observation made on the newly resolved Langya virus fusion protein structure that the fusion peptide was contained within an interprotomer pocket and stabilization of this interaction would stabilize the pre-fusion conformation of the F protein. Additionally, a kink was observed in the HRA helix that can be a source of metastability. Stabilizing this kink can stabilize the pre-fusion conformation by preventing the conformational change to the post-fusion conformation. Further, because the architecture of the pre-fusion F protein is highly conserved across the Henipavirus genus it is expected that the stabilization strategies described would be applicable to all members of the Henipavirus genus, and potentially also extend to the larger Paramyxovirus family.

The stabilizing mutations are based on determination of cryo-EM structures of the Lay V fusion protein (LayV-F) in pre- and post-fusion conformations, both at 4.64 Å resolution. The LayV-F ectodomain sequence as reported by Zhang et al. (2022) was used (21), replacing the transmembrane and cytoplasmic domains with purification tags and a trimerization domain. We compared it to other known Henipavirus fusion protein structures and elucidated the structural basis for pre- to post-fusion conversion in both LayV and more broadly for Henipavirus fusion proteins.

The present study demonstrated that the highly conserved paramyxovirus fusion protein architecture is utilized by LayV-F, identifies a region of variability among Henipavirus fusion proteins in an important antigenic site, and provides evidence for a mechanism to describe how Henipavirus fusion proteins are triggered to undergo conformational changes during the fusion process.

Investigating these structures revealed potential mechanisms of pre-fusion to post-fusion structural conversion based on which we have designed mutations that can stabilize the pre-fusion conformation of LayV-F. Such stabilized pre-fusion F protein ectodomains can have utility as immunogens for vaccine development against Paramyxoviruses/Henipaviruses, which include pathogens such as Nipah and Hendra. While these designs are based on the Langya virus F protein structure, these can be translatable to the entire family of viruses.

Thus, we now have identified mutational targets to this fusion protein domain which result in potential antigenic targets for broad spectrum immunogenic compositions in both Henipavirus and Paramyxovirus species.

In accordance with a first embodiment, the present invention provides a method for generating a mutant Henipavirus fusion protein antigen wherein the method comprises identifying specific amino acids which stabilize a pre-fusion conformation of the Henipavirus fusion protein.

In accordance with a second embodiment, the present invention provides a Langya Henipavirus fusion protein antigen having an amino acid sequence at least 80% identical to SEQ ID NOs. 1-3 and wherein the antigen sequence comprises one or more of the following mutations:
a) stabilizing a kinked conformation by mutation of Glutamic Acid 134 to Proline;
b) stabilizing the DII domain by mutation of alanine 340 to cysteine and/or valine 364 to cysteine;
c) making one or more of the following mutations: glycine 99 to cysteine, isoleucine 109 to cysteine, isoleucine 167 to phenylalanine and serine 186 to proline;
d) stabilizing the DII domain by mutation of alanine 111 to cysteine and serine 423 to cysteine;
e) inhibition of HRA helix region to straighten its conformation by mutation of asparagine 150 to proline;
f) stabilization of prefusion HRA domain conformation by mutation of charged amino acids to non-charged amino acids lysine 55 to isoleucine, aspartic acid 168 to alanine, and arginine 171 to isoleucine;
g) stabilizing the HRB domain by mutation of serine 356 to cysteine and lysine 440 to cysteine;
h) a combination of the mutations of a) and f); and
i) the combination of mutations of b) and g).

In accordance with a third embodiment, the present invention provides an immunogenic composition comprising any of the Henipavirus fusion protein antigens, and a pharmaceutically acceptable carrier.

In a fourth embodiment, the invention provides methods for generating an immune response in a subject, comprising administering to the subject, an effective amount of the immunogenic composition containing any of the Henipavirus fusion protein antigens.

In a fifth embodiment, the invention provides methods for treatment of, of immunization against, a Paramyxovirus and/or Henipavirus infection in a subject, comprising administering to the subject, an effective amount of the immunogenic composition containing any of the Henipavirus fusion protein antigens.

In a sixth embodiment, the invention provides for use of an effective amount of any of the immunogenic compositions disclosed herein to treat or immunize against a Paramyxovirus and/or Henipavirus infection in a subject.

In a seventh embodiment, the invention provides for nucleotide sequences encoding the Henipavirus fusion protein antigens.

In an eighth embodiment, the invention provides for vectors encoding the nucleotide sequences encoding the Henipavirus fusion protein antigens.

In a ninth embodiment, the invention provides for a cell comprising the nucleotide sequences encoding the Henipavirus fusion protein antigens and/or the vectors containing the nucleotide sequences.

In a tenth embodiment, Henipavirus fusion proteins at least 80% identical to SEQ ID NOs: 65-86 are disclosed.

In an eleventh embodiment, a method for screening a viral fusion protein for capability to transition from a pre-fusion to a post-fusion conformation that uses differential scanning fluorimetry (DSF) is disclosed.

In a twelfth embodiment, a method for screening a viral fusion protein for capability to transition from a pre-fusion to a post-fusion conformation that uses differential binding of antibodies to the viral fusion protein dependent on pre-fusion or post-fusion conformation of the viral fusion protein.

Other embodiments are disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: Structure of LayV-F in pre- and post-fusion conformation. Cryo-EM maps of the pre- and post-fusion conformations of LayV-F along with the fitted models with a sequence key. 1A-1B. Cryo-EM reconstructions of LayV-F in 1A. pre- and 1B. post-fusion conformation, colored by protomer. Map regions colored in different shades of blue (pre-fusion) and yellow (post-fusion) for each protomer. Representative reference-free 2D classes used for the reconstruction are displayed above each map. 1C-1D. Cartoon representation of atomic models of LayV-F in 1C. pre-fusion and 1D. post-fusion conformation that were built into the maps shown in 1A-1B. In each, one protomer is colored by domain, based on the color scheme in panel 1E, and the other two protomers colored gray. 1E. Sequence domain key for LayV-F. SS: Secretion signal, cleaved prior to purification. DI, DII, DIII: Domains I, II, and III. FP: Fusion Peptide. HRA, HRB: Heptad repeats A and B. Foldon: Trimerization domain. TS: Twin-Strep tag, used for purification with modified streptavidin resin.

FIGS. 2A-2D: Role of conserved cysteines during conformational conversion. Demonstration of the role of conserved cysteines in each domain in pre- to post-fusion conformational changes. 2A. Zoomed-in view of the DIII domain, including the fusion peptide (orange) and HRA (yellow), shown for the (left) pre- and (right) post-fusion structures. Color scheme continued from FIG. 1E. The cleavage site at R104 is identified, as well as the disulfide bond formed by C66 and C187 (yellow sticks). This disulfide bond is a hinge point around which the HRA straightens into the central coiled-coil in the post-fusion F structure. 2B-2C. Superimposition of pre- and post-fusion conformation domains DI (2B) and DII (2C). Color scheme continued from FIG. 1E, with the post-fusion conformation colored in a lighter shade. Disulfide bonds are shown as yellow sticks and labelled. For DII, the movement of the HRB linker region is indicated by an arrow. 2D. Sequence alignment of paramyxovirus fusion proteins in the regions surrounding conserved cysteines, with cysteine residue number and domain labelled. Viruses colored blue are from the Henipavirus genus and colored tan for other paramyxoviruses. Residues are colored by type, per MView standard (1). Blue: Alcohol, Green: Hydrophobic, Dark Blue: Negative charge, Red: Positive charge, Purple: Polar, Yellow: Cysteine.

FIGS. 3A-3F: Fusion peptide burial and conservation. Exploration of motion in the pre-F structure through B-factor and DDM analysis with focus on the features and sequence conservation of the fusion peptide pocket. 3A. Structure of the pre-fusion LayV-F protein shown in cartoon representation and colored by B-factors. 3B. Difference Distance Matrix plot comparing relative residue positions between pre-fusion and post-fusion conformation. Sequence key from FIG. 1E included along each axis. Gray areas in the bottom right half of the plot are the result of sequences not present in the post-fusion model. 3C. Hydrophobic fusion peptide pocket in pre-fusion state, with fusion peptide depicted as orange cartoon, and surface electrostatics view of the pocket. 3D. Side-chain stick view of the fusion peptide pocket, with hydrophobic residues involved shown with side chains as green sticks. 3E. Cartoon representations of a single pre-fusion state protomer, colored by sequence conservation across Henipavirus fusion proteins. Color scale generated by ChimeraX default AL2CO entropy measure. 3F. Zoom in of the fusion peptide pocket, colored by sequence conservation as in H. The fusion peptide pocket is among the most strongly conserved region of the protein.

FIGS. 4A-4F: Structure and antigenicity comparison of Henipavirus fusion proteins. Comparison of LayV-F structure, surface residue types, and glycosylation pattern to previously determined NiV-F and HeV—F structures, with focus on important antigenic sites. 4A. Superimposition of a single pre-fusion protomer of LayV-F (blue) NiV-F (cyan) and HeV-F (green), in cartoon representation. PDBs: LayV-F: 8FEJ, NiV-F: 5EVM, HeV-F: 5EJB (2, 3). 4B. Zoomed in view of a loop within the folded HRA, one of the few areas of significant structural difference between LayV-F and NiV/HeV-F. 4C. Binding site of known paramyxovirus F protein-neutralizing antibodies, labeled by the species targeted. NiV-F trimer from 12B2-bound structure shown as cartoon and antibody Fabs shown in surface view. Only one Fab per type shown, all expect PIA174 bind with 3:1 stoichiometry. PDBs: PIA174: 6MJZ, 12B2: 7KI4, mAb66: 6T3F, 1F5: 7KI6, 5B3: 6TYS (4-6). 4D. Surface view of Lay V-F and NiV-F with glycosylation sites (residues meeting the N-X-S/T glycosylation sequence) colored teal. 4E. Surface representation of LayV-F colored by sequence conservation across Henipavirus fusion proteins. Color scale generated by ChimeraX default AL2CO entropy measure; magenta indicates high conservation and teal indicates high variability (7). 4F. Surface representation of paramyxovirus fusion proteins, showing only main chain atoms, colored by residue type and shown from top-down view. Tan: hydrophobic, Pink: polar, Red: negative, Blue: positive, teal: Potential glycosylation. PIV5 PBD: 4GIP (8)

FIGS. 5A-5B: Fusion triggering hypothesis for paramyxoviruses. Proposed mechanism of Henipavirus fusion triggering based on new data and previous knowledge. 5A. Existing model of Henipavirus (HNV) fusion protein triggering based on Liu et al. (2013) (9). HNV-G, brown, shown in a two-receptor binding domain down “closed” state, bound to HNV-F in pre-fusion conformation. Exposure to Ephrin B2/B3 receptor induces opening of HNV-G, triggering conversion of HNV-F from pre-fusion to the pre-hairpin state. The triggering interaction is labeled with a star. 5B. Hypothesized structural basis for the G-F interaction shown in A, wherein a two-step mechanism involving disruption of the fusion peptide burial pocket through action of the HNV-G stalk on DII frees the fusion peptide, allowing conformational conversion.

FIG. 6: Sequence Alignment of Paramyxoviridae Fusion Proteins. Color-coded sequence alignment of select Paramyxoviridae Fusion proteins compared to the Lay V-F protein. Sequence alignment of select Paramyxovirus fusion proteins with LayV-F used as the reference sequence. Cov: percent sequence coverage. Pid: percent sequence identity. Residues are colored by type, per MView standard (1). Blue: Alcohol, Green: Hydrophobic, Dark Blue: Negative charge, Red: Positive charge, Purple: Polar, Yellow: Cysteine. Consensus residue types assigned per MView standard. Alcohol: (o), Aliphatic: (l), Aromatic: (a), Charged: (c), Hydrophobic: (h), Negative: (−), Polar: (p), Positive: (+), Small: (s), Tiny: (u), Turnlike: (t).

FIG. 7A-7F: Purification and Antibody Binding of Henipavirus Proteins. Purification and quality control results for several Henipavirus fusion protein constructs with BLI binding data of anti-NiV antibodies against these constructs. 7A. Size-exclusion chromatogram of Henipavirus fusion protein purification. LayV-F, blue, NiV-F WT, dashed red, pre-fusion stabilized NiVop08 (10), dashed green. The fractions identified as the ectodomain trimer at 180 kDa are indicated with a vertical dashed line. 7B. Differential scanning fluorimetry profiles of the products from 7A, LayV-F measured in triplicate. 7C-7E. SDS-PAGE of Henipavirus constructs from 7A. Molecular weight markers, MW, non-reducing conditions, NR, reducing conditions, R., 7C. LayV-F loaded at 1 μg per lane. 7D. LayV-F loaded at 8 μg per lane. 7E. NiVop08 loaded at 3 μg per lane, with left two bands sampling the first peak at 440 kDa and the second two bands sampling the trimer peak at 180 kDa. 7F. BLI sensorgram of anti-NiV-F antibodies h5B3.1 and 12B2 binding to immobilized LayV-F or NiVop08 (6, 11).

FIG. 8: Cryo-EM processing of Lay V-F structures. Cryo-EM processing of Lay V-F in pre- and post-fusion conformation, showing representative micrographs, 2D classes, intermediate 3D refinements, and workflow. Cryo-EM processing of pre- and post-fusion LayV-F structures. Steps performed using CryoSPARC (12, 13). CTF Estimation performed on micrographs followed by default blob picker algorithm and a trained AI-based algorithm (Topaz) for particle picking. Particles for the post-fusion state were obtained through the blob picker, while particles contributing to the pre-fusion state came from both sets. Heterogeneous refinement was used to classify and further sort particles. After non-uniform refinement, a local refinement was performed on the entire structure. The supplemental map of the post-fusion state prior to local refinement where the N65 glycan is detected has also been deposited.

FIG. 9: Fusion Peptide Pocket. Enlarged view of a panel from FIG. 3 depicting the fusion peptide burial pocket. Enlarged view of FIG. 3D. Side-chain stick view of the fusion peptide pocket, with hydrophobic residues involved labelled and shown with side chains as green sticks.

FIG. 10: Illustrates a view of the E134P mutation. A kinked conformation is introduced to a helix by addition of a proline. The mechanism of fusion activation for LayV-F predicts that the kinked helix that comprises a portion of the fusion peptide and the beginning of HRA can be a high-energy state, held in place by the fusion peptide pocket. Disruption of this pocket allows for straightening of this helix, relieving the tension. By placing a proline near the kink point, it can stabilize this conformation. Key: FP, Orange; HRA, Yellow; E134P site, Red.

FIG. 11: Illustrates a view of the A340C and V364C mutations. This is an interprotomer interaction between two DI regions. This can help to lock the DII domain in place, preventing activation. Key: Green, DI (for both protomers involved); Red, DII; Orange, FP; Purple, Mutations.

FIG. 12: Illustrates a view of the G99C, I109C, I167F and S186P mutations. This set of mutations is similar to the NiVop08 mutations (doi.org/10.3389/fi mmu.2020.00842). Key: Chains, Shades of blue; Mutations, Red.

FIG. 13: Illustrates a view of the A111C and S423C mutations. Based on a mechanism by which DII could be moved, freeing the FP, this mutation can lock DII in place, attaching the upper end to a part of the FP. Key: FP, Orange; DII, Red; Mutations, Purple.

FIG. 14: Illustrates a view of the N150P mutation. This linker connects the first part of the HRA helix to the HRA loop in the pre-F conformation. This entire region can straighten into a helix, but a proline at this position can prevent that. Key: Yellow, HRA; Protomers, Shades of Blue; Red, Mutation.

FIG. 15: Illustrates a view of the K55I, D168A and R171I mutations. These mutations affect three charged residues that are buried within the HRA pre-F fold. The early conformational change in the conversion process can disrupt the interaction of these residues such that the buried charged residues become a source of instability, driving further conformational changes. The mutations to hydrophobic residues can stabilize the pre-F conformation. The larger K and R have been changed to I, while the smaller D has been changed to A. Key: Tan, Hydrophobic; Pink, Polar; Blue, Positive; Red, Negative; Cyan, Mutations.

FIG. 16: Illustrates a view of the S356C and L440C mutations. The HRB region loops around to the apex to form the post-F state. With a disulfide linkage here, the linker region can be locked in place, preventing this conversion. Key: Protomers, Blue; HRB, Magenta; Mutations, Red.

FIG. 17: Illustrates example Negative Stain Electron Microscopy (NSEM) of NVAM01 (Nipah virus) and LVAM01 (Langya virus) F proteins.

FIG. 18: Illustrates an example differential scanning fluorimetry (DSF) profile for several proteins.

FIG. 19: Illustrates an example differential scanning fluorimetry (DSF) profile for several proteins.

FIG. 20: Illustrates electron micrographs of various proteins from the experiments illustrated in FIG. 19.

FIG. 21: Illustrates example bio-layer interferometry (BLI) results of antibody binding to various conformations of NiV F protein.

FIG. 22: Illustrates example bio-layer interferometry (BLI) results of antibody binding to various conformations of NiV F protein.

FIG. 23: Illustrates example data from mouse immunizations with Langya virus F proteins.

### DETAILED DESCRIPTION OF THE INVENTION

The biological function of the Henipavirus fusion protein is to transition from pre-fusion to post-fusion. This conversion allows viruses to enter cells. It is generally the case that antibodies are most effective, and perhaps only effective, if they are able to target and neutralize the pre-fusion form, hence the focus on pre-fusion stabilization. In some embodiments, mutations within Henipavirus fusion proteins are disclosed that can lock the protein in its pre-fusion state of conformation.

There can be a difference between forcing the fusion protein to stay in the pre-fusion form in a manner that prevents it from going through its normal biological process (locking) and making the process more energetically unfavorable to occur, while still being possible (stabilization). In some embodiments, disclosed herein are mutations with Henipavirus fusion proteins that can stabilize the proteins in the pre-fusion state or conformation without fully locking.

In some embodiments, disclosed herein are Henipavirus fusion proteins that are stabilized in a post-fusion state or conformation. In some embodiments, these post-fusion stabilized proteins can have utility as reagents for binding or serologic assays. For example, immunization with a stabilized post-fusion form can be used in assays to detect how much of the antibody response in an individual is generated against the post-fusion form of a Henipavirus fusion protein.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

### Definitions

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

The term “administering” can refer to introducing a substance into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. For example, “parenteral administration” can refer to administration via injection or infusion. Parenteral administration includes, but is not limited to, subcutaneous administration, intravenous administration, and intramuscular administration. For example, the inhibitor and/or degrader can be administered intranasally, by inhalation, intrapulmonarily, or by injection (e.g., intravenous or subcutaneous). Herein, administering can refer to introducing an epitope scaffold or composition thereof into a subject. In some embodiments, the purpose of the administration is prophylactic protection against coronavirus infection and/or symptoms of disease caused by coronavirus infection.

The term “simultaneous administration” can refer to a first agent and a second agent administered less than about 15 minutes apart, e.g., less than about 10, 5, or 1 minutes. When the first agent and the second agent are administered simultaneously, the first and second treatments can be in the same composition (e.g., a composition comprising both the first and second therapeutic agents) or separately (e.g., the first therapeutic agent is contained in one composition and the second treatment is contained in another composition).

The term “sequential administration” can refer to a first agent and a second agent administered to a subject greater than about 15 minutes apart, such as greater than about 20, 30, 40, 50, 60 minutes, or greater than 60 minutes apart. Either agent can be administered first. For example, the first agent and the second agent can be included in separate compositions, which can be included in the same or different packages or kits.

The terms “co-administration” or the like, as used herein, can refer to the administration of a first active agent and at least one additional active agent to a single subject, and is intended to include treatment regimens in which the compounds and/or agents are administered by the same or different route of administration, in the same or a different dosage form, and at the same or different time.

As used herein, the term “domain” can refer to a functional portion, segment or region of a protein or polypeptide. “Interaction domain” can refer to a portion, segment or region of a protein, polypeptide or protein fragment that is responsible for the physical affinity of that protein, protein fragment or isolated domain for another protein, protein fragment or isolated domain.

The term “in combination” can refer to the use of more than one therapies (e.g., one or more prophylactic and/or therapeutic agents). The use of the term “in combination” does not restrict the order in which therapies are administered to a subject with a disease or disorder, or the route of administration.

The term “epitope” can refer to a protein determinant capable of specific binding to an immunoglobulin, a scFv, a T-cell receptor and the like.

The term “grafting” can refer to combining an epitope with a protein scaffold. Generally, as described herein, computational methods can be used to identify protein scaffolds that have regions that have the same shape or conformation as a selected epitope (e.g., the scaffolds have regions that can display the epitope such that an immune response to a desired conformation of the epitope can be produced). “Grafting” of the epitope into the protein scaffold at this region (e.g., the epitope can replace the same shape/conformation segment of the protein scaffold) produces the epitope scaffold. “Transplanting” can used instead of grafting.

As used herein, the term “immunogen” and related terms “immunogenic” refer to molecules that have the ability to induce an immune response, including antibodies and/or cellular immune responses in an animal, e.g., a mammal. Although an immunogen may be antigenic, an “antigen” need not necessarily be an “immunogen” because such molecules may not induce a sufficient immune response. In some examples, this may be because of the antigen's size, conformation and the like. In some embodiments, an immunogenic composition can contain one or more immunogens that can induce an immune response that can specifically recognize viruses that contain the immunogen or antigen.

As used herein, the terms “immunological binding,” and “immunological binding properties” can refer to non-covalent interactions of the type which occur between an immunoglobulin molecule and an antigen for which the immunoglobulin is specific. The strength, or affinity of immunological binding interactions can be expressed in terms of the equilibrium binding constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables the cancellation of all parameters not related to affinity, and is equal to the equilibrium binding constant, K_{D}.

The terms “prevent,” “preventing” and/or “prevention” can refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. For example, one or more of the following effects can result from the administration of a therapy or a combination of therapies as described herein: (i) the inhibition of the development or onset of a viral infection and/or a symptom associated therewith; and (ii) the inhibition of the recurrence of a viral infection and/or a symptom associated therewith.

The term “in vivo” can refer to an event that takes place in a subject's body.

The term “in vitro” can refer to an event that takes places outside of a subject's body.

The term “ex vivo” can refer to outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof. In this context, the present compounds can be in numerous applications, both therapeutic and experimental.

The terms “manage,” “managing,” and “management,” in the context of the administration of a therapy to a subject, can refer to the beneficial effects that a subject derives from a therapy, which does not result in a cure of a viral infection. In embodiments, a subject is administered one or more therapies to manage a viral infection so as to prevent the progression or worsening of the viral infection.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intratracheal, intramuscular, subcutaneous, by stent-eluting devices, catheters-eluting devices, intravascular balloons, inhalational and the like. In some embodiments, an immunogenic composition is a type of pharmaceutical composition.

As used throughout the present application, the term “polypeptide” is used in its broadest sense to refer to a sequence of subunit amino acids. The polypeptides described herein may be chemically synthesized or recombinantly expressed. Polypeptide can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It can be generated in any manner, including by chemical synthesis.

Peptides, polypeptides and proteins can be said to have amino acid “substitutions.” Such substitutions refer to replacement of an amino acid at a specific position in a peptide, polypeptide or protein with a different amino acid. In some embodiments, an amino acid substation is said to be “conservative.” A “conservative amino acid substitution” can be a substitution in which an amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In some embodiments, families of amino acid side chains can be grouped, as known in the art, using other properties or characteristics.

“Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

The term “subject,” “patient” or “individual” can refer to any organism to which aspects of the invention can be administered, e.g., for experimental, diagnostic, prophylactic, and/or therapeutic purposes. For example, subjects to which compounds of the disclosure can be administered include animals, such as mammals. Non-limiting examples of mammals include primates, such as humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

The terms “therapies” and/or “therapy” can refer to any protocol(s), method(s), compositions, formulations, and/or agent(s) that can be used in the prevention, treatment, management, or amelioration of a viral infection or a symptom associated therewith. In embodiments, the terms “therapies” and “therapy” can refer to biological therapy, supportive therapy, and/or other therapies useful in treatment, management, prevention, or amelioration of a viral infection or a symptom associated therewith known to one of skill in the art.

The terms “therapeutic agent”, and “therapeutic agents” can refer to any agent(s) which can be used in the prevention, treatment and/or management of a viral infection or a symptom associated therewith.

The term “therapeutically effective amount” can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing.

The terms “treat,” “treatment,” and “treating” can refer to the management and care of a subject for the purpose of combating a condition, disease or disorder, such as a viral infection, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications.

A “variant” can refer to a virus having one or more mutations as compared to a known virus. A strain can be a genetic variant or subtype of a virus. The terms “strain”, “variant,”, and “isolate” may be used interchangeably. In certain embodiments, a variant has developed a “specific group of mutations” that causes the variant to behave differently than that of the strain it originated from.

The term “viral infection” can refer to the invasion by, multiplication and/or presence of a virus in a cell or a subject.

In one embodiment, a viral infection can be an “active” infection. An active infection can refer to one in which the virus is replicating in a cell or a subject. Active infections can be characterized by the spread of the virus to other cells, tissues, and/or organs, from the cells, tissues, and/or organs initially infected by the virus.

In embodiments, the viral infection can be a “latent” infection. A latent infection can refer to one in which the virus is not replicating. In some embodiments, an infection can refer to the pathological state resulting from the presence of the virus in a cell or a subject, or by the invasion of a cell or subject by the virus.

The term “protein scaffold” or “scaffold polypeptide” can refer to a molecule that can be a “framework” for or that can “host” an epitope. A protein scaffold containing a “grafted epitope” is called an epitope scaffold or ES.

The term “grafting” can refer to combining an epitope with a protein scaffold. Generally, as described herein, computational methods can be used to identify protein scaffolds that have regions that have the same shape or conformation as a selected epitope (e.g., the scaffolds have regions that can display the epitope such that an immune response to the epitope can be produced). “Grafting” of the epitope into the protein scaffold at this region (e.g., the epitope can replace the same shape/conformation segment of the protein scaffold) produces the epitope scaffold. “Transplanting” can used instead of grafting.

### Paramyxovirus Fusion Proteins

In some embodiments, the mutations disclosed herein can be applied to fusion proteins of other Paramyxoviruses. In some embodiments, the mutations disclosed herein can be applied to fusion proteins in other viruses in the Henipavirus genus (in the family Paramyxoviridae, order Mononegavirales). In some embodiments, the mutations disclosed herein can be apply to fusion proteins in Langya virus (LayV), Nipah virus (NiV), Hendra virus (HeV), and other Henipaviruses that infect humans. In some embodiments, the mutations disclosed herein can be applied to fusion proteins in Mojiang virus (MojV), Gamak virus (GakV), Daeryong virus (DarV), and other Henipaviruses that infect animals. Henipaviruses also include Cedar virus (CedV), Ghana virus (GhV), Melian virus (MelV), Denwin virus (DewV) and others. New Henipaviruses are still being discovered. The mutations disclosed herein can be applied to fusion proteins in yet to be discovered Henipaviruses. In some embodiments, the disclosed mutations can be applied to Fusion proteins that are ectodomains of the proteins, to full-length Fusion proteins, or to any part of the Fusion proteins that contains the ectodomains.

In some embodiments, a Fusion protein from a wild-type Henipavirus is the ectodomain of LayV-F and is 483 amino acids in length.

Langya virus wild-type Fusion protein ectodomain
amino acid sequence
(SEQ ID NO: 1):
MAFLKSAIICYLLFYPHIVKSSLHYDSLSKVGIIKGLTYNYKIKGSPSTK
LMVVKLIPNIDGVRNCTQKQFDEYKNLVKNVLEPVKLALNAMLDNVKSGN
NKYRFAGAIMAGVALGVATAATVTAGIALHRSNENAQAIANMKNAIQNTN
EAVKQLQLANKQTLAVIDTIRGEINNNIIPVINQLSCDTIGLSVGIKLTQ
YYSEILTAFGPALQNPVNTRITIQAISSVFNRNFDELLKIMGYTSGDLYE
ILHSGLIRGNIIDVDVEAGYIALEIEFPNLTLVPNAVVQELMPISYNVDG
DEWVTLVPRFVLTRTTLLSNIDTSRCTVTESSVICDNDYALPMSYELIGC
LQGDTSKCAREKVVSSYVPRFALSDGLVYANCLNTICRCMDTDTPISQSL
GTTVSLLDNKKCLVYQVGDILISVGSYLGEGEYSADNVELGPPVVIDKID
IGNQLAGINQTLQNAEDYIEKSEEFLKGINPSI

In some embodiments, the Langya virus wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 1.

In some embodiments, some amino acids in the sequence above can be substituted by other amino acids. In some embodiments, the amino acids that can be substituted include K55, G99, 1109, A111, E134, N150, 1167, D168, R171, S186, A340, S356, V364, S365, S423 and/or L440.

In some embodiments, the amino acids in the paragraph above can be substituted with other amino acids as follows: K55I, G99C, 1109C, A111C, E134P, N150P, I167F, D168A, R171I, S186P, A340C, S356C, V364C, S365C, S423C and/or L440C.

In some embodiments, the substituting amino acids in the paragraph above can be replaced by conservatively substituted amino acids.

Without being held to any particular theory, in some embodiments, a LayV-F can contain 1, 2, 3, 4, 5, 6, 7, 8, 9, 10 or more of the substituted amino acids noted above, in any combination. In some embodiments, the substituted amino acids can be as follows:
a) mutation of glutamic acid 134 to proline to stabilize a kinked conformation;
b) mutation of alanine 340 to cysteine and/or valine 364 to cysteine to stabilize the DII domain;
c) mutation of glycine 99 to cysteine, isoleucine 109 to cysteine, isoleucine 167 to phenylalanine and/or serine 186 to proline;
d) mutation of alanine 111 to cysteine and/or serine 423 to cysteine to stabilize the DII domain;
e) mutation of asparagine 150 to proline to straighten and inhibit the inhibit the HRA helix region;
f) mutation of charged amino acids to non-charged amino acids, lysine 55 to isoleucine, aspartic acid 168 to alanine, and/or arginine 171 to isoleucine to stabilize the prefusion HRA domain conformation;
g) mutation of serine 356 to cysteine and/or lysine 440 to cysteine to stabilize the HRB domain;
h) combinations of the mutations of a) and f); and
i) combinations of the mutations of b) and g).

In some embodiments, these mutations can be combined with other mutations in a fusion protein.

In some embodiments, the Langya virus wild-type Fusion protein ectodomain amino acid sequence can have N133P (SEQ ID NO: 65), S132P (SEQ ID NO: 66), R131P (SEQ ID NO: 67), N135P (SEQ ID NO: 68), A136P (SEQ ID NO: 69), Q137P (SEQ ID NO: 70), N133P and E134P (SEQ ID NO: 71), N133P and N135P (SEQ ID NO: 72), E134P and N135P (SEQ ID NO: 73), N144P (SEQ ID NO: 74), or N144P and A145P (SEQ ID NO: 75). In some embodiments, the Langya virus Fusion protein ectodomain amino acid sequence can have combinations of those amino acid substitutions. In some embodiments, the Langya virus Fusion protein ectodomain amino acid sequence can have one or more of those amino acid substitutions in combination with any other amino acid substitutions disclosed herein.

In some embodiments, the nucleotide sequence encoding the wild-type ectodomain amino acid sequence above can be SEQ ID NO: 33, below:

ATGGCCTTTCTGAAGTCCGCCATCATCTGCTACCTGCTGTTCTACCCTCA
CATCGTGAAGTCCAGCCTGCACTACGACAGCCTGAGCAAAGTGGGCATCA
TCAAGGGCCTGACCTACAACTACAAGATCAAGGGCAGCCCCAGCACCAAG
CTGATGGTGGTCAAGCTGATCCCCAACATCGACGGCGTGCGGAACTGTAC
CCAGAAGCAGTTCGACGAGTACAAGAACCTGGTCAAGAACGTGCTGGAAC
CCGTGAAGCTGGCCCTGAACGCCATGCTGGACAATGTGAAGTCCGGCAAC
AACAAGTACAGATTCGCTGGCGCCATCATGGCCGGCGTTGCACTTGGAGT
TGCCACAGCCGCTACAGTGACAGCCGGAATTGCCCTGCACAGAAGCAACG
AGAACGCCCAGGCCATTGCCAACATGAAGAACGCCATCCAGAACACCAAC
GAGGCCGTGAAGCAGCTCCAGCTGGCCAACAAACAGACCCTGGCCGTGAT
CGACACCATCCGGGGAGAGATCAACAACAACATCATCCCCGTGATCAACC
AGCTGAGCTGTGACACCATCGGCCTGAGCGTGGGAATCAAGCTGACCCAG
TACTACAGCGAGATCCTGACCGCCTTCGGACCCGCTCTGCAGAACCCTGT
GAACACCAGAATCACCATCCAGGCCATCAGCAGCGTGTTCAACCGGAACT
TCGACGAGCTGCTGAAGATCATGGGCTACACCAGCGGCGACCTGTATGAG
ATCCTGCACAGCGGCCTGATCCGGGGCAATATCATCGACGTGGACGTGGA
AGCCGGCTATATCGCCCTGGAAATCGAGTTCCCCAACCTGACACTGGTGC
CCAACGCCGTGGTGCAAGAGCTGATGCCCATCTCCTACAACGTGGACGGC
GACGAGTGGGTTACCCTGGTGCCTAGATTCGTGCTGACCCGGACAACCCT
GCTGAGCAACATCGATACCAGCAGATGCACCGTGACCGAGAGCAGCGTGA
TCTGCGACAACGATTACGCCCTGCCTATGAGCTACGAGCTGATCGGATGT
CTGCAGGGCGACACCAGCAAGTGCGCCAGAGAAAAGGTGGTGTCCAGCTA
CGTGCCCAGATTCGCCCTGTCTGACGGACTGGTGTACGCCAACTGCCTGA
ACACCATCTGCCGGTGCATGGACACCGACACACCTATCTCTCAGAGCCTG
GGCACCACCGTGTCTCTGCTGGATAACAAGAAATGCCTGGTGTACCAAGT
GGGCGACATCCTGATCTCCGTGGGCTCTTATCTCGGCGAGGGCGAGTACA
GCGCCGACAATGTTGAACTGGGCCCTCCTGTGGTCATCGACAAGATCGAC
ATCGGCAATCAGCTGGCCGGCATCAATCAGACCCTGCAGAACGCCGAGGA
CTACATCGAGAAGTCTGAAGAGTTCCTGAAGGGCATCAACCCCAGCATC

In some embodiments, the nucleotide sequence above, encoding the wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 33.

In some embodiments, a Fusion protein from a wild-type Henipavirus is the ectodomain of NiV-F and is 488 amino acids in length.

Nipah virus wild-type Fusion protein ectodomain
amino acid sequence
(SEQ ID NO: 2):
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSL

In some embodiments, the Nipah virus wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 2.

In some embodiments, some amino acids in the sequence above can be substituted by other amino acids. In some embodiments, the amino acids that can be substituted include K60, A116, K139, N155, T173, Q176, A345, E361, V369, S428 and/or I445. In some embodiments, the amino acids that can be substituted can be K139 and/or N155.

In some embodiments, the amino acids in the paragraph above can be substituted with other amino acids as follows: K60I, A116C, K139P, N155P, T173A, Q176I, A345C, E361I or E361C, V369C, S428C and/or I445C. In some embodiments, the amino acids in the paragraph above can be substituted with other amino acids as follows: K139P and/or N155P.

In some embodiments, the substituting amino acids in the paragraph above can be replaced by conservatively substituted amino acids.

Without being held to any particular theory, in some embodiments, a NiV-F can contain 1, 2, 3, 4, 5, 6, 7, 8, 9, 10 or more of the substituted amino acids noted above, in any combination. In some embodiments, these mutations can be combined with other mutations in a fusion protein.

In some embodiments, the Nipah virus wild-type Fusion protein ectodomain amino acid sequence can have M138P (SEQ ID NO: 76), A137P (SEQ ID NO: 77), E136P (SEQ ID NO: 78), N140P (SEQ ID NO: 79), A141P (SEQ ID NO: 80), D142P (SEQ ID NO: 81), M138P and K139P (SEQ ID NO: 82), M138P and N140P (SEQ ID NO: 83), K139P and N140P (SEQ ID NO: 84), S149P (SEQ ID NO: 85), and S149P and S150P (SEQ ID NO: 86). In some embodiments, the Nipah virus Fusion protein ectodomain amino acid sequence can have combinations of those amino acid substitutions. In some embodiments, the Nipah virus Fusion protein ectodomain amino acid sequence can have one or more of those amino acid substitutions in combination with any other amino acid substitutions disclosed herein.

In some embodiments, the nucleotide sequence encoding the wild-type ectodomain amino acid sequence above can be SEQ ID NO: 34, below:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACGCCACCCCCATGACCAACAACATGAGGG
AGTGCCTGACCGGCAGCACCGAGAAGTGCCCCAGGGAGCTGGTGGTGAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCATCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG

In some embodiments, the nucleotide sequence above, encoding the wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 34.

In some embodiments, a Fusion protein from a wild-type Henipavirus is the ectodomain of HeV-F and is 488 amino acids in length.

Hendra virus Fusion protein ectodomain amino acid
sequence (SEQ ID NO: 3):
MATQEVRLKCLLCGIIVLVLSLEGLGILHYEKLSKIGLVKGITRKYKIKS
NPLTKDIVIKMIPNVSNVSKCTGTVMENYKSRLTGILSPIKGAIELYNNN
THDLVGDVKLAGVVMAGIAIGIATAAQITAGVALYEAMKNADNINKLKSS
IESTNEAVVKLQETAEKTVYVLTALQDYINTNLVPTIDQISCKQTELALD
LALSKYLSDLLFVFGPNLQDPVSNSMTIQAISQAFGGNYETLLRTLGYAT
EDFDDLLESDSIAGQIVYVDLSSYYIIVRVYFPILTEIQQAYVQELLPVS
FNNDNSEWISIVPNFVLIRNTLISNIEVKYCLITKKSVICNQDYATPMTA
SVRECLTGSTDKCPRELVVSSHVPRFALSGGVLFANCISVTCQCQTTGR
AISQSGEQTLLMIDNTTCTTVVLGNIIISLGKYLGSINYNSESIAVGPPV
YTDKVDISSQISSMNQSLQQSKDYIKEAQKILDTVNPSL

In some embodiments, the Hendra virus wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 3.

In some embodiments, some amino acids in the sequence above can be substituted by other amino acids. In some embodiments, the amino acids that can be substituted include K60, L104, V114, A116, K139, N155, L172, T173, Q176, S191, A345, D361, V369, S428 and/or V445.

In some embodiments, the amino acids in the paragraph above can be substituted with other amino acids as follows: K60I, L104C, V114C, A116C, K139P, N155P, L172F, T173A, Q176I, S191P, A345C, D361C, V369C, S428C and/or V445C.

In some embodiments, the substituting amino acids in the paragraph above can be replaced by conservatively substituted amino acids.

Without being held to any particular theory, in some embodiments, a HeV-F can contain 1, 2, 3, 4, 5, 6, 7, 8, 9, 10 or more of the substituted amino acids noted above, in any combination. In some embodiments, these mutations can be combined with other mutations in a fusion protein.

In some embodiments, the nucleotide sequence encoding the wild-type ectodomain amino acid sequence above can be SEQ ID NO: 35, below:

ATGGCCACACAAGAAGTGCGGCTGAAGTGCCTGCTGTGCGGCATCATTGT
GCTGGTGCTGTCTCTGGAAGGCCTGGGCATCCTGCACTACGAGAAGCTGTCTAAGAT
CGGCCTGGTCAAGGGCATCACCCGGAAGTACAAGATCAAGAGCAACCCTCTGACCA
AGGACATCGTGATCAAGATGATCCCCAACGTGTCCAATGTGTCCAAGTGCACCGGC
ACCGTGATGGAAAACTACAAGAGCCGGCTGACCGGCATTCTGAGCCCTATCAAGGG
CGCCATCGAGCTGTACAACAACAACACCCACGACCTCGTGGGCGACGTGAAACTTG
CTGGCGTTGTGATGGCCGGAATCGCCATCGGAATTGCCACAGCCGCTCAGATTACAG
CTGGCGTGGCCCTGTATGAGGCCATGAAGAACGCCGACAACATCAACAAGCTGAAG
TCCAGCATCGAGAGCACCAACGAGGCCGTGGTCAAGCTGCAAGAGACAGCCGAGA
AAACCGTGTACGTGCTGACAGCCCTGCAGGACTACATCAACACCAACCTGGTGCCT
ACCATCGACCAGATCTCTTGCAAGCAGACCGAGCTGGCCCTGGATCTGGCCCTGAGC
AAGTACCTGAGCGACCTGCTGTTCGTGTTCGGCCCCAATCTGCAGGACCCTGTGTCC
AACAGCATGACCATCCAGGCCATCAGCCAGGCCTTTGGCGGCAACTATGAGACACT
GCTGAGAACCCTGGGCTACGCCACCGAGGACTTCGACGATCTGCTGGAAAGCGACT
CTATCGCCGGCCAGATCGTGTATGTGGACCTGAGCAGCTACTACATCATCGTGCGGG
TGTACTTCCCCATCCTGACCGAGATCCAGCAGGCCTACGTGCAAGAGCTGCTGCCCG
TGTCCTTCAACAACGACAACAGCGAGTGGATCAGCATCGTGCCCAACTTCGTGCTGA
TCCGGAACACCCTGATCAGCAACATCGAAGTGAAGTACTGCCTGATCACCAAGAAG
TCCGTCATCTGCAACCAGGACTACGCCACACCTATGACCGCCAGCGTCAGAGAGTGT
CTGACAGGCAGCACCGACAAGTGCCCCAGAGAACTGGTGGTGTCCAGCCACGTGCC
AAGATTTGCTCTGTCTGGCGGCGTGCTGTTCGCCAACTGTATCAGCGTGACCTGCCA
GTGCCAGACCACAGGCAGAGCCATTTCTCAGAGCGGCGAACAGACCCTGCTGATGA
TCGACAACACCACCTGTACCACCGTGGTGCTGGGCAACATCATCATCAGCCTGGGCA
AGTATCTGGGCAGCATCAACTACAACTCCGAGAGCATTGCCGTGGGACCTCCAGTGT
ACACCGATAAGGTGGACATCAGCAGCCAGATCAGCAGCATGAACCAGAGCCTGCAG
CAGAGCAAGGATTACATCAAAGAGGCCCAGAAAATCCTGGACACCGTGAATCCCAG
CCTC

In some embodiments, the nucleotide sequence above, encoding the wild-type Fusion protein ectodomain amino acid sequence can be 80, 81, 82, 83, 84, 85, 86, 87, 88, 89, 90, 91, 92, 93, 94, 95, 96, 97 98 or 99 percent identical to SEQ ID NO: 35.

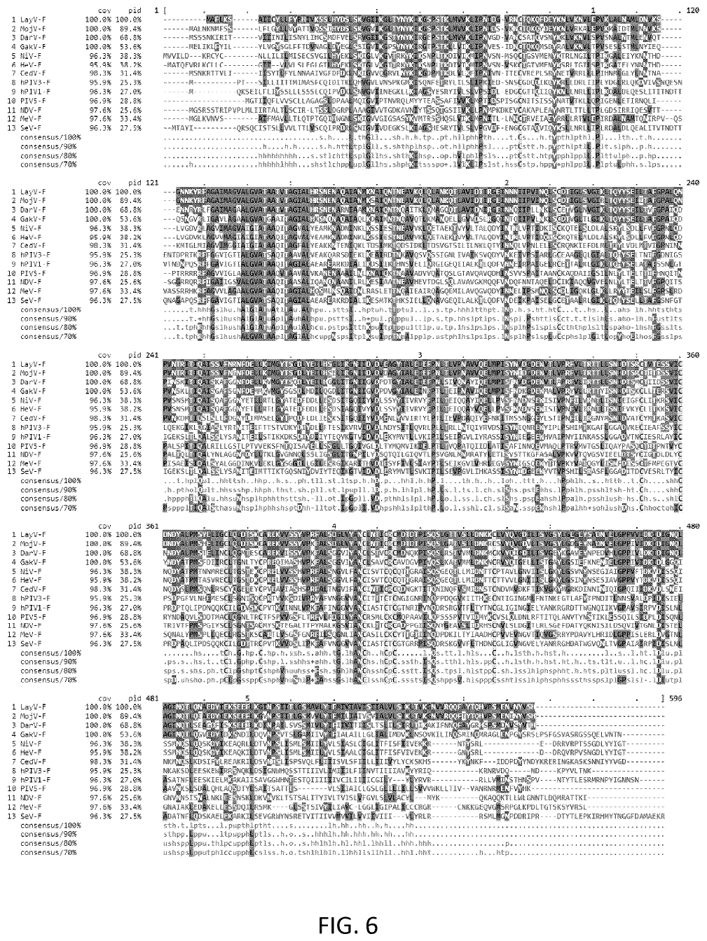

One of skill in the art will recognize, that for proteins with a high level of identity to one another, mutations in specific amino acids in one protein, that result in an improved or different property or function of the protein, can be found that affect a related protein in the same way. For example, LayV-F (483 amino acids), NiV-F (488 amino acids; 96.3% identical to LayV) and HeV-F (488 amino acids; 95.9% identical to LayV-F) are related as shown in the alignments of FIG. 6. Mutations in one of these three, and other related proteins, can have functionally analogous mutations in related proteins.

In some embodiments, E134 in LayV-F can be analogous to E139 in NiV and in HeV. A340C, V364C in LayV-F can be analogous to A345C, V369C in NiV and HeV. The concept is that, in some embodiments, although the mutations disclosed herein can be made in LayV-F, those mutations can be applied to NiV-F and/or HeV-F. In some embodiments, the mutations made in LayV—F, NiV—F and/or HeV—F can be applied to other related (e.g., Henipavirus, Paramyxovirus) fusion proteins.

### Epitopes

As used herein, the term “epitope” can include any protein determinant capable of specific binding to an immunoglobulin, a scFv, or a T-cell receptor. The variable region, of an antibody for example, allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can have chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

### Antibodies

An “antibody” or “antigen-binding polypeptide” can refer to a polypeptide or a polypeptide complex that specifically recognizes and binds to an antigen, such as fusion peptide. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment” can refer to a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)2, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In embodiments the regions are connected with a short linker peptide, such as a short linker peptide of about ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglubulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (Y, u, a, 8, 8) with some subclasses among them (e.g., γ1-γ4). Certain classes have subclasses as well, such as IgG1, IgG2, IgG3 and IgG4 and others. The immunoglobulin subclasses (isotypes) e.g., IgG1, IgG2, IgG3, IgG4, IgG5, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions can adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” can refer to the numbering system set forth by Kabat et al., U.S. Dept. of Health and Human Services, “Sequence of Proteins of Immunological Interest” (1983).

Antibodies may be polyclonal or monoclonal; xenogeneic, allogeneic, or syngeneic; or modified forms thereof (for example, humanized, chimeric, etc.). Antibodies may also be fully human. Preferably, antibodies of the invention bind specifically or substantially specifically to a component of cBAF complex. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

### Methods—Immunization

The immune response against the compositions of the invention can be generated by one or more inoculations of a subject with an immunogenic composition of the invention. A first inoculation can be termed a “primary inoculation” and subsequent immunizations can be termed “booster inoculations”. Booster inoculations can enhance the immune response, and immunization regimens including at least one booster inoculation can be used. Any composition of the invention may be used for a primary or booster immunization. The adequacy of the vaccination parameters chosen, e.g., formulation, dose, regimen and the like, can be determined by taking aliquots of serum from the subject and assaying antibody titers during the course of the immunization program. Alternatively, T cell populations can by monitored by conventional methods. The clinical condition of a subject can be monitored for the desired effect, e.g., limiting Paramyxovirus and/or Henipavirus infection, improvement in disease state (e.g., reduction in viral load), etc. If such monitoring indicates that vaccination is sub-optimal, the subject can be boosted with an additional dose of composition, and the vaccination parameters can be modified in a fashion expected to potentiate the immune response. Thus, for example, the dose of the polypeptide, VLP, or composition, and/or adjuvant, can be increased or the route of administration can be changed.

As described herein, aspects of the invention are drawn to compositions and methods of preventing a viral infection in a subject.

Aspects of the invention are drawn to compositions and methods of treating a viral infection in a subject.

For example, one or more of the following effects can result from the administration of a therapy or a combination of therapies as described herein: (i) the reduction or amelioration of the severity of a viral infection and/or a symptom associated therewith; (ii) the reduction in the duration of a viral infection and/or a symptom associated therewith; (iii) the regression of a viral infection and/or a symptom associated therewith; (iv) the reduction of the titer of a virus; (v) the reduction in organ reduced function or failure associated with a viral infection; (vi) the reduction in hospitalization of a subject; (vii) the reduction in hospitalization length; (viii) the increase in the survival of a subject; (ix) the elimination of a virus infection; (x) the inhibition of the progression of a viral infection and/or a symptom associated therewith; (xi) the prevention of the spread of a virus from a cell, tissue or subject to another cell, tissue or subject; and/or (xii) the enhancement or improvement the therapeutic effect of another therapy.

In embodiments, a therapeutically effective amount can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments, the therapeutically effective amount comprises less than about 0.1 mg, about 0.1 mg, about 0.5 mg, about 1.0 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 70 mg, about 80 mg, about 90 mg, about 100 mg, about 120 mg, about 135 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 1.0 g, about 1.5 g, about 2.0 g, about 2.5 g, about 5 g, about 10 g, about 25 g, about 50 g, or more than 50 g.

Aspects of the invention are also drawn to managing a subject afflicted with or at risk of a viral infection.

Aspects of the invention can comprise administering to a subject an immunogenic composition comprising an epitope from a mutant Henipavirus fusion protein antigen.

In embodiments, administering can refer to providing a therapeutically effective amount of the composition to a subject. The formulation or pharmaceutical compound can be administered alone, but can be administered with other compounds, excipients, fillers, binders, carriers or other vehicles selected based upon the chosen route of administration and standard pharmaceutical practice. In some embodiments, an immune response can be stimulated in an individual by administering to the individual the immunogenic composition described herein or the amino acid epitope grafted into the scaffold protein described herein. In embodiments, the method is for eliciting an immune response to a fusion peptide epitope and/or a mutant Henipavirus fusion protein antigen.

Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments, the compound can be administered alone, or can be administered as a pharmaceutical composition together with other compounds, excipients, carriers, diluents, fillers, binders, or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Embodiments can be administered to a subject in one or more doses. The dose level can vary as a function of the specific composition or pharmaceutical composition administered, the severity of the symptoms and the susceptibility of the subject to side effects. Dosages for a given compound are readily determinable by a variety of means. For example, dosages can be determined by standard clinical techniques. In addition, in vitro or in vivo assays can be employed to help identify optimal dosage ranges. The precise dose to be employed can also depend on the route of administration and can be decided according to the judgment of the practitioner and each patient's circumstances.

In some embodiments, multiple doses of the composition can be administered. The frequency of administration and the duration of administration of the composition can vary depending on any of a variety of factors, e.g., patient response, severity of the symptoms, and the like. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. In an embodiment, the pharmaceutical composition can be administered 1 to 4 times a day over a period of time, such as 1 to 10-day time period, or longer than a 10-day period of time.

In embodiments, the composition can be administered in combination with one or more additional active agents. For example, a first agent (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent (e.g., a prophylactic or therapeutic agent) to a subject with a disease or disorder or a symptom thereof.

Embodiments as described herein further comprises administering one or more additional active agents to a subject together with a mutant Henipavirus fusion protein antigen. Non-limiting examples of such additional active agents can comprise an anti-viral agent (e.g., remdesivir, molunpiravir, paxlovid, or any combination thereof), a vaccine, an anti-inflammatory agent, a pain reliever, a steroid, or any combination thereof.

### Methods—Protein Analysis

Detection and analysis of protein conformational changes, for example, pre-fusion to post-fusion conformational changes of viral fusion proteins, can involve significant work and is difficult to perform in a high-throughput manner. Disclosed herein are techniques that can rapidly identify conformational shifts of fusion proteins. In some embodiments, the methods disclosed herein can be used for fusion proteins that undergo conformation changes when heated. In some embodiments, the methods disclosed herein can be used for viral fusion proteins that undergo shifts from pre-fusion to post-fusion conformations when heated. The disclosed studies (Examples 11 and 12) used Henipavirus fusion proteins.

In some embodiments, differential scanning fluorimetry (DSF) can be used to detect and analyze conformational changes in fusion proteins, including viral fusion proteins (Example 11). In some embodiments, changes in a DSF tracing can be detected when a wild-type fusion protein transitions from a pre-fusion to post-fusion conformation during heating. In some embodiments, variant or mutant fusion proteins that do not transition or have altered transitioning from a pre-fusion to a post-fusion conformation can have a different DSF tracing profile than the wild-type fusion protein. These DSF profile changes can be used to screen fusion protein mutants, for example, for those that do not transition or do not transition normally from a pre-fusion state to a post-fusion state, or that are stabilized or locked in a pre-fusion or post-fusion state.

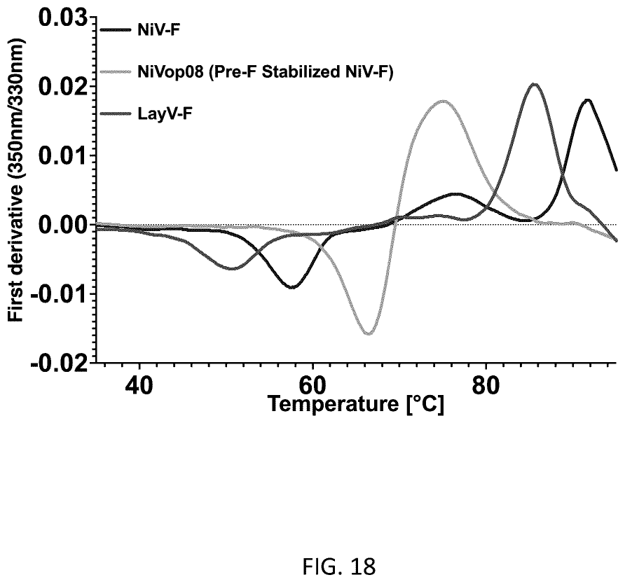

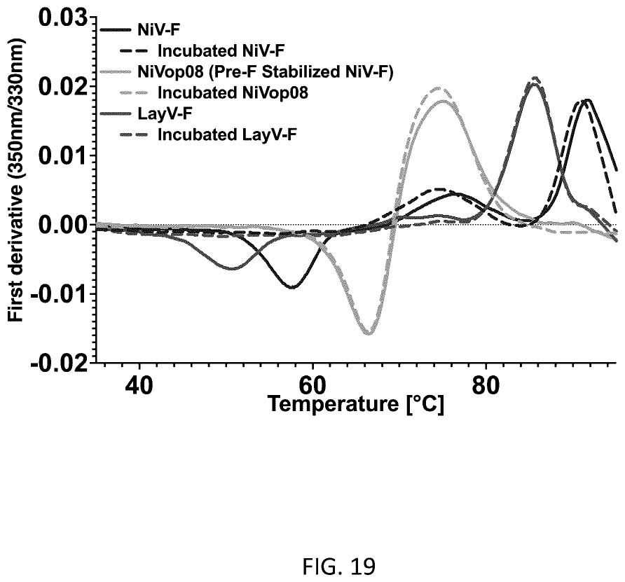

In some embodiments, a DSF tracing of a wild-type fusion protein can have a “negative” peak (e.g., below 0.00 on a DSF tracing plot of first derivative of wavelength (e.g, 350 nm/330 nm) vs. temperature; FIG. 18). In some embodiments, the negative peak occur between about 40-90° C., 45-85° C., 50-80° C., 55-75° C. or 45-65° C. on the temperature axis of a DSF tracing. In some embodiments, a fusion protein that is stabilized or locked in a pre-fusion conformation may not display the negative peak on a DSF tracing (FIG. 19). In some embodiments, fusion proteins in which mutations have been made (e.g., amino acid substitutions) can be screened for those mutant proteins that do not have the negative peak. In some embodiments, a tracing for a fusion protein can be compared to a tracing for the same protein, except that the same protein has been pre-heated (e.g., to a temperature that results in pre-fusion to post-fusion transition).

In some embodiments, differential binding of a protein by an antibody, where the antibody binding is dependent upon conformation state of the protein, can be used to detect and analyze conformational changes in fusion proteins, including viral fusion proteins. In some embodiments, bio-layer interferometry (BLI) can be used for this purpose (Example 12).

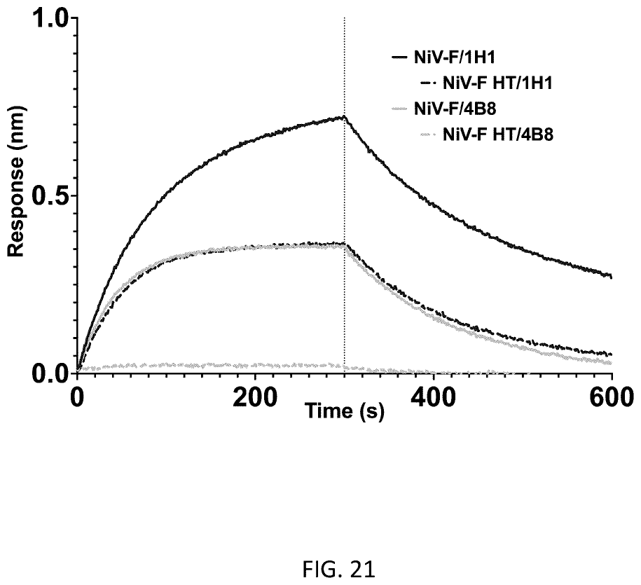

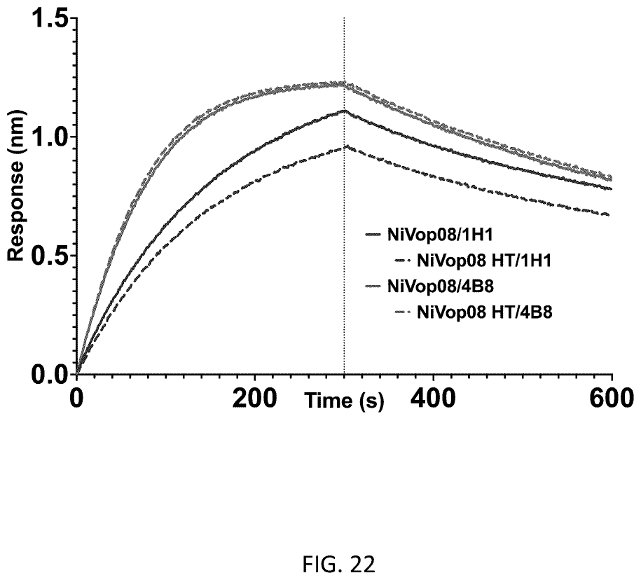

Herein, an antibody can bind to both a pre-fusion and post-fusion form of a viral fusion protein, can bind to the pre-fusion but not the post-fusion form of the fusion protein, or can bind to the post-fusion form but not the pre-fusion form of the fusion protein. In some embodiments, an antibody that differentially binds to one or the other of the pre-fusion or post-fusion form of a viral fusion protein can be used to screen mutants of fusion proteins that have altered transitioning between pre-fusion and post-fusion forms, or that are stabilized or locked in one or the other conformation (FIGS. 21 and 22).

### Kits

Aspects of the invention are also directed towards kits, such as kits comprising compositions as described herein. For example, the kit can comprise prophylactic and/or therapeutic combination compositions described herein.

In one embodiment, the kit includes (a) a container that contains a composition, such as that described herein, and optionally (b) informational material. The informational material can be descriptive, instructional, marketing or other material that relates to the methods described herein and/or the use of the agents for therapeutic benefit.

In an embodiment, the kit includes two or more agents. For example, the kit can include a container comprising an immunogenic composition comprising an epitope from mutant Henipavirus fusion protein antigen, and a second container comprising a second active agent.

In embodiments, the kit further comprises a third container comprising a third active agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The immunogenic composition can be provided in any form, e.g., liquid, dried or lyophilized form, or for example, substantially pure and/or sterile. When the agents are provided in a liquid solution, the liquid solution is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

The Examples/Methods have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

Data Availability. Cryo-EM reconstructions and atomic models generated during this study are available at wwPDB and EMBD (rcsb.org; http://emsearch.rutgers.edu) under the accession codes PDB IDs 8FEJ and 8FEL and EMDB IDs EMD-29029 and EMD-29032.

Plasmids. Gene synthesis for all plasmids generated by this study were performed and the sequence confirmed by GeneImmune Biotechnology (Rockville, MD). The fusion protein ectodomain constructs included F protein residues 1 to 438 (GenBank: UUV47205.1), a C-terminal T4 fibritin trimerization motif (FOLDON), a C-terminal HRV3C protease cleavage site, a TwinStrepTag and an 8×HisTag. The ectodomain was cloned into the mammalian expression vector paH. Synthetic heavy and light chain variable domain genes for Fabs were cloned into a modified pVRC8400 expression vector, as previously described (5, 24, 25). Antibody variable regions for both heavy and light chain replaced the variable regions from the VRC01 antibody plasmid (52), with the IL-2 secretion signal and human kappa constant domain being used for light chain plasmids.

Cell culture and protein expression. For F ectodomains, GIBCO FreeStyle 293-F cells (embryonal, human kidney) were maintained at 37° C. and 9% CO2 in a 75% humidified atmosphere in FreeStyle 293 Expression Medium (GIBCO). The plasmid was transiently transfected using Turbo293 (SpeedBiosystems) and incubated at 37° C., 9% CO2, 75% humidity with agitation at 120 rpm for 6 days. On the day following transfection, HyClone CDM4HEK293 media (Cytiva, MA) was added to the cells. For antibodies, GIBCO Expi293F cells (embryonal, human kidney) were maintained at 37° C. and 9% CO2 in a 75% humidified atmosphere in Expi293 Expression Medium (GIBCO). The Plasmid was transiently transfected using Expifectamine 293 (GIBCO) and incubated at 37° C., 9% CO2, 75% humidity with agitation at 120 rpm for 6 days. On the day following transfection, Expifectamine 293 Enhancers (GIBCO) were added to the cells.

Protein purification. On the 6th day post transfection, the fusion protein ectodomains were harvested from the concentrated supernatant, purified using StrepTactin resin (IBA LifeSciences) and size exclusion chromatography (SEC) using a Superose 6 10/300 GL Increase column (Cytiva, MA) equilibrated in PBS buffer (Thermo Scientific 137 mM NaCl, 2.7 mM KCl, pH 7.4, 10 mM Phosphate Buffer, 1.8 mM Potassium Phosphate Monobasic). Antibodies were purified using Protein A affinity (Thermo Scientific) and SEC using a Superose 6 10/300 GL Increase column (Cytiva, MA) equilibrated in PBS buffer (Thermo Scientific 137 mM NaCl, 2.7 mM KCl, pH 7.4, 10 mM Phosphate Buffer, 1.8 mM Potassium Phosphate Monobasic). All steps of the purification were performed at room temperature and in a single day. Protein quality was assessed by SDS-Page using NuPage 4-12% (Invitrogen, CA). The purified proteins were flash frozen and stored at −80° C. in single-use aliquots. Each aliquot was thawed by a 5-minute incubation at 37° C. before use.

Cryo-EM. Purified ectodomain was diluted to a concentration of 1.5 mg/mL in PBS (described above) with 0.0005% DDS and 0.38% glycerol added. A 2.2 μL drop of protein was deposited on a Quantifoil R1.2/1.3 grid (Electron Microscopy Sciences, PA) that had been glow discharged for 10 seconds using a PELCO easiGlow™ Glow Discharge Cleaning System. After a 30-second incubation in >95% humidity, excess protein was blotted away for 2.5 seconds before being plunge frozen into liquid ethane using a Leica EM GP2 plunge freezer (Leica Microsystems). Frozen grids were imaged using a Titan Krios (Thermo Fisher) equipped with a K3 detector (Gatan). Movie frame alignment was carried out using UNBLUR (53). The cryoSPARC (12) software was used for data processing. Phenix (54), PyMOL (55), ChimeraX (56) and Isolde (57) were used for model building and refinement. Phenix was first used to fit the initial models into maps. ISOLDE was used to manually adjust residues to address rotamer and Ramachandran outliers. Phenix was again used to perform real-space refinement, energy-minimization of side-chain positions, and to set B-factors during an ADP-only real-space refinement.

SDS-PAGE. Prepared 1, 3, or 8 μg of sample with Laemmli sample buffer (BioRad), PBS buffer, and with or without 300 mM DTT (Reduced/Non-Reduced). Loaded to NuPAGE 4-12% Bis-Tris gel (Invitrogen) and ran at 175V with MES-SDS running buffer until complete. Stained with Coomassie blue (Novex) for 30 minutes before water destain and imaging.

Differential scanning fluorimetry. DSF assay was performed using Tycho NT. 6 (NanoTemper Technologies). Spike ectodomains were diluted to approximately 0.15 mg/mL. Intrinsic fluorescence was measured at 330 nm and 350 nm while the sample was heated from 35 to 95° C. at a rate of 30° C./min. The ratio of fluorescence (350/330 nm) and inflection temperatures (Ti) were calculated by the Tycho NT. 6 apparatus.

In some examples, samples were thawed for 3 minutes at 37° C. and were diluted to 1 mg/mL with PBS. Samples were incubated at 60° C. for 2 hours. Five μL was removed and diluted to 0.2 mg/mL by adding 20 μL of PBS. The remaining 15 μL was transferred for immediate NSEM analysis. Performed standard DSF analysis.

In some examples, a range of temperatures and treatment times was used to obtain conditions capable both of converting to postfusion while not damaging prefusion stabilized candidates. Samples were thawed at ambient temperature or at 37° C. for 3 minutes. Samples were then left to stand at ambient room temperature or laced at at 37° C. in a thermocycler. Tubes from both conditions were taken and prepared for DSF analysis at 15, 30, 60, 90, and 120 minute timepoints. Capillaries (10 μL) of each sample condition and placed into the capillary rack for analysis. Settings were as follows: Intensity, 100%; Gradient, 7.0° C./min; Range, 35.0° C.-95.0° C.; Refolding ramp, Off; DLS, Off.

Difference distance matrices (DDM). DDM plots were generated using the Bio3D package (Grant et al., 2021) implemented in R (R Core Team, 2018. R: A language and environment for statistical computing. R Foundation for Statistical Computing, Vienna, Austria. R-project.org/).

Bio-layer Interferometry (BLI). Antibody binding to Henipavirus F proteins was assessed using BLI on an Octet RED 384 (Sartorius, formerly ForteBio) with Forte Bio Kinetics Buffer. All binding assays were performed at 30° C. Antibodies were immobilized on Anti-Human Fc Capture tips (ForteBio), loaded at 20 μg/mL for 300s. F proteins were used as analytes at 100 nM, with an association time of 480s and dissociation time of 600s. Sensorgram data were reference-subtracted and analyzed using the Octet Analysis Studio software (Sartorius), with a reference tip for each immobilized antibody and reference sample for each analyte.

In some examples, samples were thawed at ambient temperature, diluted and 1×HBS added to make the samples 0.5 mg/ml. The samples were then diluted for heat-treatment and distribution on a BLI plate. Dilution was 1 uL of the 0.5 mg/mL samples to PCR tubes and added 99 μL of 1×HBS. One sample was left at ambient temperature. One sample was placed in a thermocycler at the set temperature and left for 15 minutes. Samples were then distributed to a BLI plate. Steps were as follows: Sensor Check in A1: 120 s; Baseline: 120 s; Associate in B1: 300 s; Dissociate in B1: 300 s. The machine was set to run at 30° C., 3 mm sensor offset, 5 Hz acquisition rate, 10-minute sensor equilibration before start.

In some examples, HNV-F Antigenicity was tested before and after 50° C. Heat Treatment. In some examples, 60° C. for 2 hours can be too harsh and can alter antigenicity of. In some examples, 50° C. for 15 minutes was used to convert F proteins, and to retain antigenicity.

Sequence Alignment. Paramyxovirus F sequences aligned with Clustal Omega tool (58). Output alignment was colored and assigned consensus residue types by MVeiw (1). Sequences obtained from either Uniprot or Genbank. Uniprot accession numbers: NiV-F: Q9IH63, HeV-F: 089342, hPIV3-F: P06828, PIV5-F: P04849, MeV-F (ICB Strain): Q786F3, MojV-F: W8SKT3, CedV-F: J7GX38, hPIV1-F: P12605, NDV-F (Texas Strain): P26628, SeV-F (Z Strain): P04855. Genbank accession numbers: LayV-F: UUV47205.1, GakV-F: QYO90524.1, DarV-F: QYO90531.1.

### Mouse Immunization and Studies.

VRC01ucadKI, hTdT HOM; HOM; and het (SE13) mice were used (Luo, Sai, et al. “Humanized V (D) J-rearranging and TdT-expressing mouse vaccine models with physiological HIV-1 broadly neutralizing antibody precursors.” Proceedings of the National Academy of Sciences 120.1 (2023): e2217883120). These mice can be useful for evaluating vaccination strategies for eliciting bnAbs. Mice were intramuscularly (i.m.) immunized with LayV-F_WT (25 mcg/animal) and NiVop8 (25 mcg/animal) adjuvanted with GLA-SE (IDRI EM-082, 5 mcg/animal) at weeks 0, 4, 15 and 21. Serum titers of antibodies in the mice were monitored by ELISA. Mice with high-binding antibody titers were selected for subsequent spleen cell fusion and B-cell sorting experiments.

Binding ELISAs were conducted by coating 384 well plates (Costar #3700) with 2 mcg/ml Streptavidin (Thermo Fisher Scientific Inc. Cat. No. S-888) in 0.1M sodium bicarbonate overnight at 4 C. Plates were washed (PBS/0.1% Tween-20) and blocked with assay diluent (PBS/4% whey protein/15% Normal Goat Serum/0.5% Tween-20/0.05% Sodium Azide) for one hour. Proteins were added at 2 mcg/ml in assay diluent for 60 minutes followed by washing. Mouse serum samples were incubated on the plates for 90 minutes in 12 by 3-fold serial dilutions starting at 1:30 followed by washing. Goat anti-mouse IgG-HRP secondary antibody (Southern Biotech #1030-05) diluted 1:10,000 in assay diluent without azide was added at 10 μl per well and incubated for 60 minutes. The plates were washed and detected with 20 μl SureBlue Reserve (Seracare #5120-0081) for 15 minutes. The reaction was stopped with the addition of 20 μl HCL stop solution. Plates were read at 450 nm. All steps were done at room temperature unless otherwise noted.

Hybridoma cell line generation and monoclonal antibody production used mice that were boosted with the indicated priming antigen 3 days prior to fusion. Spleen cells were harvested and fused with NS0 murine myeloma cells using PEG1500 to generate hybridomas. After 2 weeks, supernatant of hybridoma clones were collected and screened by binding ELISA.

Hybridomas cells that secreted LayVF antibodies were cloned by limiting dilution until the phenotypes of all limiting dilution wells are identical. IgG mAbs were purified by protein G purification. The VH and VL sequences of mAbs were amplified from hybridoma cell RNA using primers.

### Example 1

### Purification and Structural Determination of Lay V-F

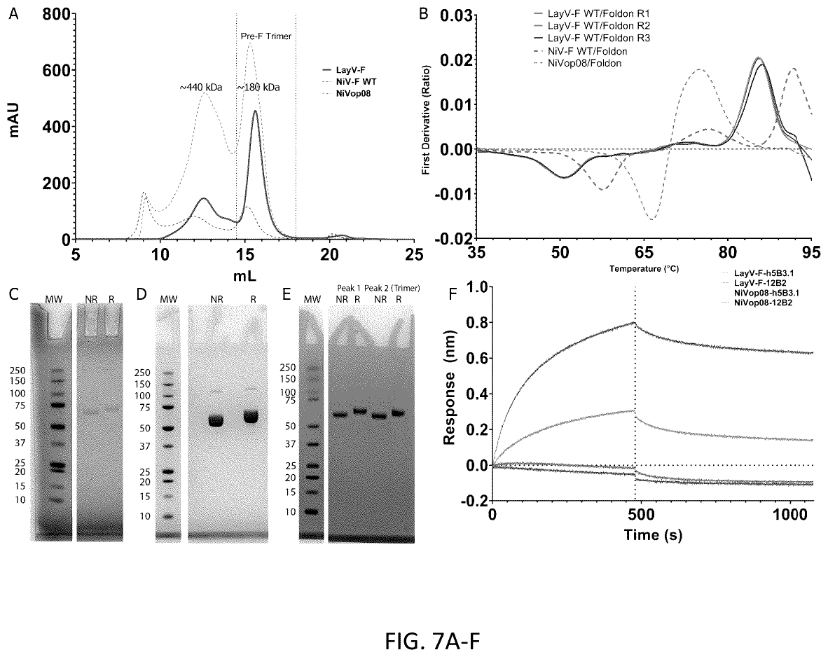

We purified the LayV-F ectodomain based on protocols previously developed for NiV-F, by expressing in 293F cells and purifying via StrepTactin (IBA Lifesciences) affinity chromatography followed by size exclusion chromatography. Like the NiV-F protein, the Lay V F ectodomain elutes with a distinct peak at ˜180 kDa. Higher molecular weight species, broader than the main peak, were also observed at ˜400 kDa and above (FIG. 7A). These could be the result of low-affinity interactions between individual trimers. SDS-PAGE analysis of these high molecular weight species in both NiV-F and LayV-F purifications has consistently produced identical bands corresponding to the ˜180 kDa species, consistent with the uncleaved F protein (FIG. 7E). Higher order multimers of Henipavirus F proteins with rings described as “hexamers of trimers” have been previously reported (2). While larger molecular weight species were detected during purification of LayV-F (FIG. 7A), they comprised a far lower proportion of total protein as compared to purifications of wild-type NiV-F and a prefusion-stabilized NiV-F mutant, NiVop08 (FIG. 7A) (10). The yield of LayV-F at ˜1.2 mg/L was considerably higher than that of wild-type NiV-F at ˜250 μg/L. The NiVop08 yield was higher than the unstabilized NiV-F construct at ˜3 mg/L, consistent with the general trend of prefusion-stabilized fusion protein constructs expressing more efficiently than their wild-type counterparts (35-38). Differential scanning fluorimetry (DSF) indicated reduced stability of LayV-F compared to NiV-F (FIG. 7B). The LayV-F DSF profile closely resembles that of wild-type NiV-F, but is left-shifted, indicating that the temperature-induced conformational changes or unfolding events are occurring at a lower temperature for LayV-F.

We determined the structure of the LayV-F ectodomain trimer using cryo-EM.

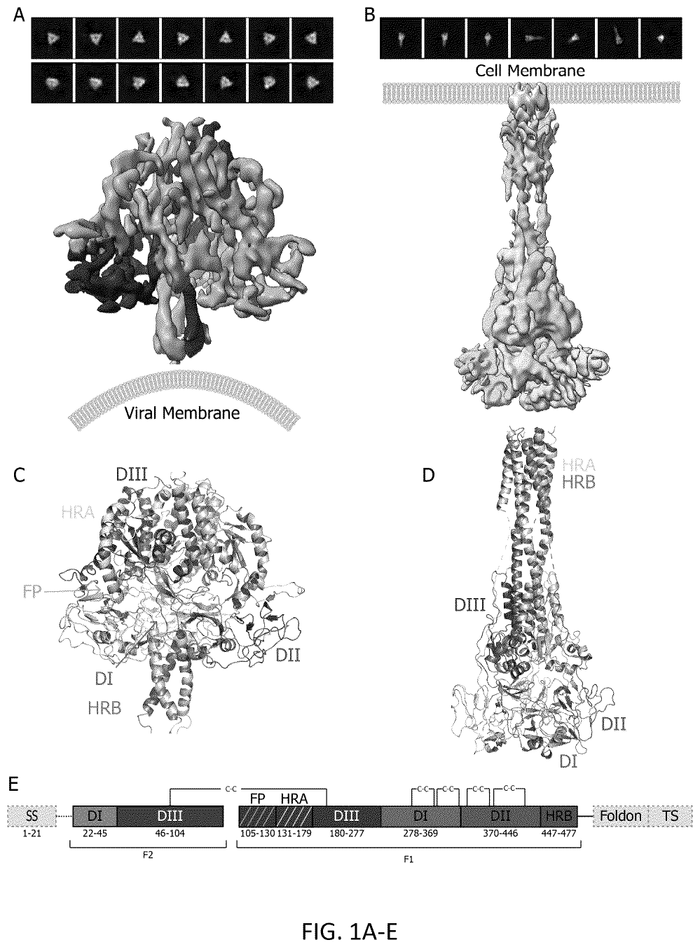

Reference-free 2D classes revealed both pre- and post-fusion conformations of LayV-F in the cryo-EM dataset (FIGS. 1A-B). After several rounds of classification and sorting we obtained 4.64 Å reconstructions (calculated within cryoSparc according to the FSC 0.143 gold-standard criterion) of the pre-fusion and post-fusion forms of the LayV F protein from approximately 200,000 and 80,000 particles, respectively. Assessment of the three-dimensional FSC (3DFSC) plot for these maps indicated resolutions of 5.42 Å and 5.25 Å for the pre- and post-fusion conformations, respectively (39). The structures determined revealed pre- and post-fusion architectures (FIGS. 1A and 1C and 1B and 1D, respectively) that were similar to those determined for other paramyxoviruses (2-4, 8, 40, 41). The pre-fusion LayV F structure has a compact “clove-like” shape with a large internal cavity and spans ˜110 Å from the bottom of the stalk to the top of the apex. In comparison, the post-fusion structure is elongated, spanning ˜160 Å, with regions that formed the pre-fusion apex straightening into a central coiled-coil, ending with the trademark six-helix bundle of the post-fusion conformation.

The LayV F ectodomain includes a fusion peptide (FP) and two heptad repeat sequences that both contribute to three domains, previously described by Yin et al. for the parainfluenzavirus 5 (PIV5) F protein (FIGS. 1C-1E) (41). Domain I (DI) is comprised of the most N-terminal residues after cleavage of the signal peptide, 22-45, and residues 278-369. Its central β-sheet connects Domain II (DII) to the apical Domain III (DIII). DII, from residues 370-446, makes significant contact with the cleavage site and fusion peptide of the neighboring protomer before leading to heptad repeat B (HRB), located at the most C-terminal end of the ectodomain. DIII, from residues 46-277, contains the cleavage site, fusion peptide, and heptad repeat A (HRA) (FIGS. 1C-1E). Residues 93-140 from DIII and 441-450 from DII, which become linker regions leading to the repositioned heptad repeats now forming the six-helix bundle, are not resolved in the post-fusion structure (FIG. 1E).

### Example 2

### Pre- to Post-Fusion Conversion Mechanism of Lay V-F

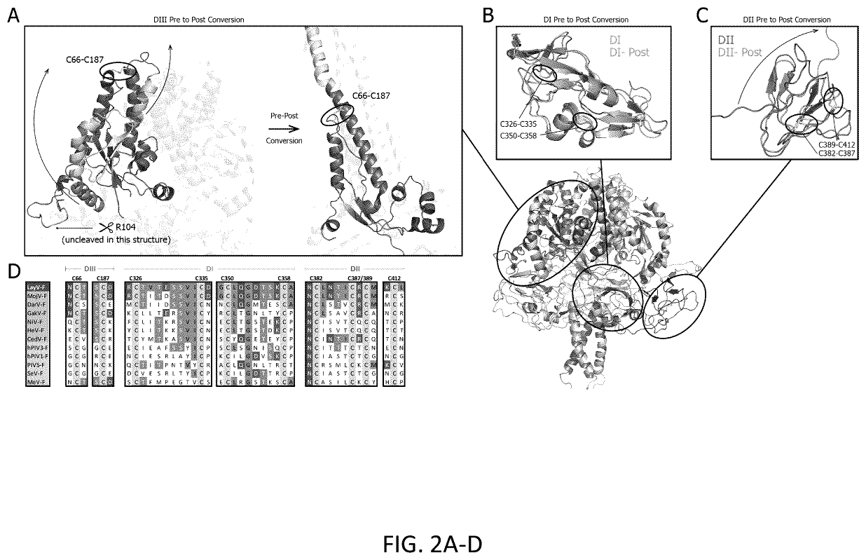

Though the Henipavirus genus was established with the discovery of Hendra virus in 1994 (24, 42), the number of members of the genus has dramatically expanded in recent years with the discovery not only of Langya virus, but also Gamak and Daeryong virus, both identified in 2021 (23). Within the past three years, Henipaviruses have been identified in an expanded range of animal hosts and geographic locations. No longer contained to southeast Asia and Australia, new species have been discovered in Africa and South America, specifically Angavokely virus and Peixe-Boi virus, respectively (43, 44). Although there is very high structural similarity for fusion proteins across not only the Henipavirus genus, but within the larger Paramyxoviridae family, there can be low sequence identity between species, with LayV-F having ˜40% sequence identity with NiV-F, and ˜30% or less identity to other non-Henipavirus paramyxoviruses (FIG. 6). However, with more Henipavirus members identified, some conserved sequence features have become more evident. These include 10 highly conserved cysteines present in the ectodomain of paramyxovirus F proteins (FIG. 2A-2D). Identifying the locations of these cysteines in the pre- and post-fusion LayV-F structures reveals their substantial role in maintaining domain fold integrity during the dramatic conformational changes that occur during fusion.

Despite the different shapes of the pre- and post-fusion conformations of Lay V-F resulting in a 51.6 Å Cα RMSD, superimposition of the DI and DII domains demonstrates that these domains remain largely invariant through the pre- to post-fusion transition of the F protein (FIGS. 2A-2C). Both DI and DII domains have two internal pairs of disulfide bonds that help maintain the rigidity of these domains (FIGS. 2B-2D). DI shows a pre- to post-fusion Ca RMSD of 2.16 Å, while DII undergoes more total movement, resulting in pre- to post-fusion Ca RMSD of 16.5 Å with the linker region from S423 to 1446 that leads to HRB extending away from the domain as HRB moves to its post-fusion location (FIG. 2C). Excluding these residues from the RMSD calculations, DII shifts only 2.3 Å. The difference in conformation of DIII from pre- to post-fusion is more dramatic. Based on sequence alignment, the F1-F2 cleavage site for LayV-F is expected to be immediately after the highly conserved residue R104 (FIG. 6), however cleavage did not occur in this construct (FIG. 7C-7D). The largest positional shifts during the pre- to post-fusion transition occurred in the fusion peptide and HRA, which extend and continue the along the path of the apical helix, now forming the central coiled-coil (FIG. 1B, 1D). These movements occur around a hinge point in DIII where the apical helix is connected to the rest of DIII by a disulfide bond, again demonstrating the role of the conserved cysteines in providing an anchor for large conformational changes in the F protein (FIG. 2A).

### Example 3

### Fusion Triggering Mechanism of Henipavirus Fusion Proteins.

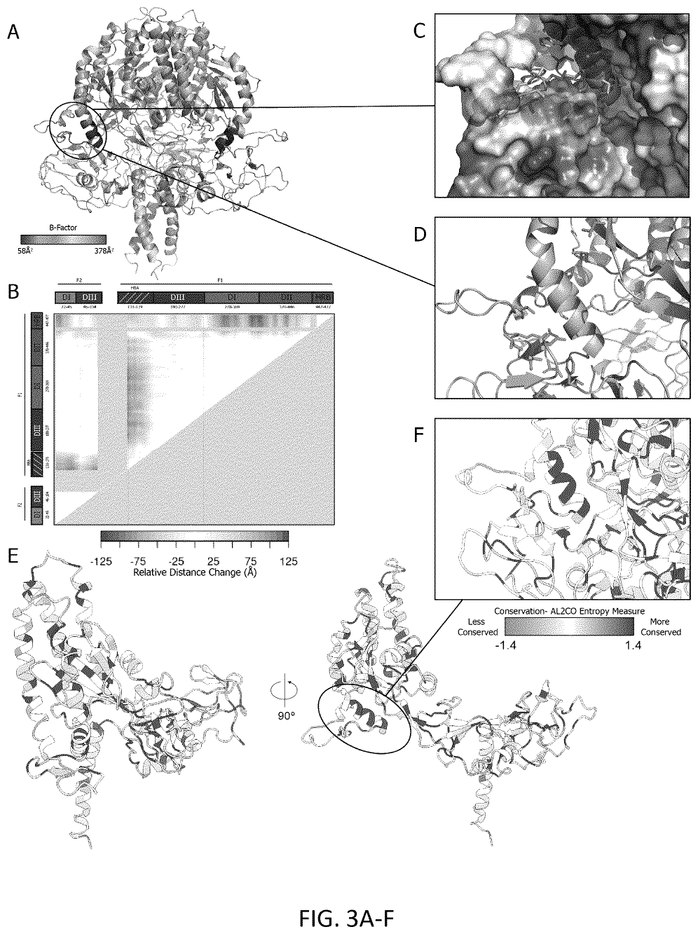

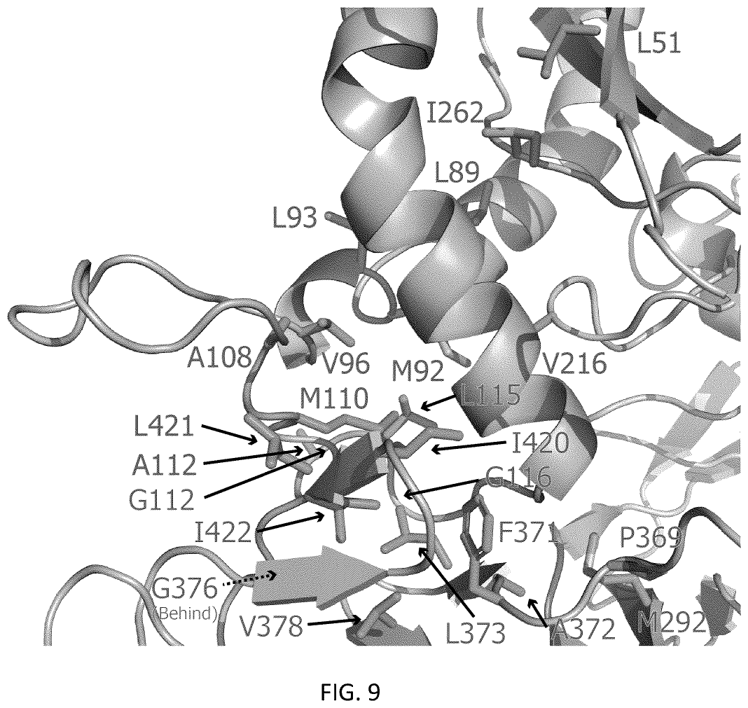

Insertion of the hydrophobic fusion peptide (FP) into the host membrane is an important step that leads to the fusion of the virus and host cell membranes. In the pre-fusion Lay V-F, a substantial portion of the FP is buried within a pocket formed between two Lay V-F protomers, requiring the FP to be released from this pocket during the pre-to-post fusion conformational transition (FIG. 3A-3F). Hydrophobic residues lining the interprotomer pocket into which the FP is buried in the pre-fusion LayV-F include L51, L89, M92, L93, V96, V216, and I262 from DIII, A108, M110, G112, A114, L115, and G116 from the N-terminal end of the FP upstream of the helical segment from the same protomer, and residues from the adjacent protomer include M292 from DI and P369, F371, A372, L373, G376, V378, 1420, L421, and 1422 from DII (FIG. 3D, FIG. 9).

Consistent with the close packing of the FP within an interprotomer pocket in the pre-fusion F conformation, the modeled coordinates exhibit the lowest B-factors at the FP, while HRB, its DII linker, and surrounding DI region have the highest B-factors in the structure (FIG. 3A). The HRA, HRB and FP undergo large shifts in their positions during pre- to post-fusion conformational change of the LayV-F protein (FIG. 3B). Although the FP is not resolved in the post-fusion state, based on its location it is expected to show a position shift similar to that of HRA. The burial of the pre-fusion FP and its interactions with the residues lining the interprotomer pocket indicate that the purpose of this pocket is to hold the FP in place to prevent premature conversion to the post-fusion form and therefore is the source of metastability of the pre-fusion LayV-F structure. Consistent with a key role in pre- to post-fusion conformational dynamics, the FP and the surrounding pocket are among the most conserved regions in Henipavirus fusion proteins (FIGS. 3E-3F). Release of the FP from this pocket occurs during conformation transition of the F protein from the pre-fusion to post-fusion conformation and disruption of this pocket can be the mechanism by which the FP is released to undergo these changes.

### Example 4

### Structural Conservation and Antigenic Diversity of Henipavirus Fusion Proteins.

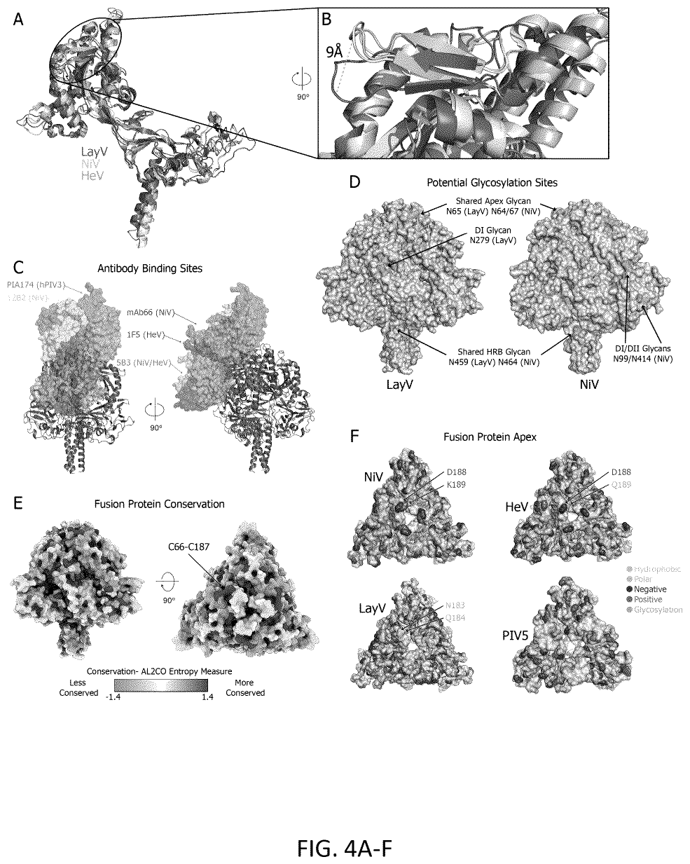

Despite low overall sequence conservation, the structural similarity of paramyxovirus fusion proteins is striking. Structures have previously been determined for the F proteins of two Henipaviruses in the pre-fusion state, NiV—F and HeV-F (2, 3). Despite having only 38% sequence identity with these viruses, a superimposition of LayV-F with these structures reveals a conserved architecture (FIG. 4A), with Cα RMSDs of 2.64 Å and 3.00 Å for LayV-F to NiV-F and HeV-F, respectively, with NiV-F and HeV-F differing between each other by 0.94 Å. One of the few areas between these species where there is a notable shift is in HRA. In the pre-fusion conformation, the central helix in HRA is broken by a short, two-strand beta sheet with a loop that extends to the exterior of the protein. As compared to the homologous HRA loop in NiV/HeV-F, in LayV-F there is a shift of ˜9 Å in the direction of the fusion peptide (FIG. 4B). It is unclear if this shift results in a mechanistic effect on the HRA region, although the loop resides within a site targeted by antibodies and shift in its position may alter antigenicity of the F protein. (FIG. 4C).

An overlay of all experimentally determined structures of paramyxovirus-targeted antibodies highlights the antigenicity of the HRA loop site and its adjacent regions (FIG. 4C). Several antibodies target DIII in the pre-fusion F near the HRA region and likely interfere with pre- to post-fusion conversion. In addition to the HRA loop shift described above, LayV-F and other paramyxovirus species show differences in their surface-exposed residues in these regions that could define the unique antigenic properties of the different F proteins. These include a glycosylation site in LayV-F at N279 (FIG. 4D), which is positioned between DI and DIII near several of the previously identified epitopes, specifically 15F, an HeV-neutralizing antibody and 5B3, a cross-reactive NiV/HeV-neutralizing antibody (6).

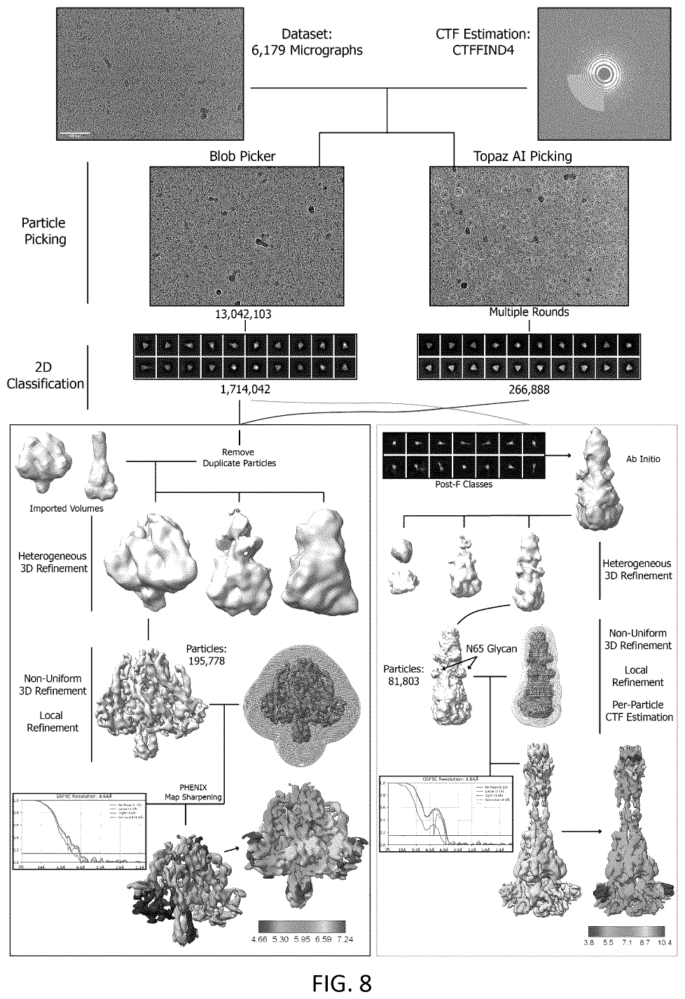

The sequences of both NiV-F and HeV-F have two plausible glycosylation sites at N64 and N67, both at the apex, though previous studies have established that only N67 is glycosylated (45, 46). The N65 glycosylation position in LayV-F is shifted by 8.2 Å away from the apex residues of HRA. This glycan was particularly visible in the cryo-EM reconstructions of the post-fusion conformation before local refinement, where the density at roughly the half-height point of the map corresponds to N65 (FIG. 8). Sequence alignment reveals that the LayV-F N65 glycosylation site is not homologous to either N64 or N67 of NiV/HeV, but rather is equivalent to residue 70 (FIG. 6). Viewing the fusion protein colored by sequence conservation further highlights the variability of this region (FIG. 4E). While there are highly conserved residues at discrete points of the surface, namely the highly conserved disulfide bond near the apex, the surrounding region ranges from moderately conserved to highly variable, especially in surface-accessible regions of DIII and the HRA loop. This variability includes differing residue types between LayV and other Henipaviruses, particularly at the apex site. The differences in the pre-fusion F protein trimer apex are highlighted by a negatively charged residue, D188, in both NiV and HeV, while the LayV-F protein instead has polar residues (N183, Q184) and an overall greater hydrophobic character. This residue arrangement in the pre-fusion Lay V-F trimer apex is more similar to that of the PIV5 pre-fusion F protein (FIG. 4F). This change in residue type is likely a source of antigenic variability between paramyxovirus F proteins. Consistent with this variability, antibody 12B2, that binds an epitope in pre-fusion NiV-F that includes the trimer apex (6, 11), did not show binding to LayV-F (FIG. 7F). Therefore, despite similar F protein architecture, antigenic variability within paramyxoviruses is driven by variability of surface residues.

### Example 5

Identification of Amino Acid Mutations that Stabilize the Pre-Fusion Protein Conformation.

The present invention provides designed mutations that are predicted to stabilize the pre-fusion conformation. Such stabilized pre-fusion F protein ectodomains have utility as immunogens for vaccine development against Paramyxoviruses/Henipaviruses, which include pathogens such as Nipah and Hendra. While these designs are based on the Langya virus F protein structure, these changes are translatable to the entire family of Paramyxoviruses/Henipaviruses.

A) stabilizing a kinked conformation by mutation of Glutamic Acid 134 to Proline.

B) stabilizing the DII domain by mutation of alanine 340 to cysteine and/or valine 364 to cysteine.

C) making one or more of the following mutations: glycine 99 to cysteine, isoleucine 109 to cysteine, isoleucine 167 to phenylalanine and serine 186 to proline.

D) stabilizing the DII domain by mutation of alanine 111 to cysteine and serine 423 to cysteine.

E) inhibition of HRA helix region to straighten its conformation by mutation of asparagine 150 to proline.

F) stabilization of prefusion HRA domain conformation by mutation of charged amino acids to non-charged amino acids lysine 55 to isoleucine, aspartic acid 168 to alanine, and arginine 171 to isoleucine.

G) stabilizing the HRB domain by mutation of serine 356 to cysteine and lysine 440 to cysteine.

H) a combination of the mutations of A) and F).

I) the combination of mutations of B) and G).

### Example 6

### Comparison of the Lay V-F Fusion Peptide (FP) Arrangement to Other Viral Fusion Peptides Highlights the Unique Nature of the Paramyxovirus FP.

Though the structure of LayV-F is strikingly similar to other known Henipavirus F structures, key differences in surface characteristics at the apex have implications for pan-Henipavirus or pan-paramyxovirus vaccine development. When developing vaccinations for a virus that is quickly mutating, or with many distinct strains, as observed with Henipaviruses, one can target relatively invariable sites for immunogen design. The pre-fusion LayV-F protein structure of the present invention helps to demonstrate the particularly variable nature of the apex and DIII in Henipavirus F proteins, a trait to be considered when selecting antigenic sites to target for a broad immunogenic response.

In contrast to existing structures of paramyxovirus fusion proteins that were determined in either pre- or post-fusion conformation, the present study resolves the LayV-F pre- and post-fusion structures within the same cryo-EM dataset, showing that for LayV-F, being in solution at ambient temperatures can allow the proteins to sample enough conformational states such that some will lead to conversion. Though paramyxovirus fusion proteins must be cleaved to enable fusion peptide insertion, prior studies have observed uncleaved fusion proteins assuming a post-fusion conformation (47).

The present inventive structures reveal a highly stable configuration of the fusion peptide (FP) in the pre-fusion F conformation, buried within an interprotomer pocket, which must be disrupted to transition to the post-fusion conformation. These observations provide evidence that FP configuration is a source of metastability in the pre-fusion LayV-F protein, where a shift in the position of the FP or the residues surrounding it in the interprotomer pocket too far from its pre-fusion configuration can lead to irreversible conversion to the post-fusion form. Without wishing to be bound by any particular theory, this together with the biochemical properties of the FP burial pocket, indicates tight control of FP positioning is an important part of pre-fusion metastability.

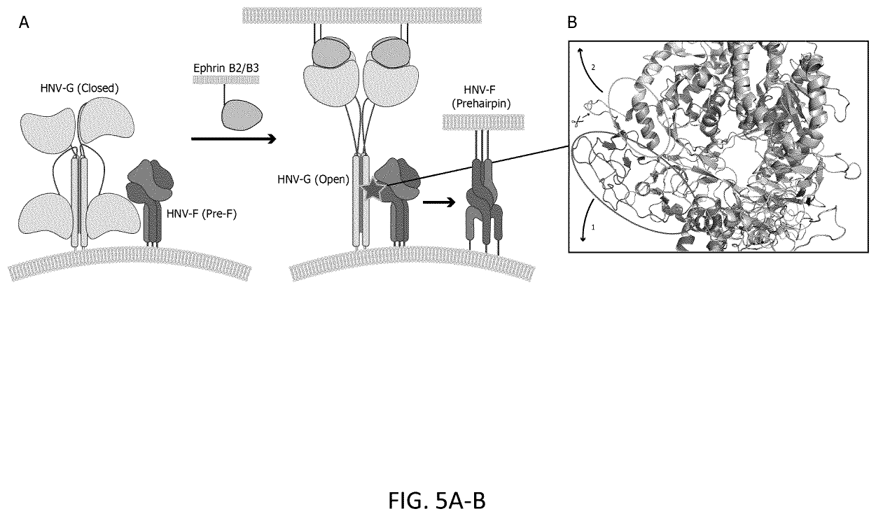

A question regarding paramyxovirus fusion is how the fusion process is first initiated. For the many virus families that utilize a fusion protein, the metastable pre-fusion state is maintained prior to a triggering event that initiates conformational changes. In many cases, this process involves cleavage and removal of entire domains of the fusion protein, such as with the removal of the S1 subunit from the SARS-COV-2 spike protein prior to fusion. The paramyxovirus fusion proteins, however, have no such attachment subunit to remove. Instead, paramyxoviruses utilize a separate attachment protein, which is thought to pass on a triggering signal to the fusion protein after receptor binding (33, 34) (FIG. 5A). While not wishing to be bound by any particular theory, within the Henipavirus and Morbilivirus genera, the attachment and fusion proteins are believed to form complexes prior to receptor engagement (33) (FIG. 5A). While this can indicate a mechanism where the attachment protein functions as a clamp on the fusion protein, maintaining the pre-fusion state before receptor binding, studies assaying fusion competency have demonstrated that the fusion protein alone is not sufficient for virus and host cell fusion. Without being bound by any particular theory, this can indicate that fusion triggering involves a more complex mechanism of interaction between the two proteins (9, 48).

The fusion peptide in the pre-fusion LayV-F protein is in a configuration that is conserved among paramyxoviruses and distinct from other class-I fusion proteins (FIG. 9). It is not yet known which residues are involved in the interaction between the attachment and fusion proteins. But, without wishing to be bound by theory, the role of the FP pocket created at the DII-DIII interface indicates that DII can be the site of the interaction. The triggering signal can be transferred by way of the attachment protein repositioning DII such that the fusion peptide is released from the pocket, irreversibly extending towards the apex (FIG. 5B). This mechanism can be consistent with previous paramyxovirus studies that suggested an attachment protein-binding role for DII (49, 50). Further support for this is the likelihood that the FP in the pre-fusion conformation is in a high-energy state due to the kink in the helix comprised of the FP from T119 through HRA up to N148. Such a helix shape can be brought about by the presence of a proline at the kink, introducing bond-angle limitations that force this shape into the helix (51). However, in the helix containing the FP, no proline is present. Without being bound by any particular theory, the bent shape can be induced by the FP being buried within and locked into the hydrophobic FP pocket. When disrupted, the position of the FP could move rapidly as the tension in the helix is resolved. Our study, has established that cleavage is not required for conformational conversion in paramyxovirus fusion proteins. Without being bound by any particular theory, existence of a high-energy state FP helix, held in place by an interprotomer DII, which is then disrupted by the action of an attachment protein can explain how Henipavirus attachment proteins activate their conjugate fusion proteins, and establishes the basis for how paramyxovirus fusion proteins maintain their metastability in the pre-fusion state.

### Example 7

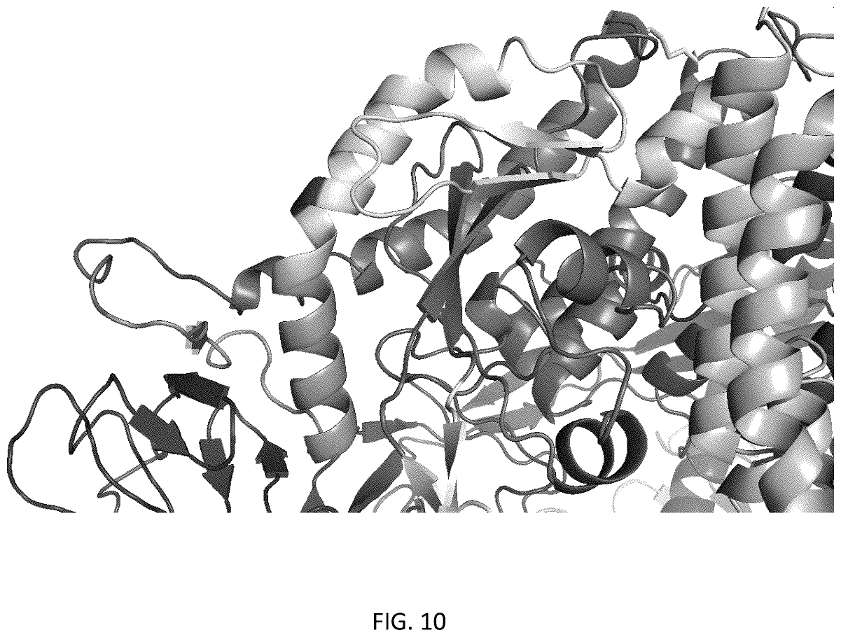

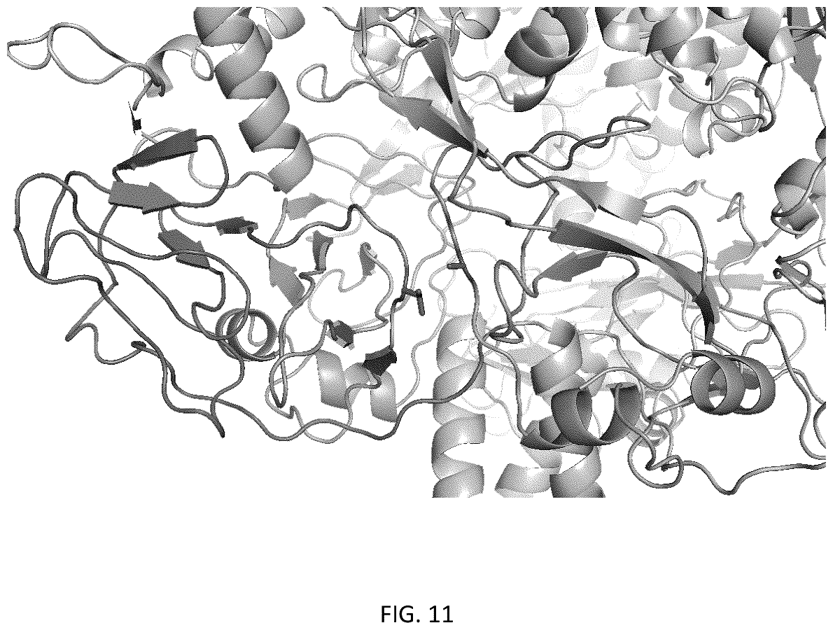

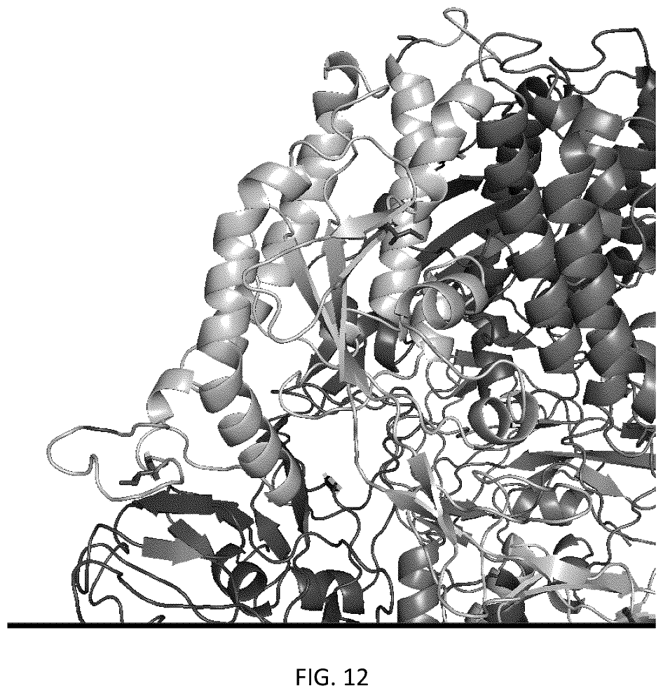

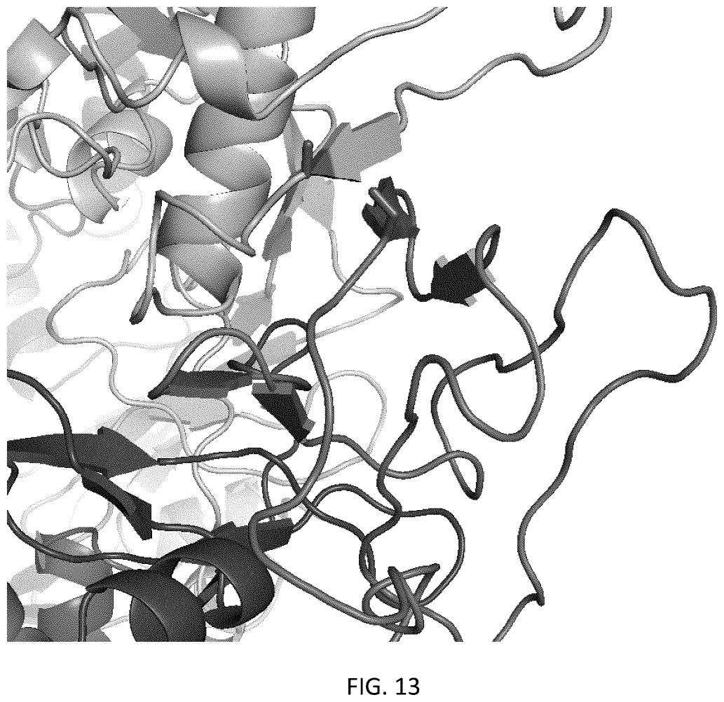

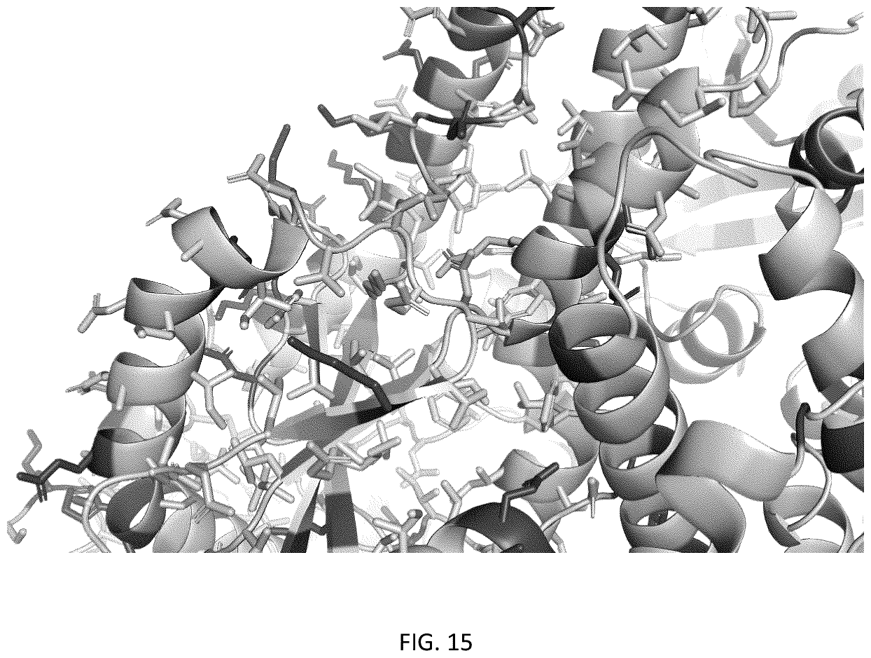

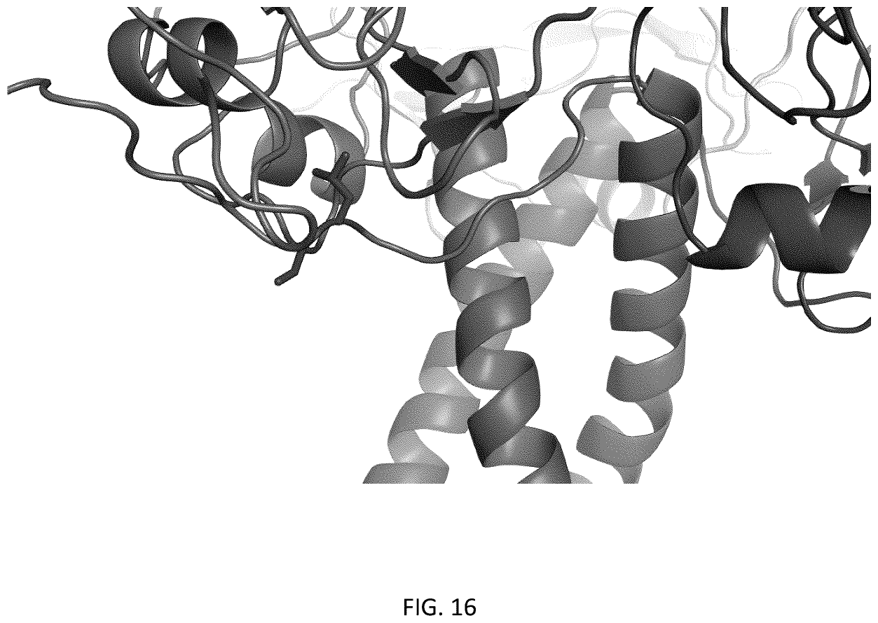

FIGS. 10-16 illustrate example structures of LayV-F proteins containing various mutations.

### Example 8

Below are amino acid sequences of some embodiments of fusion protein mutations disclosed herein, for Langya virus, Nipah virus and Hendra virus. Amino acid substitutions, as compared to wild-type sequences (see SEQ ID NOs: 1-3) are shown as underlined, bolded and highlighted text.

### Langya Virus (LayV) Sequences:

E134P mutation (SEQ ID NO: 4):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNPNAQAIANMKNAIQNTNEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
SKCAREKVVSSYVPRFALSDGLVYANCLNTICRCMDTDTPISQSLGTTVSLLDNKKCLV
YQVGDILISVGSYLGEGEYSADNVELGPPVVIDKIDIGNQLAGINQTLQNAEDYIEKSEEF
LKGINPSI

SEQ ID NO: 36, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACCCTAACGCCCAGGCCATTGCCAACATGAAGAACGCCATCCAGAAC
ACCAACGAGGCCGTGAAGCAGCTCCAGCTGGCCAACAAACAGACCCTGGCCGTGAT
CGACACCATCCGGGGAGAGATCAACAACAACATCATCCCCGTGATCAACCAGCTGA
GCTGTGACACCATCGGCCTGAGCGTGGGAATCAAGCTGACCCAGTACTACAGCGAG
ATCCTGACCGCCTTCGGACCCGCTCTGCAGAACCCTGTGAACACCAGAATCACCATC
CAGGCCATCAGCAGCGTGTTCAACCGGAACTTCGACGAGCTGCTGAAGATCATGGG
CTACACCAGCGGCGACCTGTATGAGATCCTGCACAGCGGCCTGATCCGGGGCAATA
TCATCGACGTGGACGTGGAAGCCGGCTATATCGCCCTGGAAATCGAGTTCCCCAACC
TGACACTGGTGCCCAACGCCGTGGTGCAAGAGCTGATGCCCATCTCCTACAACGTGG
ACGGCGACGAGTGGGTTACCCTGGTGCCTAGATTCGTGCTGACCCGGACAACCCTGC
TGAGCAACATCGATACCAGCAGATGCACCGTGACCGAGAGCAGCGTGATCTGCGAC
AACGATTACGCCCTGCCTATGAGCTACGAGCTGATCGGATGTCTGCAGGGCGACAC
CAGCAAGTGCGCCAGAGAAAAGGTGGTGTCCAGCTACGTGCCCAGATTCGCCCTGT
CTGACGGACTGGTGTACGCCAACTGCCTGAACACCATCTGCCGGTGCATGGACACC
GACACACCTATCTCTCAGAGCCTGGGCACCACCGTGTCTCTGCTGGATAACAAGAAA
TGCCTGGTGTACCAAGTGGGCGACATCCTGATCTCCGTGGGCTCTTATCTCGGCGAG
GGCGAGTACAGCGCCGACAATGTTGAACTGGGCCCTCCTGTGGTCATCGACAAGAT
CGACATCGGCAATCAGCTGGCCGGCATCAATCAGACCCTGCAGAACGCCGAGGACT
ACATCGAGAAGTCTGAAGAGTTCCTGAAGGGCATCAACCCCAGCATCGGATCCGGA
TACATCCCCGAGGCCCCCAGAGATGGCCAGGCCTACGTGCGGAAGGACGGCGAGTG
GGTACTGCTGAGCACATTCCTGGGCAGATCCCTGGAGGTGCTGTTCCAGGGCCCAGG
CCATCACCACCATCACCACCATCATAGCGCCTGGTCCCACCCCCAGTTCGAGAAGGG
CGGCGGTAGTGGAGGGGGCGGATCTGGCGGCTCAGCTTGGAGCCACCCCCAGTTCG
AAAAGTGATAATGACTCGAG
A340C and V364C mutations (SEQ ID NO: 5):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKNAIQNTNEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYCLPMSYELIGCLQGDT
SKCAREKVCSSYVPRFALSDGLVYANCLNTICRCMDTDTPISQSLGTTVSLLDNKKCLV
YQVGDILISVGSYLGEGEYSADNVELGPPVVIDKIDIGNQLAGINQTLQNAEDYIEKSEEF
LKGINPSI

SEQ ID NO: 37, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGA
TCGACACCATCCGGGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACTGCCTGCCTATGAGCTACGAGCTGATCGGATGTCTGCAGGGCGAC
ACCAGCAAGTGCGCCAGAGAAAAGGTGTGCTCCAGCTACGTGCCCAGATTCGCCCT
GTCTGACGGACTGGTGTACGCCAACTGCCTGAACACCATCTGCCGGTGCATGGACAC
CGACACACCTATCTCTCAGAGCCTGGGCACCACCGTGTCTCTGCTGGATAACAAGAA
ATGCCTGGTGTACCAAGTGGGCGACATCCTGATCTCCGTGGGCTCTTATCTCGGCGA
GGGCGAGTACAGCGCCGACAATGTTGAACTGGGCCCTCCTGTGGTCATCGACAAGA
TCGACATCGGCAATCAGCTGGCCGGCATCAATCAGACCCTGCAGAACGCCGAGGAC
TACATCGAGAAGTCTGAAGAGTTCCTGAAGGGCATCAACCCCAGCATCGGATCCGG
ATACATCCCCGAGGCCCCCAGAGATGGCCAGGCCTACGTGCGGAAGGACGGCGAGT
GGGTACTGCTGAGCACATTCCTGGGCAGATCCCTGGAGGTGCTGTTCCAGGGCCCAG
GCCATCACCACCATCACCACCATCATAGCGCCTGGTCCCACCCCCAGTTCGAGAAGG
GCGGCGGTAGTGGAGGGGGCGGATCTGGCGGCTCAGCTTGGAGCCACCCCCAGTTC
GAAAAGTGATAATGACTCGAG
G99C, I109C, I167F and S186P mutations (SEQ ID NO: 6):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSCNNKYRFAGACMA
GVALGVATAATVTAGIALHRSNENAQAIANMKNAIQNTNEAVKQLQLANKQTLAVFD
TIRGEINNNIIPVINQLPCDTIGLSVGIKLTQYYSEILTAFGPALQNPVNTRITIQAISSVENR
NFDELLKIMGYTSGDLYEILHSGLIRGNIIDVDVEAGYIALEIEFPNLTLVPNAVVQELMPI
SYNVDGDEWVTLVPRFVLTRTTLLSNIDTSRCTVTESSVICDNDYALPMSYELIGCLQGD
TSKCAREKVVSSYVPRFALSDGLVYANCLNTICRCMDTDTPISQSLGTTVSLLDNKKCL
VYQVGDILISVGSYLGEGEYSADNVELGPPVVIDKIDIGNQLAGINQTLQNAEDYIEKSEE
FLKGINPSI

SEQ ID NO: 38, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCTGCAACAACAAGTACAGATTCGCTGGCGCCTGCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGT
TCGACACCATCCGGGGAGAGATCAACAACAACATCATCCCCGTGATCAACCAGCTG
CCCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCAGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTCCGTGGGCTCTTATCTCGGCG
AGGGCGAGTACAGCGCCGACAATGTTGAACTGGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
A11C and S423C mutations (SEQ ID NO: 7):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMC
GVALGVATAATVTAGIALHRSNENAQAIANMKNAIQNTNEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
SKCAREKVVSSYVPRFALSDGLVYANCLNTICRCMDTDTPISQSLGTTVSLLDNKKCLV
YQVGDILICVGSYLGEGEYSADNVELGPPVVIDKIDIGNQLAGINQTLQNAEDYIEKSEEF
LKGINPSI

SEQ ID NO: 39, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGT
GCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGA
TCGACACCATCCGGGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCAGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTGCGTGGGCTCTTATCTCGGCG
AGGGCGAGTACAGCGCCGACAATGTTGAACTGGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
N150P mutation (SEQ ID NO: 8):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKNAIQNTPEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
SKCAREKVVSSYVPRFALSDGLVYANCLNTICRCMDTDTPISQSLGTTVSLLDNKKCLV
YQVGDILISVGSYLGEGEYSADNVELGPPVVIDKIDIGNQLAGINQTLQNAEDYIEKSEEF
LKGINPSI

SEQ ID NO: 40, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCCCCGAGGCCGTGAAGCAGCTCCAGCTGGCCAACAAACAGACCCTGGCCGTGA
TCGACACCATCCGGGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCAGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTCCGTGGGCTCTTATCTCGGCG
AGGGCGAGTACAGCGCCGACAATGTTGAACTGGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
K55I, D168A and R171I mutations (SEQ ID NO: 9):
MAFLKSAIICYLLFYPHIVKSSLHYDSLSKVGIIKGLTYNYKIKGSPSTKLMVV
ILIPNIDGVRNCTQKQFDEYKNLVKNVLEPVKLALNAMLDNVKSGNNKYRFAGAIMAG
VALGVATAATVTAGIALHRSNENAQAIANMKNAIQNTNEAVKQLQLANKQTLAVIATII
GEINNNIIPVINQLSCDTIGLSVGIKLTQYYSEILTAFGPALQNPVNTRITIQAISSVFNRNFD
ELLKIMGYTSGDLYEILHSGLIRGNIIDVDVEAGYIALEIEFPNLTLVPNAVVQELMPISYN
VDGDEWVTLVPRFVLTRTTLLSNIDTSRCTVTESSVICDNDYALPMSYELIGCLQGDTSK
CAREKVVSSYVPRFALSDGLVYANCLNTICRCMDTDTPISQSLGTTVSLLDNKKCLVYQ
VGDILISVGSYLGEGEYSADNVELGPPVVIDKIDIGNQLAGINQTLQNAEDYIEKSEEFLK
GINPSI

SEQ ID NO: 41, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
TCCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGA
TCGCCACCATCATCGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCAGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTCCGTGGGCTCTTATCTCGGCG
AGGGCGAGTACAGCGCCGACAATGTTGAACTGGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
S365C and L440C mutations (SEQ ID NO: 10):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKNAIQNTNEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
CKCAREKVVSSYVPRFALSDGLVYANCLNTICRCMDTDTPISQSLGTTVSLLDNKKCLV
YQVGDILISVGSYLGEGEYSADNVECGPPVVIDKIDIGNQLAGINQTLQNAEDYIEKSEEF
LKGINPSI

SEQ ID NO: 42, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGA
TCGACACCATCCGGGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCTGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTCCGTGGGCTCTTATCTCGGCG
AGGGCGAGTACAGCGCCGACAATGTTGAATGCGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
K55I, E134P, D168A and R171I mutations (SEQ ID NO: 11);
MAFLKSAIICYLLFYPHIVKSSLHYDSLSKVGIIKGLTYNYKIKGSPSTKLMVV
ILIPNIDGVRNCTQKQFDEYKNLVKNVLEPVKLALNAMLDNVKSGNNKYRFAGAIMAG
VALGVATAATVTAGIALHRSNPNAQAIANMKNAIQNTNEAVKQLQLANKQTLAVIATII
GEINNNIIPVINQLSCDTIGLSVGIKLTQYYSEILTAFGPALQNPVNTRITIQAISSVENRNFD
ELLKIMGYTSGDLYEILHSGLIRGNIIDVDVEAGYIALEIEFPNLTLVPNAVVQELMPISYN
VDGDEWVTLVPRFVLTRTTLLSNIDTSRCTVTESSVICDNDYALPMSYELIGCLQGDTSK
CAREKVVSSYVPRFALSDGLVYANCLNTICRCMDTDTPISQSLGTTVSLLDNKKCLVYQ
VGDILISVGSYLGEGEYSADNVELGPPVVIDKIDIGNQLAGINQTLQNAEDYIEKSEEFLK
GINPSI

SEQ ID NO:43, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
TCCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACCCTAACGCCCAGGCCATTGCCAACATGAAGAACGCCATCCAGAAC
ACCAACGAGGCCGTGAAGCAGCTCCAGCTGGCCAACAAACAGACCCTGGCCGTGAT
CGCCACCATCATCGGAGAGATCAACAACAACATCATCCCCGTGATCAACCAGCTGA
GCTGTGACACCATCGGCCTGAGCGTGGGAATCAAGCTGACCCAGTACTACAGCGAG
ATCCTGACCGCCTTCGGACCCGCTCTGCAGAACCCTGTGAACACCAGAATCACCATC
CAGGCCATCAGCAGCGTGTTCAACCGGAACTTCGACGAGCTGCTGAAGATCATGGG
CTACACCAGCGGCGACCTGTATGAGATCCTGCACAGCGGCCTGATCCGGGGCAATA
TCATCGACGTGGACGTGGAAGCCGGCTATATCGCCCTGGAAATCGAGTTCCCCAACC
TGACACTGGTGCCCAACGCCGTGGTGCAAGAGCTGATGCCCATCTCCTACAACGTGG
ACGGCGACGAGTGGGTTACCCTGGTGCCTAGATTCGTGCTGACCCGGACAACCCTGC
TGAGCAACATCGATACCAGCAGATGCACCGTGACCGAGAGCAGCGTGATCTGCGAC
AACGATTACGCCCTGCCTATGAGCTACGAGCTGATCGGATGTCTGCAGGGCGACAC
CAGCAAGTGCGCCAGAGAAAAGGTGGTGTCCAGCTACGTGCCCAGATTCGCCCTGT
CTGACGGACTGGTGTACGCCAACTGCCTGAACACCATCTGCCGGTGCATGGACACC
GACACACCTATCTCTCAGAGCCTGGGCACCACCGTGTCTCTGCTGGATAACAAGAAA
TGCCTGGTGTACCAAGTGGGCGACATCCTGATCTCCGTGGGCTCTTATCTCGGCGAG
GGCGAGTACAGCGCCGACAATGTTGAACTGGGCCCTCCTGTGGTCATCGACAAGAT
CGACATCGGCAATCAGCTGGCCGGCATCAATCAGACCCTGCAGAACGCCGAGGACT
ACATCGAGAAGTCTGAAGAGTTCCTGAAGGGCATCAACCCCAGCATCGGATCCGGA
TACATCCCCGAGGCCCCCAGAGATGGCCAGGCCTACGTGCGGAAGGACGGCGAGTG
GGTACTGCTGAGCACATTCCTGGGCAGATCCCTGGAGGTGCTGTTCCAGGGCCCAGG
CCATCACCACCATCACCACCATCATAGCGCCTGGTCCCACCCCCAGTTCGAGAAGGG
CGGCGGTAGTGGAGGGGGCGGATCTGGCGGCTCAGCTTGGAGCCACCCCCAGTTCG
AAAAGTGATAATGACTCGAG
K55I, N150P, D168A and R171I mutations (SEQ ID NO: 12);
MAFLKSAIICYLLFYPHIVKSSLHYDSLSKVGIIKGLTYNYKIKGSPSTKLMVV
ILIPNIDGVRNCTQKQFDEYKNLVKNVLEPVKLALNAMLDNVKSGNNKYRFAGAIMAG
VALGVATAATVTAGIALHRSNENAQAIANMKNAIQNTPEAVKQLQLANKQTLAVIATII
GEINNNIIPVINQLSCDTIGLSVGIKLTQYYSEILTAFGPALQNPVNTRITIQAISSVFNRNFD
ELLKIMGYTSGDLYEILHSGLIRGNIIDVDVEAGYIALEIEFPNLTLVPNAVVQELMPISYN
VDGDEWVTLVPRFVLTRTTLLSNIDTSRCTVTESSVICDNDYALPMSYELIGCLQGDTSK
CAREKVVSSYVPRFALSDGLVYANCLNTICRCMDTDTPISQSLGTTVSLLDNKKCLVYQ
VGDILISVGSYLGEGEYSADNVELGPPVVIDKIDIGNQLAGINQTLQNAEDYIEKSEEFLK
GINPSI

SEQ ID NO: 44, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
TCCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCCCCGAGGCCGTGAAGCAGCTCCAGCTGGCCAACAAACAGACCCTGGCCGTGA
TCGCCACCATCATCGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACGCCCTGCCTATGAGCTACGAGCTGATCGGATGTCTGCAGGGCGA
CACCAGCAAGTGCGCCAGAGAAAAGGTGGTGTCCAGCTACGTGCCCAGATTCGCCC
TGTCTGACGGACTGGTGTACGCCAACTGCCTGAACACCATCTGCCGGTGCATGGACA
CCGACACACCTATCTCTCAGAGCCTGGGCACCACCGTGTCTCTGCTGGATAACAAGA
AATGCCTGGTGTACCAAGTGGGCGACATCCTGATCTCCGTGGGCTCTTATCTCGGCG
AGGGCGAGTACAGCGCCGACAATGTTGAACTGGGCCCTCCTGTGGTCATCGACAAG
ATCGACATCGGCAATCAGCTGGCCGGCATCAATCAGACCCTGCAGAACGCCGAGGA
CTACATCGAGAAGTCTGAAGAGTTCCTGAAGGGCATCAACCCCAGCATCGGATCCG
GATACATCCCCGAGGCCCCCAGAGATGGCCAGGCCTACGTGCGGAAGGACGGCGAG
TGGGTACTGCTGAGCACATTCCTGGGCAGATCCCTGGAGGTGCTGTTCCAGGGCCCA
GGCCATCACCACCATCACCACCATCATAGCGCCTGGTCCCACCCCCAGTTCGAGAAG
GGCGGCGGTAGTGGAGGGGGCGGATCTGGCGGCTCAGCTTGGAGCCACCCCCAGTT
CGAAAAGTGATAATGACTCGAG
A340C, S356C, V364C and L440C mutations (SEQ ID NO: 13):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKNAIQNTNEAVKQLQLANKQTLAVIDT
IRGEINNNIIPVINQLSCDTIGLSVGIKLTQYYSEILTAFGPALQNPVNTRITIQAISSVENRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYCLPMSYELIGCLQGDT
CKCAREKVCSSYVPRFALSDGLVYANCLNTICRCMDTDTPISQSLGTTVSLLDNKKCLV
YQVGDILISVGSYLGEGEYSADNVECGPPVVIDKIDIGNQLAGINQTLQNAEDYIEKSEEF
LKGINPSI

SEQ ID NO: 45, below, is an example nucleotide sequence encoding the above amino acid sequence:

ATGGCCTTTCTGAAGTCCGCCATCATCTGCTACCTGCTGTTCTACCCTCAC
ATCGTGAAGTCCAGCCTGCACTACGACAGCCTGAGCAAAGTGGGCATCATCAAGGG
CCTGACCTACAACTACAAGATCAAGGGCAGCCCCAGCACCAAGCTGATGGTGGTCA
AGCTGATCCCCAACATCGACGGCGTGCGGAACTGTACCCAGAAGCAGTTCGACGAG
TACAAGAACCTGGTCAAGAACGTGCTGGAACCCGTGAAGCTGGCCCTGAACGCCAT
GCTGGACAATGTGAAGTCCGGCAACAACAAGTACAGATTCGCTGGCGCCATCATGG
CCGGCGTTGCACTTGGAGTTGCCACAGCCGCTACAGTGACAGCCGGAATTGCCCTGC
ACAGAAGCAACGAGAACGCCCAGGCCATTGCCAACATGAAGAACGCCATCCAGAA
CACCAACGAGGCCGTGAAGCAGCTCCAGCTGGCCAACAAACAGACCCTGGCCGTGA
TCGACACCATCCGGGGAGAGATCAACAACAACATCATCCCCGTGATCAACCAGCTG
AGCTGTGACACCATCGGCCTGAGCGTGGGAATCAAGCTGACCCAGTACTACAGCGA
GATCCTGACCGCCTTCGGACCCGCTCTGCAGAACCCTGTGAACACCAGAATCACCAT
CCAGGCCATCAGCAGCGTGTTCAACCGGAACTTCGACGAGCTGCTGAAGATCATGG
GCTACACCAGCGGCGACCTGTATGAGATCCTGCACAGCGGCCTGATCCGGGGCAAT
ATCATCGACGTGGACGTGGAAGCCGGCTATATCGCCCTGGAAATCGAGTTCCCCAA
CCTGACACTGGTGCCCAACGCCGTGGTGCAAGAGCTGATGCCCATCTCCTACAACGT
GGACGGCGACGAGTGGGTTACCCTGGTGCCTAGATTCGTGCTGACCCGGACAACCC
TGCTGAGCAACATCGATACCAGCAGATGCACCGTGACCGAGAGCAGCGTGATCTGC
GACAACGATTACTGCCTGCCTATGAGCTACGAGCTGATCGGATGTCTGCAGGGCGAC
ACCTGCAAGTGCGCCAGAGAAAAGGTGTGCTCCAGCTACGTGCCCAGATTCGCCCT
GTCTGACGGACTGGTGTACGCCAACTGCCTGAACACCATCTGCCGGTGCATGGACAC
CGACACACCTATCTCTCAGAGCCTGGGCACCACCGTGTCTCTGCTGGATAACAAGAA
ATGCCTGGTGTACCAAGTGGGCGACATCCTGATCTCCGTGGGCTCTTATCTCGGCGA
GGGCGAGTACAGCGCCGACAATGTTGAATGCGGCCCTCCTGTGGTCATCGACAAGA
TCGACATCGGCAATCAGCTGGCCGGCATCAATCAGACCCTGCAGAACGCCGAGGAC
TACATCGAGAAGTCTGAAGAGTTCCTGAAGGGCATCAACCCCAGCATCGGATCCGG
ATACATCCCCGAGGCCCCCAGAGATGGCCAGGCCTACGTGCGGAAGGACGGCGAGT
GGGTACTGCTGAGCACATTCCTGGGCAGATCCCTGGAGGTGCTGTTCCAGGGCCCAG
GCCATCACCACCATCACCACCATCATAGCGCCTGGTCCCACCCCCAGTTCGAGAAGG
GCGGCGGTAGTGGAGGGGGCGGATCTGGCGGCTCAGCTTGGAGCCACCCCCAGTTC
GAAAAGTGATAATGACTCGAG
Nipah virus (NiV) sequences:
K139P mutation (SEQ ID NO: 14):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMPNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL

SEQ ID NO: 46, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGCCTAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACGCCACCCCCATGACCAACAACATGAGGG
AGTGCCTGACCGGCAGCACCGAGAAGTGCCCCAGGGAGCTGGTGGTGAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCATCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
A345C and V369C mutations (SEQ ID NO: 15):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYCTPMTNNMRECLTGS
TEKCPRELVCSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL

SEQ ID NO: 47, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACTGCACCCCCATGACCAACAACATGAGGG
AGTGCCTGACCGGCAGCACCGAGAAGTGCCCCAGGGAGCTGGTGTGCAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCATCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
A116C and S428C mutations (SEQ ID NO: 16):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MCGVAIGIATAAQITAGVALYEAMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIICLGKYLGSVNYNSEGIAIGPPVFTDKVDISSQISSMNQSLQQSKDYIKEAQRL
LDTVNPSL

SEQ ID NO: 48, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGTGCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACGCCACCCCCATGACCAACAACATGAGGG
AGTGCCTGACCGGCAGCACCGAGAAGTGCCCCAGGGAGCTGGTGGTGAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCTGCCT
GGGCAAGTACCTGGGCAGCGTGAACTACAACAGCGAGGGCATCGCCATCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
N155P mutation (SEQ ID NO: 17):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKSSIESTPEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL

SEQ ID NO: 49, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCCCCGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACGCCACCCCCATGACCAACAACATGAGGG
AGTGCCTGACCGGCAGCACCGAGAAGTGCCCCAGGGAGCTGGTGGTGAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCATCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
K60I, T173A and Q176I mutations (SEQ ID NO: 18):
MVVILDKRCYCNLLILILMISECSVGILHYEKLSKIGLVKGVTRKYKIKSNPLT
KDIVIIMIPNVSNMSQCTGSVMENYKTRLNGILTPIKGALEIYKNNTHDLVGDVRLAGVI
MAGVAIGIATAAQITAGVALYEAMKNADNINKLKSSIESTNEAVVKLQETAEKTVYVLA
ALIDYINTNLVPTIDKISCKQTELSLDLALSKYLSDLLFVFGPNLQDPVSNSMTIQAISQAF
GGNYETLLRTLGYATEDFDDLLESDSITGQIIYVDLSSYYIIVRVYFPILTEIQQAYIQELLP
VSFNNDNSEWISIVPNFILVRNTLISNIEIGFCLITKRSVICNQDYATPMTNNMRECLTGST
EKCPRELVVSSHVPRFALSNGVLFANCISVTCQCQTTGRAISQSGEQTLLMIDNTTCPTA
VLGNVIISLGKYLGSVNYNSEGIAIGPPVFTDKVDISSQISSMNQSLQQSKDYIKEAQRLL
DTVNPSL

SEQ ID NO: 50, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCATCATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGGCCGCCCTGATCGACTACATCAACACCAACCTGGTGCC
CACCATCGACAAGATCAGCTGCAAGCAGACCGAGCTGAGCCTGGACCTGGCCCTGA
GCAAGTACCTGAGCGACCTGCTGTTCGTGTTCGGCCCCAACCTGCAGGACCCCGTGA
GCAACAGCATGACCATCCAGGCCATCAGCCAGGCCTTCGGCGGCAACTACGAGACC
CTGCTGAGGACCCTGGGCTACGCCACCGAGGACTTCGACGACCTGCTGGAGAGCGA
CAGCATCACCGGCCAGATCATCTACGTGGACCTGAGCAGCTACTACATCATCGTGAG
GGTGTACTTCCCCATCCTGACCGAGATCCAGCAGGCCTACATCCAGGAGCTGCTGCC
CGTGAGCTTCAACAACGACAACAGCGAGTGGATCAGCATCGTGCCCAACTTCATCCT
GGTGAGGAACACCCTGATCAGCAACATCGAGATCGGCTTCTGCCTGATCACCAAGA
GGAGCGTGATCTGCAACCAGGACTACGCCACCCCCATGACCAACAACATGAGGGAG
TGCCTGACCGGCAGCACCGAGAAGTGCCCCAGGGAGCTGGTGGTGAGCAGCCACGT
GCCCAGGTTCGCCCTGAGCAACGGCGTGCTGTTCGCCAACTGCATCAGCGTGACCTG
CCAGTGCCAGACCACCGGCAGGGCCATCAGCCAGAGCGGCGAGCAGACCCTGCTGA
TGATCGACAACACCACCTGCCCCACCGCCGTGCTGGGCAACGTGATCATCAGCCTGG
GCAAGTACCTGGGCAGCGTGAACTACAACAGCGAGGGCATCGCCATCGGCCCCCCC
GTGTTCACCGACAAGGTGGACATCAGCAGCCAGATCAGCAGCATGAACCAGAGCCT
GCAGCAGAGCAAGGACTACATCAAGGAGGCCCAGAGGCTGCTGGACACCGTGAAC
CCCAGCCTG
E361C and I445C mutations (SEQ ID NO: 19):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TECKCPRELVVSSHVPRFALSNGVLFANCISVTCQCQTTGRAISQSGEQTLLMIDNTTCP
TAVLGNVIISLGKYLGSVNYNSEGIACGPPVFTDKVDISSQISSMNQSLQQSKDYIKEAQR
LLDTVNPSL

SEQ ID NO: 51, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACGCCACCCCCATGACCAACAACATGAGGG
AGTGCCTGACCGGCAGCACCTGCAAGTGCCCCAGGGAGCTGGTGGTGAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCTGCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
K60I, K139P, T173A and Q1761 mutations (SEQ ID NO: 20):
MVVILDKRCYCNLLILILMISECSVGILHYEKLSKIGLVKGVTRKYKIKSNPLT
KDIVIIMIPNVSNMSQCTGSVMENYKTRLNGILTPIKGALEIYKNNTHDLVGDVRLAGVI
MAGVAIGIATAAQITAGVALYEAMPNADNINKLKSSIESTNEAVVKLQETAEKTVYVLA
ALIDYINTNLVPTIDKISCKQTELSLDLALSKYLSDLLFVFGPNLQDPVSNSMTIQAISQAF
GGNYETLLRTLGYATEDFDDLLESDSITGQIIYVDLSSYYIIVRVYFPILTEIQQAYIQELLP
VSFNNDNSEWISIVPNFILVRNTLISNIEIGFCLITKRSVICNQDYATPMTNNMRECLTGST
EKCPRELVVSSHVPRFALSNGVLFANCISVTCQCQTTGRAISQSGEQTLLMIDNTTCPTA
VLGNVIISLGKYLGSVNYNSEGIAIGPPVFTDKVDISSQISSMNQSLQQSKDYIKEAQRLL
DTVNPSL

SEQ ID NO: 52, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCATCATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGCCTAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGGCCGCCCTGATCGACTACATCAACACCAACCTGGTGCC
CACCATCGACAAGATCAGCTGCAAGCAGACCGAGCTGAGCCTGGACCTGGCCCTGA
GCAAGTACCTGAGCGACCTGCTGTTCGTGTTCGGCCCCAACCTGCAGGACCCCGTGA
GCAACAGCATGACCATCCAGGCCATCAGCCAGGCCTTCGGCGGCAACTACGAGACC
CTGCTGAGGACCCTGGGCTACGCCACCGAGGACTTCGACGACCTGCTGGAGAGCGA
CAGCATCACCGGCCAGATCATCTACGTGGACCTGAGCAGCTACTACATCATCGTGAG
GGTGTACTTCCCCATCCTGACCGAGATCCAGCAGGCCTACATCCAGGAGCTGCTGCC
CGTGAGCTTCAACAACGACAACAGCGAGTGGATCAGCATCGTGCCCAACTTCATCCT
GGTGAGGAACACCCTGATCAGCAACATCGAGATCGGCTTCTGCCTGATCACCAAGA
GGAGCGTGATCTGCAACCAGGACTACGCCACCCCCATGACCAACAACATGAGGGAG
TGCCTGACCGGCAGCACCGAGAAGTGCCCCAGGGAGCTGGTGGTGAGCAGCCACGT
GCCCAGGTTCGCCCTGAGCAACGGCGTGCTGTTCGCCAACTGCATCAGCGTGACCTG
CCAGTGCCAGACCACCGGCAGGGCCATCAGCCAGAGCGGCGAGCAGACCCTGCTGA
TGATCGACAACACCACCTGCCCCACCGCCGTGCTGGGCAACGTGATCATCAGCCTGG
GCAAGTACCTGGGCAGCGTGAACTACAACAGCGAGGGCATCGCCATCGGCCCCCCC
GTGTTCACCGACAAGGTGGACATCAGCAGCCAGATCAGCAGCATGAACCAGAGCCT
GCAGCAGAGCAAGGACTACATCAAGGAGGCCCAGAGGCTGCTGGACACCGTGAAC
CCCAGCCTG
K601, N155P, T173A and Q176I mutations (SEQ ID NO: 21):
MVVILDKRCYCNLLILILMISECSVGILHYEKLSKIGLVKGVTRKYKIKSNPLT
KDIVIIMIPNVSNMSQCTGSVMENYKTRLNGILTPIKGALEIYKNNTHDLVGDVRLAGVI
MAGVAIGIATAAQITAGVALYEAMKNADNINKLKSSIESTPEAVVKLQETAEKTVYVLA
ALIDYINTNLVPTIDKISCKQTELSLDLALSKYLSDLLFVFGPNLQDPVSNSMTIQAISQAF
GGNYETLLRTLGYATEDFDDLLESDSITGQIIYVDLSSYYIIVRVYFPILTEIQQAYIQELLP
VSFNNDNSEWISIVPNFILVRNTLISNIEIGFCLITKRSVICNQDYATPMTNNMRECLTGST
EKCPRELVVSSHVPRFALSNGVLFANCISVTCQCQTTGRAISQSGEQTLLMIDNTTCPTA
VLGNVIISLGKYLGSVNYNSEGIAIGPPVFTDKVDISSQISSMNQSLQQSKDYIKEAQRLL
DTVNPSL

SEQ ID NO: 53, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCATCATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCCCCGAGGCCGTGGTGAAGCTGCAGGAGACCGCCGA
GAAGACCGTGTACGTGCTGGCCGCCCTGATCGACTACATCAACACCAACCTGGTGCC
CACCATCGACAAGATCAGCTGCAAGCAGACCGAGCTGAGCCTGGACCTGGCCCTGA
GCAAGTACCTGAGCGACCTGCTGTTCGTGTTCGGCCCCAACCTGCAGGACCCCGTGA
GCAACAGCATGACCATCCAGGCCATCAGCCAGGCCTTCGGCGGCAACTACGAGACC
CTGCTGAGGACCCTGGGCTACGCCACCGAGGACTTCGACGACCTGCTGGAGAGCGA
CAGCATCACCGGCCAGATCATCTACGTGGACCTGAGCAGCTACTACATCATCGTGAG
GGTGTACTTCCCCATCCTGACCGAGATCCAGCAGGCCTACATCCAGGAGCTGCTGCC
CGTGAGCTTCAACAACGACAACAGCGAGTGGATCAGCATCGTGCCCAACTTCATCCT
GGTGAGGAACACCCTGATCAGCAACATCGAGATCGGCTTCTGCCTGATCACCAAGA
GGAGCGTGATCTGCAACCAGGACTACGCCACCCCCATGACCAACAACATGAGGGAG
TGCCTGACCGGCAGCACCGAGAAGTGCCCCAGGGAGCTGGTGGTGAGCAGCCACGT
GCCCAGGTTCGCCCTGAGCAACGGCGTGCTGTTCGCCAACTGCATCAGCGTGACCTG
CCAGTGCCAGACCACCGGCAGGGCCATCAGCCAGAGCGGCGAGCAGACCCTGCTGA
TGATCGACAACACCACCTGCCCCACCGCCGTGCTGGGCAACGTGATCATCAGCCTGG
GCAAGTACCTGGGCAGCGTGAACTACAACAGCGAGGGCATCGCCATCGGCCCCCCC
GTGTTCACCGACAAGGTGGACATCAGCAGCCAGATCAGCAGCATGAACCAGAGCCT
GCAGCAGAGCAAGGACTACATCAAGGAGGCCCAGAGGCTGCTGGACACCGTGAAC
CCCAGCCTG
A345C, E361C, V369C and 1445C mutations (SEQ ID NO: 22):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYCTPMTNNMRECLTGS
TECKCPRELVCSSHVPRFALSNGVLFANCISVTCQCQTTGRAISQSGEQTLLMIDNTTCP
TAVLGNVIISLGKYLGSVNYNSEGIACGPPVFTDKVDISSQISSMNQSLQQSKDYIKEAQR
LLDTVNPSL

SEQ ID NO: 54, below, is an example nucleotide sequence encoding the above amino acid sequence:

ATGGTGGTGATCCTGGACAAGAGGTGCTACTGCAACCTGCTGATCCTGAT
CCTGATGATCAGCGAGTGCAGCGTGGGCATCCTGCACTACGAGAAGCTGAGCAAGA
TCGGCCTGGTGAAGGGCGTGACCAGGAAGTACAAGATCAAGAGCAACCCCCTGACC
AAGGACATCGTGATCAAGATGATCCCCAACGTGAGCAACATGAGCCAGTGCACCGG
CAGCGTGATGGAGAACTACAAGACCAGGCTGAACGGCATCCTGACCCCCATCAAGG
GCGCCCTGGAGATCTACAAGAACAACACCCACGACCTGGTGGGCGACGTGAGGCTG
GCCGGCGTGATCATGGCCGGCGTGGCCATCGGCATCGCCACCGCCGCCCAGATCAC
CGCCGGCGTGGCCCTGTACGAGGCCATGAAGAACGCCGACAACATCAACAAGCTGA
AGAGCAGCATCGAGAGCACCAACGAGGCCGTGGTGAAGCTGCAGGAGACCGCCGA
GAAGACCGTGTACGTGCTGACCGCCCTGCAGGACTACATCAACACCAACCTGGTGC
CCACCATCGACAAGATCAGCTGCAAGCAGACCGAGCTGAGCCTGGACCTGGCCCTG
AGCAAGTACCTGAGCGACCTGCTGTTCGTGTTCGGCCCCAACCTGCAGGACCCCGTG
AGCAACAGCATGACCATCCAGGCCATCAGCCAGGCCTTCGGCGGCAACTACGAGAC
CCTGCTGAGGACCCTGGGCTACGCCACCGAGGACTTCGACGACCTGCTGGAGAGCG
ACAGCATCACCGGCCAGATCATCTACGTGGACCTGAGCAGCTACTACATCATCGTGA
GGGTGTACTTCCCCATCCTGACCGAGATCCAGCAGGCCTACATCCAGGAGCTGCTGC
CCGTGAGCTTCAACAACGACAACAGCGAGTGGATCAGCATCGTGCCCAACTTCATC
CTGGTGAGGAACACCCTGATCAGCAACATCGAGATCGGCTTCTGCCTGATCACCAA
GAGGAGCGTGATCTGCAACCAGGACTACTGCACCCCCATGACCAACAACATGAGGG
AGTGCCTGACCGGCAGCACCTGCAAGTGCCCCAGGGAGCTGGTGTGCAGCAGCCAC
GTGCCCAGGTTCGCCCTGAGCAACGGCGTGCTGTTCGCCAACTGCATCAGCGTGACC
TGCCAGTGCCAGACCACCGGCAGGGCCATCAGCCAGAGCGGCGAGCAGACCCTGCT
GATGATCGACAACACCACCTGCCCCACCGCCGTGCTGGGCAACGTGATCATCAGCCT
GGGCAAGTACCTGGGCAGCGTGAACTACAACAGCGAGGGCATCGCCTGCGGCCCCC
CCGTGTTCACCGACAAGGTGGACATCAGCAGCCAGATCAGCAGCATGAACCAGAGC
CTGCAGCAGAGCAAGGACTACATCAAGGAGGCCCAGAGGCTGCTGGACACCGTGAA
CCCCAGCCTG
Hendra virus (HeV) sequences:
K139P mutation (SEQ ID NO: 23):
MATQEVRLKCLLCGIIVLVLSLEGLGILHYEKLSKIGLVKGITRKYKIKSNPLT
KDIVIKMIPNVSNVSKCTGTVMENYKSRLTGILSPIKGAIELYNNNTHDLVGDVKLAGVV
MAGIAIGIATAAQITAGVALYEAMPNADNINKLKSSIESTNEAVVKLQETAEKTVYVLT
ALQDYINTNLVPTIDQISCKQTELALDLALSKYLSDLLFVFGPNLQDPVSNSMTIQAISQA
FGGNYETLLRTLGYATEDFDDLLESDSIAGQIVYVDLSSYYIIVRVYFPILTEIQQAYVQE
LLPVSFNNDNSEWISIVPNFVLIRNTLISNIEVKYCLITKKSVICNQDYATPMTASVRECLT
GSTDKCPRELVVSSHVPRFALSGGVLFANCISVTCQCQTTGRAISQSGEQTLLMIDNTTC
TTVVLGNIIISLGKYLGSINYNSESIAVGPPVYTDKVDISSQISSMNQSLQQSKDYIKEAQK
ILDTVNPSL

SEQ ID NO: 55, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGCCTAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGACAGCCCTGCAG
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
A345C and V369C mutations (SEQ ID NO: 24):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDLVGDVKLAGVVMAGIAIGIATAAQITAGV
ALYEAMKNADNINKLKSSIESTNEAVVKLQETAEKTVYVLTALQ
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYCTPMTASV
RECLTGSTDKCPRELVCSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 56, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGACAGCCCTGCAG
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACTGCACACCTATGACCGCCAGCGTC
AGAGAGTGTCTGACAGGCAGCACCGACAAGTGCCCCAGAGAACT
GGTGTGCTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
L104C, V114C, L172F and S191P mutations
(SEQ ID NO: 25):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDCVGDVKLAGVCMAGIAIGIATAAQITAGV
ALYEAMKNADNINKLKSSIESTNEAVVKLQETAEKTVYVFTALQ
DYINTNLVPTIDQIPCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 57, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CTGCGTGGGCGACGTGAAACTTGCTGGCGTTTGCATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCAACGAGGCCGTGGTCAAGCTGC
AAGAGACAGCCGAGAAAACCGTGTACGTGTTCACAGCCCTGCAG
GACTACATCAACACCAACCTGGTGCCTACCATCGACCAGATCCC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
A116C and S428C mutations (SEQ ID NO: 26):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDLVGDVKLAGVVMCGIAIGIATAAQITAGV
ALYEAMKNADNINKLKSSIESTNEAVVKLQETAEKTVYVLTALQ
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIICLGKYLGSINYNS
ESIAVGPPVYTDKVDISSQISSMNQSLQQSKDYIKEAQKILDTV
NPSL

SEQ ID NO: 58, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGTGCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGACAGCCCTGCAG
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCTGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
N155P mutation (SEQ ID NO: 27):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDLVGDVKLAGVVMAGIAIGIATAAQITAGV
ALYEAMKNADNINKLKSSIESTPEAVVKLQETAEKTVYVLTALQ
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 59, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCCCCGAGGCCGTGGTCAAGCTGC
AAGAGACAGCCGAGAAAACCGTGTACGTGCTGACAGCCCTGCAG
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
K60I, T173A and Q176I mutations
(SEQ ID NO: 28):
MATQEVRLKCLLCGIIVLVLSLEGLGILHYEKLSKIGLVKGITR
KYKIKSNPLTKDIVIIMIPNVSNVSKCTGTVMENYKSRLTGILS
PIKGAIELYNNNTHDLVGDVKLAGVVMAGIAIGIATAAQITAGV
ALYEAMKNADNINKLKSSIESTNEAVVKLQETAEKTVYVLAALI
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 60, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CATCATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGGCAGCCCTGATC
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
D361C and V445C mutations (SEQ ID NO: 29):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDLVGDVKLAGVVMAGIAIGIATAAQITAGV
ALYEAMKNADNINKLKSSIESTNEAVVKLQETAEKTVYVLTALQ
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTCKCPRELVVSSHVPRFALSGGVLFANCISVTCQCQT
TGRAISQSGEQTLLMIDNTTCTTVVLGNIIISLGKYLGSINYNS
ESIACGPPVYTDKVDISSQISSMNQSLQQSKDYIKEAQKILDTV
NPSL

SEQ ID NO:61, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CAAGATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGACAGCCCTGCAG
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCTGCAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCTGCGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
K60I, K139P, T173A and Q176I mutations
(SEQ ID NO: 30):
MATQEVRLKCLLCGIIVLVLSLEGLGILHYEKLSKIGLVKGITR
KYKIKSNPLTKDIVIIMIPNVSNVSKCTGTVMENYKSRLTGILS
PIKGAIELYNNNTHDLVGDVKLAGVVMAGIAIGIATAAQITAGV
ALYEAMPNADNINKLKSSIESTNEAVVKLQETAEKTVYVLAALI
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 62, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CATCATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGCCTAACGCCGACAACATCAACAAGCT
GAAGTCCAGCATCGAGAGCACCAACGAGGCCGTGGTCAAGCTGC
AAGAGACAGCCGAGAAAACCGTGTACGTGCTGGCAGCCCTGATC
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
K60I, N155P, T173A and Q176I mutations
(SEQ ID NO: 31):
MATQEVRLKCLLCGIIVLVLSLEGLGILHYEKLSKIGLVKGITR
KYKIKSNPLTKDIVIIMIPNVSNVSKCTGTVMENYKSRLTGILS
PIKGAIELYNNNTHDLVGDVKLAGVVMAGIAIGIATAAQITAGV
ALYEAMKNADNINKLKSSIESTPEAVVKLQETAEKTVYVLAALI
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYATPMTASV
RECLTGSTDKCPRELVVSSHVPRFALSGGVLFANCISVTCQCQT
TGRAISQSGEQTLLMIDNTTCTTVVLGNIIISLGKYLGSINYNS
ESIAVGPPVYTDKVDISSQISSMNQSLQQSKDYIKEAQKILDTV
NPSL

SEQ ID NO: 63, below, is an example nucleotide sequence encoding the above amino acid sequence:

ATGGCCACACAAGAAGTGCGGCTGAAGTGCCTGCTGTGCGGCAT
CATTGTGCTGGTGCTGTCTCTGGAAGGCCTGGGCATCCTGCACT
ACGAGAAGCTGTCTAAGATCGGCCTGGTCAAGGGCATCACCCGG
AAGTACAAGATCAAGAGCAACCCTCTGACCAAGGACATCGTGAT
CATCATGATCCCCAACGTGTCCAATGTGTCCAAGTGCACCGGCA
CCGTGATGGAAAACTACAAGAGCCGGCTGACCGGCATTCTGAGC
CCTATCAAGGGCGCCATCGAGCTGTACAACAACAACACCCACGA
CCTCGTGGGCGACGTGAAACTTGCTGGCGTTGTGATGGCCGGAA
TCGCCATCGGAATTGCCACAGCCGCTCAGATTACAGCTGGCGTG
GCCCTGTATGAGGCCATGAAGAACGCCGACAACATCAACAAGCT
GAAGTCCAGCATCGAGAGCACCCCCGAGGCCGTGGTCAAGCTGC
AAGAGACAGCCGAGAAAACCGTGTACGTGCTGGCAGCCCTGATC
GACTACATCAACACCAACCTGGTGCCTACCATCGACCAGATCTC
TTGCAAGCAGACCGAGCTGGCCCTGGATCTGGCCCTGAGCAAGT
ACCTGAGCGACCTGCTGTTCGTGTTCGGCCCCAATCTGCAGGAC
CCTGTGTCCAACAGCATGACCATCCAGGCCATCAGCCAGGCCTT
TGGCGGCAACTATGAGACACTGCTGAGAACCCTGGGCTACGCCA
CCGAGGACTTCGACGATCTGCTGGAAAGCGACTCTATCGCCGGC
CAGATCGTGTATGTGGACCTGAGCAGCTACTACATCATCGTGCG
GGTGTACTTCCCCATCCTGACCGAGATCCAGCAGGCCTACGTGC
AAGAGCTGCTGCCCGTGTCCTTCAACAACGACAACAGCGAGTGG
ATCAGCATCGTGCCCAACTTCGTGCTGATCCGGAACACCCTGAT
CAGCAACATCGAAGTGAAGTACTGCCTGATCACCAAGAAGTCCG
TCATCTGCAACCAGGACTACGCCACACCTATGACCGCCAGCGTC
AGAGAGTGTCTGACAGGCAGCACCGACAAGTGCCCCAGAGAACT
GGTGGTGTCCAGCCACGTGCCAAGATTTGCTCTGTCTGGCGGCG
TGCTGTTCGCCAACTGTATCAGCGTGACCTGCCAGTGCCAGACC
ACAGGCAGAGCCATTTCTCAGAGCGGCGAACAGACCCTGCTGAT
GATCGACAACACCACCTGTACCACCGTGGTGCTGGGCAACATCA
TCATCAGCCTGGGCAAGTATCTGGGCAGCATCAACTACAACTCC
GAGAGCATTGCCGTGGGACCTCCAGTGTACACCGATAAGGTGGA
CATCAGCAGCCAGATCAGCAGCATGAACCAGAGCCTGCAGCAGA
GCAAGGATTACATCAAAGAGGCCCAGAAAATCCTGGACACCGTG
AATCCCAGCCTC
A345C, D361C, V369C and V445C mutations
(SEQ ID NO: 32):
MATQEVRLKCLLCGIIVLVLSLEGLGILHYEKLSKIGLVKGITR
KYKIKSNPLTKDIVIKMIPNVSNVSKCTGTVMENYKSRLTGILS
PIKGAIELYNNNTHDLVGDVKLAGVVMAGIAIGIATAAQITAGV
ALYEAMKNADNINKLKSSIESTNEAVVKLQETAEKTVYVLTALQ
DYINTNLVPTIDQISCKQTELALDLALSKYLSDLLFVFGPNLQD
PVSNSMTIQAISQAFGGNYETLLRTLGYATEDFDDLLESDSIAG
QIVYVDLSSYYIIVRVYFPILTEIQQAYVQELLPVSFNNDNSEW
ISIVPNFVLIRNTLISNIEVKYCLITKKSVICNQDYCTPMTASV
RECLTGSTCKCPRELVCSSHVPRFALSGGVLFANCISVTCQCQT
TGRAISQSGEQTLLMIDNTTCTTVVLGNIIISLGKYLGSINYNS
ESIACGPPVYTDKVDISSQISSMNQSLQQSKDYIKEAQKILDTV
NPSL

SEQ ID NO: 64, below, is an example nucleotide sequence encoding the above amino acid sequence:

ATGGCCACACAAGAAGTGCGGCTGAAGTGCCTGCTGTGCGGCATCATTGT
GCTGGTGCTGTCTCTGGAAGGCCTGGGCATCCTGCACTACGAGAAGCTGT
CTAAGATCGGCCTGGTCAAGGGCATCACCCGGAAGTACAAGATCAAGAGC
AACCCTCTGACCAAGGACATCGTGATCAAGATGATCCCCAACGTGTCCAA
TGTGTCCAAGTGCACCGGCACCGTGATGGAAAACTACAAGAGCCGGCTGA
CCGGCATTCTGAGCCCTATCAAGGGCGCCATCGAGCTGTACAACAACAAC
ACCCACGACCTCGTGGGCGACGTGAAACTTGCTGGCGTTGTGATGGCCGG
AATCGCCATCGGAATTGCCACAGCCGCTCAGATTACAGCTGGCGTGGCCC
TGTATGAGGCCATGAAGAACGCCGACAACATCAACAAGCTGAAGTCCAGC
ATCGAGAGCACCAACGAGGCCGTGGTCAAGCTGCAAGAGACAGCCGAGAA
AACCGTGTACGTGCTGACAGCCCTGCAGGACTACATCAACACCAACCTGG
TGCCTACCATCGACCAGATCTCTTGCAAGCAGACCGAGCTGGCCCTGGAT
CTGGCCCTGAGCAAGTACCTGAGCGACCTGCTGTTCGTGTTCGGCCCCAA
TCTGCAGGACCCTGTGTCCAACAGCATGACCATCCAGGCCATCAGCCAGG
CCTTTGGCGGCAACTATGAGACACTGCTGAGAACCCTGGGCTACGCCACC
GAGGACTTCGACGATCTGCTGGAAAGCGACTCTATCGCCGGCCAGATCGT
GTATGTGGACCTGAGCAGCTACTACATCATCGTGCGGGTGTACTTCCCCA
TCCTGACCGAGATCCAGCAGGCCTACGTGCAAGAGCTGCTGCCCGTGTCC
TTCAACAACGACAACAGCGAGTGGATCAGCATCGTGCCCAACTTCGTGCT
GATCCGGAACACCCTGATCAGCAACATCGAAGTGAAGTACTGCCTGATCA
CCAAGAAGTCCGTCATCTGCAACCAGGACTACTGCACACCTATGACCGCC
AGCGTCAGAGAGTGTCTGACAGGCAGCACCTGCAAGTGCCCCAGAGAACT
GGTGTGCTCCAGCCACGTGCCAAGATTTGCTCTGTCTGGCGGCGTGCTGT
TCGCCAACTGTATCAGCGTGACCTGCCAGTGCCAGACCACAGGCAGAGCC
ATTTCTCAGAGCGGCGAACAGACCCTGCTGATGATCGACAACACCACCTG
TACCACCGTGGTGCTGGGCAACATCATCATCAGCCTGGGCAAGTATCTGG
GCAGCATCAACTACAACTCCGAGAGCATTGCCTGCGGACCTCCAGTGTAC
ACCGATAAGGTGGACATCAGCAGCCAGATCAGCAGCATGAACCAGAGCCT
GCAGCAGAGCAAGGATTACATCAAAGAGGCCCAGAAAATCCTGGACACCG
TGAATCCCAGCCTC

### Example 9

Mutations were made to the LayV F-protein sequence to test for stabilization of the protein in the pre-fusion conformation.

In one example, an E134P amino acid substitution was made. The mutated protein was called LVAM01 (SEQ ID NO: 4). While not wishing to be bound by any theory, a kinked conformation can be introduced to a helix by addition of a proline. A mechanism of fusion activation for LayV-F predicts that the kinked helix that comprises a portion of the fusion peptide and the beginning of HRA can be a high-energy state. This high-energy state can be held in place by the fusion peptide pocket. Disruption of this pocket could allow for straightening of this helix, relieving the tension. By placing a proline near the kink point, this conformation can be stabilized (FIG. 10).

An NiV-F version of the LVAM01 mutant was made (K139P) and was called NVAM01.

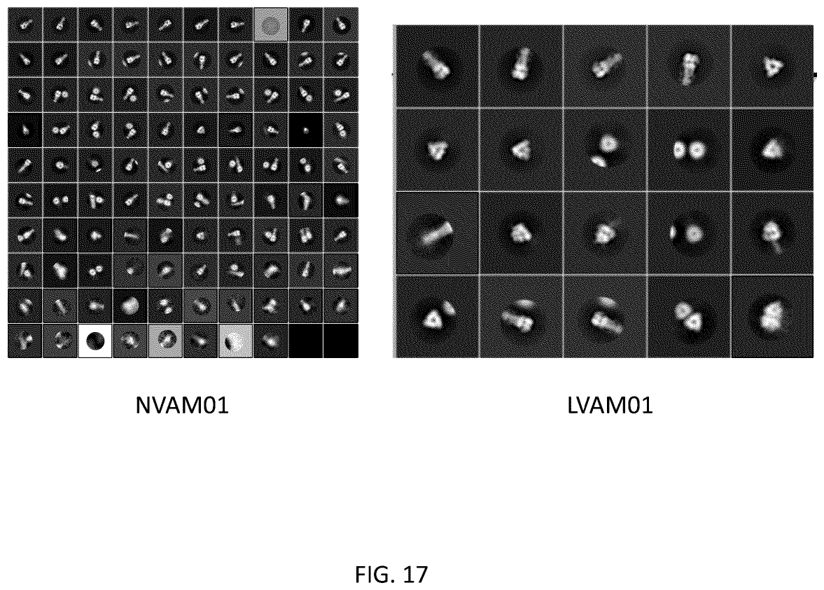

The T data showed that both LVAM01 and NVAM01 were expressed at reasonable levels (750 μg and 180 μg, respectively). In Nipah virus, the conformation was almost exclusively in the post-fusion state (FIG. 17, Left), whereas for the Langya virus version, there was a mix of pre-fusion and post-fusion conformations (FIG. 17, Right). In FIG. 17, the more elongated shapes are characteristic of post-fusion, whereas the more compact, triangular shapes are characteristic of pre-fusion. The ratio of pre-fusion to post-fusion in LVAM01 is similar to that of the wild-type protein (although an exact quantitation is difficult).

That LVAM01 has a mixture of pre-fusion and post-fusion indicates a suitability to iterate on this mutant. We have prepared a further series of proline mutations in a similar area to iterate on this proline stabilization concept (discussed in a later Example). These mutations can change the specific location of the proline or can add an additional proline, to further stabilize in the pre-fusion conformation.

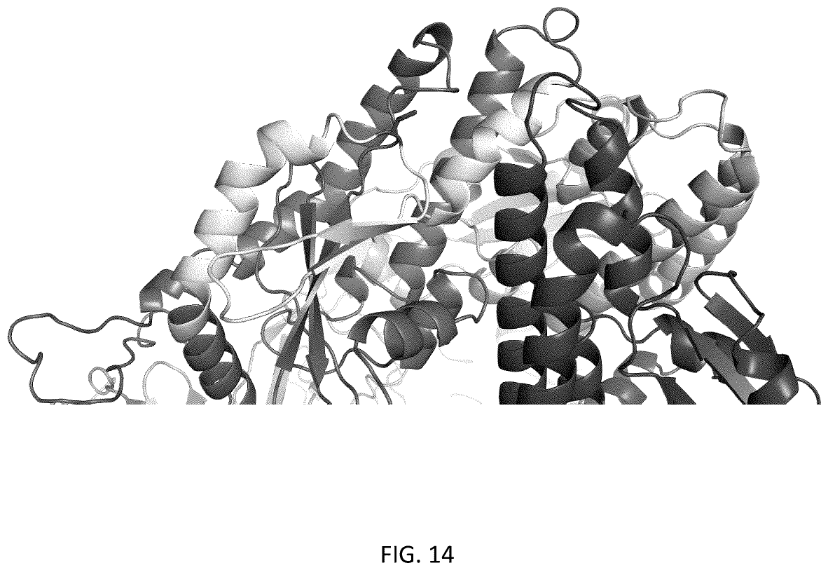

In another example, a Lay V-F was made by addition of an N150P amino acid substitution. The mutated protein was called LVAM05 (SEQ ID NO: 8). While not wishing to be bound by any theory, this linker connects the first part of the HRA helix to the HRA loop in the pre-F conformation. This entire region will straighten into a helix, but a proline at this position may prevent that (FIG. 14). An NiV-F version of the LVAMOS mutant was also made (N155P). For these mutants, the proteins were largely in the post-fusion conformation. In some embodiments, because these mutants are stabilized in the post-fusion conformation, these proteins can be used as reagents for binding or serologic assays. For example, immunization with a stabilized post-fusion form can be used in assays to detect how much of the antibody response in an individual is generated against the post-fusion form of a Henipavirus fusion protein.

### Example 10

Below are amino acid sequences of the further series of proline mutations in the fusion proteins, as mentioned in the previous example. The amino acid sequences are for F proteins in Langya virus or in Nipah virus.

Langya virus (LayV) sequences:
LVAM01.1, N133P mutation (SEQ ID NO: 65):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSPENAQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.2, S132P mutation (SEQ ID NO: 66):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRPNENAQAIANMKNAIQNTNEAVKQLQLANKQTLAVIDT
IRGEINNNIIPVINQLSCDTIGLSVGIKLTQYYSEILTAFGPALQNPVNTRITIQAISSVFNRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
SKCAREKVVSSYVPRFALSDGLVYANCLNTICRCMDTDTPISQSLGTTVSLLDNKKCLV
YQVGDILISVGSYLGEGEYSADNVELGPPVVIDKIDIGNQLAGINQTLQNAEDYIEKSEEF
LKGINPSI
LVAM01.3, R131P mutation (SEQ ID NO: 67):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHPSNENAQAIANMKNAIQNTNEAVKQLQLANKQTLAVIDT
IRGEINNNIIPVINQLSCDTIGLSVGIKLTQYYSEILTAFGPALQNPVNTRITIQAISSVFNRN
FDELLKIMGYTSGDLYEILHSGLIRGNIIDVDVEAGYIALEIEFPNLTLVPNAVVQELMPIS
YNVDGDEWVTLVPRFVLTRTTLLSNIDTSRCTVTESSVICDNDYALPMSYELIGCLQGDT
SKCAREKVVSSYVPRFALSDGLVYANCLNTICRCMDTDTPISQSLGTTVSLLDNKKCLV
YQVGDILISVGSYLGEGEYSADNVELGPPVVIDKIDIGNQLAGINQTLQNAEDYIEKSEEF
LKGINPSI
LVAM01.4, N135P mutation (SEQ ID NO: 68):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNEPAQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.5, A136P mutation (SEQ ID NO: 69):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENPQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.6, Q137P mutation (SEQ ID NO: 70):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAPAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.7, N133P E134P mutations (SEQ ID NO: 71):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSPPNAQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.8, N133P N135P mutations (SEQ ID NO: 72):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSPEPAQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM01.9, E134P N135P mutations (SEQ ID NO: 73):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNPPAQAIANMKNAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM08, N144P mutation (SEQ ID NO: 74):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKPAIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
LVAM09, N144P A145P mutations (SEQ ID NO: 75):
MAFLKSAIICYLLFYPHIVKSSLHYDSLSKVGIIKGLTYNYKIKGSPSTKLMVV
KLIPNIDGVRNCTQKQFDEYKNLVKNVLEPVKLALNAMLDNVKSGNNKYRFAGAIMA
GVALGVATAATVTAGIALHRSNENAQAIANMKPPIQNTNEAVKQLQLANKQTLAVIDTI
RGEINNNIIPVINQLSCDTIGLSVGIKLTQYYSEILTAFGPALQNPVNTRITIQAISSVFNRNF
DELLKIMGYTSGDLYEILHSGLIRGNIIDVDVEAGYIALEIEFPNLTLVPNAVVQELMPISY
NVDGDEWVTLVPRFVLTRTTLLSNIDTSRCTVTESSVICDNDYALPMSYELIGCLQGDTS
KCAREKVVSSYVPRFALSDGLVYANCLNTICRCMDTDTPISQSLGTTVSLLDNKKCLVY
QVGDILISVGSYLGEGEYSADNVELGPPVVIDKIDIGNQLAGINQTLQNAEDYIEKSEEFL
KGINPSI
Nipah virus (NiV) sequences:
NVAM01.1, M138P mutation (SEQ ID NO: 76):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAPKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.2, A137P mutation (SEQ ID NO: 77):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEPMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.3, E136P mutation (SEQ ID NO: 78):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYPAMKNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.4, N140P mutation (SEQ ID NO: 79):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKPADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.5, A141P mutation (SEQ ID NO: 80):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNPDNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.6, D142P mutation (SEQ ID NO: 81):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNAPNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.7, M138P K139P mutations (SEQ ID NO: 82):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAPPNADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.8, M138P N140P mutations (SEQ ID NO: 83):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAPKPADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM01.9, K139P N140P mutations (SEQ ID NO: 84):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMPPADNINKLKSSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM08, S149P mutation (SEQ ID NO: 85):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKPSIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL
NVAM09, S149P S150P mutations (SEQ ID NO: 86):
MVVILDKRCYCNLLILILMISECSVGILHYEKLSKIGLVKGVTRKYKIKSNPLT
KDIVIKMIPNVSNMSQCTGSVMENYKTRLNGILTPIKGALEIYKNNTHDLVGDVRLAGVI
MAGVAIGIATAAQITAGVALYEAMKNADNINKLKPPIESTNEAVVKLQETAEKTVYVLT
ALQDYINTNLVPTIDKISCKQTELSLDLALSKYLSDLLFVFGPNLQDPVSNSMTIQAISQA
FGGNYETLLRTLGYATEDFDDLLESDSITGQIIYVDLSSYYIIVRVYFPILTEIQQAYIQELL
PVSFNNDNSEWISIVPNFILVRNTLISNIEIGFCLITKRSVICNQDYATPMTNNMRECLTGS
TEKCPRELVVSSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQSKDYIKEAQRL
LDTVNPSL

### Example 11

Differential Scanning Fluorimetry (DSF) is a technique that detects conformation conversion of proteins as they unfold during incubation at increasing temperature. Herein is disclosed that DSF can be used to detect the conformation state of proteins, including F proteins, without the need to perform structural studies. In some embodiments, this can be used for Henipavirus F proteins. Disclosed here are studies demonstrating this with Nipah and Langya virus F proteins.

The mechanism for DSF relies on measuring intrinsic fluorescence of proteins at different wavelengths. In some embodiments, 330 and 350 nm are used (e.g., no special dye is used, rather UV light is applied to the protein sample and the fluorescence is measured). The ratio of 330/350 nm signal will typically change depending on the local environment of the fluorescent amino acids (e.g., tryptophan, tyrosine). As the protein is heated, it undergoes conformational changes or denaturation that changes the ratio, resulting in a detectable signal. A typical DSF profile for several proteins is shown in FIG. 18.

Referring to FIG. 18, NiV—F and LayV-F are the wild-type Nipah and Langya fusion proteins. NiVop08 is a stabilized Nipah virus fusion protein that relies primarily on a disulfide bond. The negative peak (i.e., below 0.00 on the x-axis) from ˜45-65° C. for the 3 protein is a conformational conversion peak, which can be indicative of a pre-to-post fusion conversion event. The positive peak from ˜80-90° C. is likely the unfolding of the trimer and final denaturation.

To confirm the above, the proteins were pre-incubated at 60° C. for 2 hours. The same DSF analysis was then performed and confirmed by scanning electron microscopy. FIG. 19 shows the DSF results. Shown is that, after heat incubation, both wild-type proteins had their negative peaks eliminated (dashed lines). No peaks were seen for these proteins during the, 45-60° C. part of the heat ramp experiment, indicating no changes in conformation of these proteins (since they had been heated to 60° C. already, there was no pre- to post-fusion conversion). There was no change in the tracing for the NiVop08 protein (e.g., for the negative peak between about 60-70° C.) compared to FIG. 18).

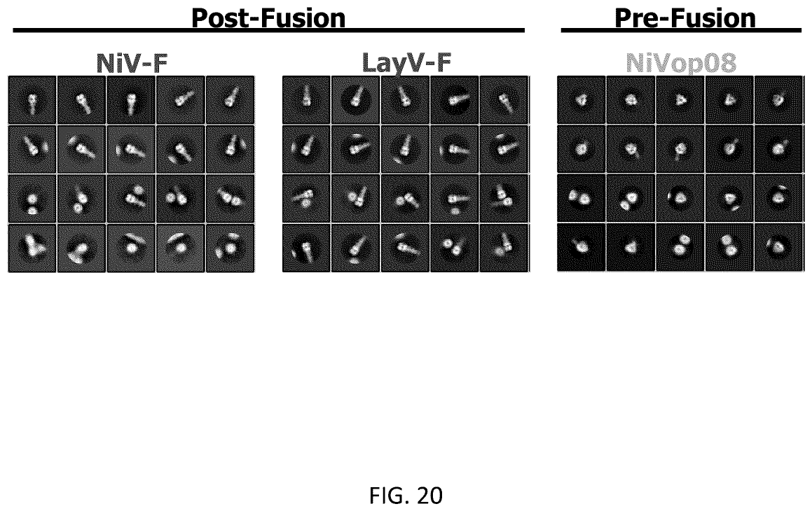

The data shown in FIG. 20 confirmed these conclusions. These data indicate that NiV-F and LayV-F have the characteristic post-fusion shape, whereas NiVop08 retains the pre-fusion shape.

This new application of DSF is to specifically look for the presence or absence of the initial negative peak from ˜45-50° C. as an indication of conformational state. In some embodiments, a DSF peak (e.g., a “negative” peak), that occurs between ˜45-65° C., that is eliminated when DSF is performed on a wt F-protein already preheated to 60° C., is indicative of pre- to post-fusion transition. When the above condition is met, screens for mutants in which the wt DSF peak is not eliminated with a temperature increase can indicate a mutant that is stabilized in the pre-fusion conformation.

In some embodiments, DSF can show persistence of the negative peak after pretreatment. In some embodiments, DSF can show a right-shifting of a peak as compared to wild-type and can indicate that the energy barrier to conversion has been raised.

### Example 12

Detection of conformation conversion through antibodies is shown.

Bio-Layer Interferometry (BLI) is an assay used to detect binding of a protein in solution to an immobilized component on a tip inserted into solution. Here, we immobilized antibodies that bind to Nipah virus. Two antibodies were used in these studies. The first antibody, 1H1, binds to both the pre- and post-fusion conformations of NiV-F. The second antibody, 4B8, only binds to the pre-fusion conformation of NiV-F. In these studies, heat treatment conditions were 50° C. for 15 minutes.

In FIG. 21, HT means “heat treated.” The data show that NiV-F, which can convert from pre-fusion to post-fusion conformation, can bind to the H1 antibody both before and after heating. The data show that NiV-F can bind to the 4B8 antibody when the NiV-F has not been heated. When NiV-F has been heated (and has transitioned to the post-fusion conformation), it does not bind to antibody 4B8.

In FIG. 22, NiVop08, which is unaffected by heat treatment, still binds to both antibodies after heat treatment, indicating its conformational state has not changed.

### Example 13

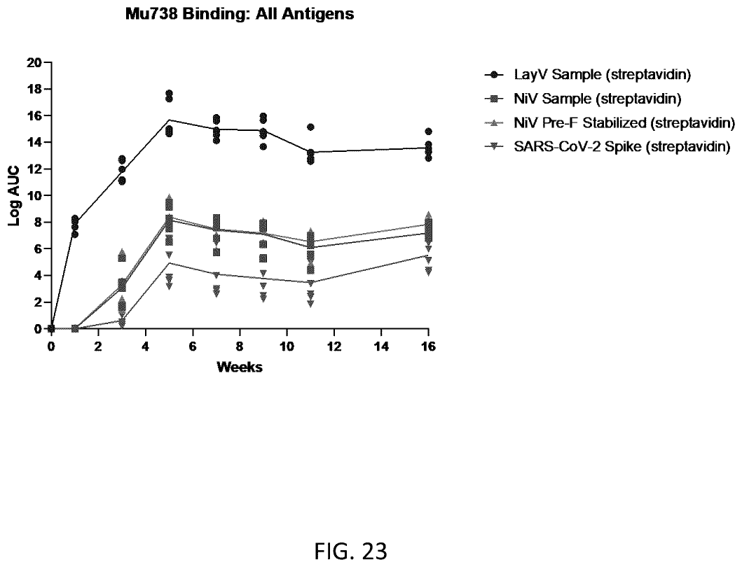

FIG. 23 shows results from wild-type Langya virus immunization of mice. The data show binding of sera from Langya virus-immunized Mu737 mice to LayV NiV and pre-fusion stabilized NiV. Negative control binding to SARS-COV-2 spike is also shown.

The data show that antibodies in the sera bound to LayV, and lesser so to NiV and pre-fusion stabilized NiV. These data show cross-reactivity of antibodies stimulated by LayV to NiV.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. Brown N P, Leroy C, Sander C. 1998. MView: a web-compatible database search or multiple alignment viewer. Bioinformatics 14:380-1.
2. Xu K, Chan Y-P, Bradel-Tretheway B, Akyol-Ataman Z, Zhu Y, Dutta S, Yan L, Feng Y, Wang L-F, Skiniotis G, Lee B, Zhou Z H, Broder C C, Aguilar H C, Nikolov D B. 2015. Crystal Structure of the Pre-fusion Nipah Virus Fusion Glycoprotein Reveals a Novel Hexamer-of-Trimers Assembly. PLOS Pathogens 11: e1005322.
3. Wong J J W, Paterson R G, Lamb R A, Jardetzky T S. 2016. Structure and stabilization of the Hendra virus F glycoprotein in its prefusion form. Proceedings of the National Academy of Sciences 113:1056-1061.
4. Stewart-Jones G B E, Chuang G-Y, Xu K, Zhou T, Acharya P, Tsybovsky Y, Ou L, Zhang B, Fernandez-Rodriguez B, Gilardi V, Silacci-Fregni C, Beltramello M, Baxa U, Druz A, Kong W-P, Thomas P V, Yang Y, Foulds K E, Todd J-P, Wei H, Salazar A M, Scorpio D G, Carragher B, Potter C S, Corti D, Mascola J R, Lanzavecchia A, Kwong P D. 2018. Structure-based design of a quadrivalent fusion glycoprotein vaccine for human parainfluenza virus types 1-4. Proceedings of the National Academy of Sciences 115:12265-12270.
5. Avanzato V A, Oguntuyo K Y, Escalera-Zamudio M, Gutierrez B, Golden M, Kosakovsky Pond S L, Pryce R, Walter T S, Seow J, Doores K J, Pybus O G, Munster V J, Lee B, Bowden T A. 2019. A structural basis for antibody-mediated neutralization of Nipah virus reveals a site of vulnerability at the fusion glycoprotein apex. Proceedings of the National Academy of Sciences 116:25057-25067.
6. Dang H V, Cross R W, Borisevich V, Bornholdt Z A, West B R, Chan Y-P, Mire C E, Da Silva S C, Dimitrov A S, Yan L, Amaya M, Navaratnarajah C K, Zeitlin L, Geisbert T W, Broder C C, Veesler D. 2021. Broadly neutralizing antibody cocktails targeting Nipah virus and Hendra virus fusion glycoproteins. Nature Structural & amp; Molecular Biology 28:426-434.
7. Pei J, Grishin N V. 2001. AL2CO: calculation of positional conservation in a protein sequence alignment. Bioinformatics 17:700-12.
8. Welch B D, Liu Y, Kors C A, Leser G P, Jardetzky T S, Lamb R A. 2012. Structure of the cleavage-activated prefusion form of the parainfluenza virus 5 fusion protein. Proceedings of the National Academy of Sciences 109:16672-16677.
9. Liu Q, Stone J A, Bradel-Tretheway B, Dabundo J, Benavides Montano J A, Santos-Montanez J, Biering S B, Nicola A V, Iorio R M, Lu X, Aguilar H C. 2013. Unraveling a Three-Step Spatiotemporal Mechanism of Triggering of Receptor-Induced Nipah Virus Fusion and Cell Entry. PLOS Pathogens 9: e1003770.
10. Loomis R J, Stewart-Jones G B E, Tsybovsky Y, Caringal R T, Morabito K M, Mclellan J S, Chamberlain A L, Nugent S T, Hutchinson G B, Kueltzo L A, Mascola J R, Graham B S. 2020. Structure-Based Design of Nipah Virus Vaccines: A Generalizable Approach to Paramyxovirus Immunogen Development. Frontiers in Immunology 11.
11. Dang H V, Chan Y-P, Park Y-J, Snijder J, Da Silva S C, Vu B, Yan L, Feng Y-R, Rockx B, Geisbert T W, Mire C E, Broder C C, Veesler D. 2019. An antibody against the F glycoprotein inhibits Nipah and Hendra virus infections. Nature Structural & amp; Molecular Biology 26:980-987.
12. Punjani A, Rubinstein J L, Fleet D J, Brubaker M A. 2017. cryoSPARC: algorithms for rapid unsupervised cryo-EM structure determination. Nature Methods 14:290-296.
13. Punjani A, Zhang H, Fleet D J. 2020. Non-uniform refinement: adaptive regularization improves single-particle cryo-EM reconstruction. Nature Methods 17:1214-1221.
14. Lee J E, Fusco M L, Hessell A J, Oswald W B, Burton D R, Saphire E O. 2008. Structure of the Ebola virus glycoprotein bound to an antibody from a human survivor. Nature 454:177-182.
15. Cai Y, Zhang J, Xiao T, Peng H, Sterling S M, Walsh R M, Rawson S, Rits-Volloch S, Chen B. 2020. Distinct conformational states of SARS-COV-2 spike protein. Science 369:1586-1592.
16. Benton D J, Nans A, Calder L J, Turner J, Neu U, Lin Y P, Ketelaars E, Kallewaard N L, Corti D, Lanzavecchia A, Gamblin S J, Rosenthal P B, Skehel J J. 2018. Influenza hemagglutinin membrane anchor. Proceedings of the National Academy of Sciences 115:10112-10117.
17. Ozorowski G, Pallesen J, de Val N, Lyumkis D, Cottrell C A, Torres J L, Copps J, Stanfield R L, Cupo A, Pugach P, Moore J P, Wilson I A, Ward A B. 2017. Open and closed structures reveal allostery and pliability in the HIV-1 envelope spike. Nature 547:360-363.
18. Kong R, Xu K, Zhou T, Acharya P, Lemmin T, Liu K, Ozorowski G, Soto C, Taft J D, Bailer R T, Cale E M, Chen L, Choi C W, Chuang G-Y, Doria-Rose N A, Druz A, Georgiev I S, Gorman J, Huang J, Joyce M G, Louder M K, Ma X, McKee K, O'Dell S, Pancera M, Yang Y, Blanchard S C, Mothes W, Burton D R, Koff W C, Connors M, Ward A B, Kwong P D, Mascola J R. 2016. Fusion peptide of HIV-1 as a site of vulnerability to neutralizing antibody. Science 352:828-833.
19. Pancera M, Zhou T, Druz A, Georgiev I S, Soto C, Gorman J, Huang J, Acharya P, Chuang G-Y, Ofek G, Stewart-Jones G B E, Stuckey J, Bailer R T, Joyce M G, Louder M K, Tumba N, Yang Y, Zhang B, Cohen M S, Haynes B F, Mascola J R, Morris L, Munro J B, Blanchard S C, Mothes W, Connors M, Kwong P D. 2014. Structure and immune recognition of trimeric pre-fusion HIV-1 Env. Nature 514:455-461.
20. Shi W, Cai Y, Zhu H, Peng H, Voyer J, Rits-Volloch S, Cao H, Mayer M L, Song K, Xu C, Lu J, Zhang J, Chen B. 2022. Cryo-EM structure of SARS-COV-2 postfusion spike in membrane. bioRxiv doi: 10.1101/2022.12.05.519151:2022.12.05.519151.
21. Zhang X-A, Li H, Jiang F-C, Zhu F, Zhang Y-F, Chen J-J, Tan C-W, Anderson D E, Fan H, Dong L-Y, Li C, Zhang P-H, Li Y, Ding H, Fang L-Q, Wang L-F, Liu W. 2022. A Zoonotic Henipavirus in Febrile Patients in China. New England Journal of Medicine 387:470-472.
22. Wu Z, Yang L, Yang F, Ren X, Jiang J, Dong J, Sun L, Zhu Y, Zhou H, Jin Q. 2014. Novel Henipa-like virus, Mojiang Paramyxovirus, in rats, China, 2012. Emerg Infect Dis 20:1064-6.
23. Lee S-H, Kim K, Kim J, No J S, Park K, Budhathoki S, Lee S H, Lee J, Cho S H, Cho S, Lee G-Y, Hwang J, Kim H-C, Klein T A, Uhm C-S, Kim W-K, Song J-W. 2021. Discovery and Genetic Characterization of Novel Paramyxoviruses Related to the Genus Henipavirus in Crocidura Species in the Republic of Korea. Viruses 13:2020.
24. Field H, Young P, Yob J M, Mills J, Hall L, Mackenzie J. 2001. The natural history of Hendra and Nipah viruses. Microbes Infect 3:307-14.
25. Rima B, Balkema-Buschmann A, Dundon W G, Duprex P, Easton A, Fouchier R, Kurath G, Lamb R, Lee B, Rota P, Wang L, Ictv Report C. 2019. ICTV Virus Taxonomy Profile: Paramyxoviridae. J Gen Virol 100:1593-1594.
26. Anonymous. Prioritizing diseases for research and development in emergency contexts, on WHO. https://www.who.int/activities/prioritizing-diseases-for-research-and-development-in-emergency-contexts. Accessed Feb. 28, 2023.
27. Aguilar H C, Iorio R M. 2012. Henipavirus Membrane Fusion and Viral Entry, p 79-94. In Lee B, Rota P A (ed), Henipavirus: Ecology, Molecular Virology, and Pathogenesis doi: 10.1007/82_2012_200. Springer Berlin Heidelberg, Berlin, Heidelberg.
28. Negrete O A, Levroney E L, Aguilar H C, Bertolotti-Ciarlet A, Nazarian R, Tajyar S, Lee B. 2005. EphrinB2 is the entry receptor for Nipah virus, an emergent deadly paramyxovirus. Nature 436:401-5.
29. Xu K, Rajashankar K R, Chan Y-P, Himanen J P, Broder C C, Nikolov D B. 2008. Host cell recognition by the henipaviruses: Crystal structures of the Nipah G attachment glycoprotein and its complex with ephrin-B3. Proceedings of the National Academy of Sciences 105:9953-9958.
30. White J M, Delos S E, Brecher M, Schornberg K. 2008. Structures and mechanisms of viral membrane fusion proteins: multiple variations on a common theme. Crit Rev Biochem Mol Biol 43:189-219.
31. Lamb R A, Jardetzky T S. 2007. Structural basis of viral invasion: lessons from paramyxovirus F. Current Opinion in Structural Biology 17:427-436.
32. Pager C T, Dutch R E. 2005. Cathepsin L is involved in proteolytic processing of the Hendra virus fusion protein. J Virol 79:12714-20.
33. Bose S, Jardetzky T S, Lamb R A. 2015. Timing is everything: Fine-tuned molecular machines orchestrate paramyxovirus entry. Virology 479-480:518-31.
34. Iorio R M, Melanson V R, Mahon P J. 2009. Glycoprotein interactions in paramyxovirus fusion. Future Virol 4:335-351.
35. Hsieh C-L, Goldsmith J A, Schaub J M, DiVenere A M, Kuo H-C, Javanmardi K, Le K C, Wrapp D, Lee A G, Liu Y, Chou C-W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, Mclellan J S. 2020. Structure-based design of prefusion-stabilized SARS-COV-2 spikes. Science 369:1501-1505.
36. Wrapp D, Wang N, Corbett K S, Goldsmith J A, Hsieh C-L, Abiona O, Graham B S, Mclellan J S. 2020. Cryo-EM structure of the 2019-nCOV spike in the prefusion conformation. Science 367:1260-1263.
37. Do Kwon Y, Pancera M, Acharya P, Georgiev I S, Crooks E T, Gorman J, Joyce M G, Guttman M, Ma X, Narpala S, Soto C, Terry D S, Yang Y, Zhou T, Ahlsen G, Bailer R T, Chambers M, Chuang G-Y, Doria-Rose N A, Druz A, Hallen M A, Harned A, Kirys T, Louder M K, O'Dell S, Ofek G, Osawa K, Prabhakaran M, Sastry M, Stewart-Jones G B E, Stuckey J, Thomas P V, Tittley T, Williams C, Zhang B, Zhao H, Zhou Z, Donald B R, Lee L K, Zolla-Pazner S, Baxa U, Schön A, Freire E, Shapiro L, Lee K K, Arthos J, Munro J B, Blanchard S C, Mothes W, Binley J M, et al. 2015. Crystal structure, conformational fixation and entry-related interactions of mature ligand-free HIV-1 Env. Nature Structural & Molecular Biology 22:522-531.
38. Rawi R, Rutten L, Lai Y-T, Olia A S, Blokland S, Juraszek J, Shen C-H, Tsybovsky Y, Verardi R, Yang Y, Zhang B, Zhou T, Chuang G-Y, Kwong P D, Langedijk J P M. 2020. Automated Design by Structure-Based Stabilization and Consensus Repair to Achieve Prefusion-Closed Envelope Trimers in a Wide Variety of HIV Strains. Cell Reports 33:108432.
39. Tan Y Z, Baldwin P R, Davis J H, Williamson J R, Potter C S, Carragher B, Lyumkis D. 2017. Addressing preferred specimen orientation in single-particle cryo-EM through tilting. Nature Methods 14:793-796.
40. Hashiguchi T, Fukuda Y, Matsuoka R, Kuroda D, Kubota M, Shirogane Y, Watanabe S, Tsumoto K, Kohda D, Plemper R K, Yanagi Y. 2018. Structures of the prefusion form of measles virus fusion protein in complex with inhibitors. Proceedings of the National Academy of Sciences 115:2496-2501.
41. Yin H S, Wen X, Paterson R G, Lamb R A, Jardetzky T S. 2006. Structure of the parainfluenza virus 5 F protein in its metastable, prefusion conformation. Nature 439:38-44.
42. Murray K, Selleck P, Hooper P, Hyatt A, Gould A, Gleeson L, Westbury H, Hiley L, Selvey L, Rodwell B, Ketterer P. 1995. A Morbillivirus that Caused Fatal Fisease in Horses and Humans. Science 268:94-97.
43. Madera S, Kistler A, Ranaivoson H C, Ahyong V, Andrianiaina A, Andry S, Raharinosy V, Randriambolamanantsoa T H, Ravelomanantsoa N A F, Tato C M, DeRisi J L, Aguilar H C, Lacoste V, Dussart P, Heraud J-M, Brook C E. 2022. Discovery and Genomic Characterization of a Novel Henipavirus, Angavokely Virus, from Fruit Bats in Madagascar. Journal of Virology 96: e00921-22.
44. Hernández LHA, da Paz T Y B, Silva S Pd, Silva F Sd, Barros B CVd, Nunes B T D, Casseb L M N, Medeiros D B A, Vasconcelos P FdC, Cruz A C R. 2022. First Genomic Evidence of a Henipa-like Virus in Brazil. Viruses 14:2167.
45. Aguilar H C, Matreyek K A, Filone C M, Hashimi S T, Levroney E L, Negrete O A, Bertolotti-Ciarlet A, Choi D Y, McHardy I, Fulcher J A, Su S V, Wolf M C, Kohatsu L, Baum L G, Lee B. 2006. N-glycans on Nipah virus fusion protein protect against neutralization but reduce membrane fusion and viral entry. J Virol 80:4878-89.
46. Carter J R, Pager C T, Fowler S D, Dutch R E. 2005. Role of N-linked glycosylation of the Hendra virus fusion protein. J Virol 79:7922-5.
47. Yin H-S, Paterson R G, Wen X, Lamb R A, Jardetzky T S. 2005. Structure of the uncleaved ectodomain of the paramyxovirus (hPIV3) fusion protein. Proceedings of the National Academy of Sciences 102:9288-9293.
48. Yeo Y Y, Buchholz D W, Gamble A, Jager M, Aguilar H C. 2021. Headless Henipaviral Receptor Binding Glycoproteins Reveal Fusion Modulation by the Head/Stalk Interface and Post-receptor Binding Contributions of the Head Domain. Journal of Virology 95: e00666-21.
49. Bose S, Heath C M, Shah P A, Alayyoubi M, Jardetzky T S, Lamb R A. 2013. Mutations in the parainfluenza virus 5 fusion protein reveal domains important for fusion triggering and metastability. J Virol 87:13520-31.
50. Poor T A, Jones L M, Sood A, Leser G P, Plasencia M D, Rempel D L, Jardetzky T S, Woods R J, Gross M L, Lamb R A. 2014. Probing the paramyxovirus fusion (F) protein-refolding event from pre- to postfusion by oxidative footprinting. Proceedings of the National Academy of Sciences 111: E2596-E2605.
51. Wilman H R, Shi J, Deane C M. 2014. Helix kinks are equally prevalent in soluble and membrane proteins. Proteins 82:1960-70.
52. McCarthy K R, Lee J, Watanabe A, Kuraoka M, Robinson-Mccarthy L R, Georgiou G, Kelsoe G, Harrison S C. 2021. A Prevalent Focused Human Antibody Response to the Influenza Virus Hemagglutinin Head Interface. mBio 12: e01144-21.
53. Grant T, Grigorieff N. 2015. Measuring the optimal exposure for single particle cryo-EM using a 2.6 Å reconstruction of rotavirus VP6. Elife 4: e06980.
54. Liebschner D, Afonine P V, Baker M L, Bunkoczi G, Chen V B, Croll T I, Hintze B, Hung L-W, Jain S, McCoy A J, Moriarty N W, Oeffner R D, Poon B K, Prisant M G, Read R J, Richardson J S, Richardson D C, Sammito M D, Sobolev O V, Stockwell D H, Terwilliger T C, Urzhumtsev A G, Videau L L, Williams C J, Adams P D. 2019. Macromolecular structure determination using X-rays, neutrons and electrons: recent developments in Phenix. Acta Crystallographica Section D 75:861-877.
55. Anonymous. The PyMOL Molecular Graphics System, v2.0. Schrödinger, LLC,
56. Pettersen E F, Goddard T D, Huang C C, Meng E C, Couch G S, Croll T I, Morris J H, Ferrin T E. 2021. UCSF ChimeraX: Structure visualization for researchers, educators, and developers. Protein Sci 30:70-82.
57. Croll T. 2018. ISOLDE: a physically realistic environment for model building into low-resolution electron-density maps. Acta Crystallographica Section D 74:519-530.
58. Madeira F, Pearce M, Tivey A R N, Basutkar P, Lee J, Edbali O, Madhusoodanan N, Kolesnikov A, Lopez R. 2022. Search and sequence analysis tools services from EMBL-EBI in 2022. Nucleic acids research doi: 10.1093/nar/gkac240: gkac240.

1. A Henipavirus fusion protein antigen having an amino acid sequence at least 80% identical to SEQ ID NO: 1-3 and wherein the antigen sequence comprises one or more of the following mutations, or a mutation at a homologous position thereof:
a) glutamic acid 134 to proline;
b) alanine 340 to cysteine and/or valine 364 to cysteine;
c) glycine 99 to cysteine, isoleucine 109 to cysteine, isoleucine 167 to phenylalanine and serine 186 to proline;
d) alanine 111 to cysteine and serine 423 to cysteine;
e) asparagine 150 to proline;
f) lysine 55 to isoleucine, aspartic acid 168 to alanine, and arginine 171 to isoleucine;
g) serine 356 to cysteine and lysine 440 to cysteine;
h) a combination of the mutations of a) and f); and
i) the combination of mutations of b) and g).

2. The Henipavirus fusion protein antigen of claim 1, having an amino acid sequence at least 80% identical to SEQ ID NO: 1 and having an amino acid substitution at A11, K55, G99, I109, E134, N150, I167, D168, R171, S186, A340, S356, V364, S365, S424, L440, or combinations thereof.

3. The Henipavirus fusion protein antigen of claim 2, having an amino acid substitution A11C, K55I, G99C, I109C, E134P, N150P, I167F, D168A, R171I, S186P, A340C, S356C, V364C, S365C, S424C, L440C, or combinations thereof.

4. The Henipavirus fusion protein antigen of claim 1, having an amino acid sequence at least 80% identical to SEQ ID NO: 1.

5. The Henipavirus fusion protein antigen of claim 1, having an amino acid sequence at least 80% identical to SEQ ID NO: 2 and having an amino acid substitution at K60, A116, K139, N155, T173, Q176, A345, E361, V369, S428, I445, or combinations thereof.

6. The Henipavirus fusion protein antigen of claim 5, having an amino acid substitution K60I, A116C, K139P, N155P, T173A, Q176I, A345C, E361I or E361C, V369C, S428C, I445C, or combinations thereof.

7. The Henipavirus fusion protein antigen of claim 1, having an amino acid sequence at least 80% identical to SEQ ID NO: 3 and having an amino acid substitution at K60, L104, V114, A116, K139, N155, L172, T173, Q176, S191, A345, D361, V369, S428, V445, or combinations thereof.

8. The Henipavirus fusion protein antigen of claim 1, comprising an amino acid sequence at least 80% identical to any one of SEQ ID NOs: 4-32.

9. An immunogenic composition, comprising the Henipavirus fusion protein antigen of claim 1, and a pharmaceutically acceptable carrier.

10. A method for generating an immune response in a subject, comprising administering to the subject an effective amount of the immunogenic composition of claim 9.

11. A nucleotide sequence encoding the Henipavirus fusion protein antigen of claim 1.

12. A vector encoding the nucleotide sequence of claim 11.

13. A cell comprising the nucleotide sequence of claim 11.

14. A Henipavirus fusion protein antigen at least 80% identical to SEQ ID NO: 1, having N133P (SEQ ID NO: 65), S132P (SEQ ID NO: 66), R131P (SEQ ID NO: 67), N135P (SEQ ID NO: 68), A136P (SEQ ID NO: 69), Q137P (SEQ ID NO: 70), N133P and E134P (SEQ ID NO: 71), N133P and N135P (SEQ ID NO: 72), E134P and N135P (SEQ ID NO: 73), N144P (SEQ ID NO: 74), or N144P and A145P (SEQ ID NO: 75).

15. A Henipavirus fusion protein antigen at least 80% identical to SEQ ID NO: 2, having M138P (SEQ ID NO: 76), A137P (SEQ ID NO: 77), E136P (SEQ ID NO: 78), N140P (SEQ ID NO: 79, A141P (SEQ ID NO: 80), D142P (SEQ ID NO: 81), M138P and K139P (SEQ ID NO: 82), M138P and N140P (SEQ ID NO: 83), K139P and N140P (SEQ ID NO: 84), S149P (SEQ ID NO: 85), and S149P and S150P (SEQ ID NO: 86).

16. A Henipavirus fusion protein having an amino acid sequence at least 80% identical to SEQ ID NO: 1-3, wherein the fusion protein is stabilized in a post-fusion conformation.

17. The Henipavirus fusion protein of claim 16, from Langya virus and comprising N150P (SEQ ID NO: 8) or an amino acid sequence at least 80% identical thereto.

18. The Henipavirus fusion protein of claim 16, from Nipah virus and comprising N155P or an amino acid sequence at least 80% identical thereto.

19. A method for screening a viral fusion protein for capability to transition from a pre-fusion to a post-fusion conformation, comprising:
determining intrinsic fluorescence or autofluorescence of the viral fusion protein at different wavelengths while the viral fusion protein is heated (differential scanning fluorimetry or DSF); and
comparing a DSF tracing profile of the protein obtained from the fluorescence determining, with a DSF tracing profile of a control protein.

20. A method for screening a viral fusion protein for capability to transition from a pre-fusion to a post-fusion conformation, comprising:
contacting a heated viral fusion protein and an unheated viral fusion protein with an antibody that differentially binds to the viral fusion protein dependent on a pre-fusion or post-fusion conformation of the protein; and
determining binding of the antibody to the heated viral fusion protein and to the unheated viral fusion protein.
